(19) United States
(12) Patent Application Publication
(10) Pub. No.: US 2026/0092062 A1
(43) Pub. Date: Apr. 2, 2026

(54) PYRIDINE CARBOXAMIDE COMPOUNDS AND THEIR USE IN TREATING MEDICAL CONDITIONS

(71) Applicant: Rectify Pharmaceuticals, Inc., Boston, MA (US)

(72) Inventors: Todd Bosanac, Burlington, CT (US); Nathan Oliver Fuller, Arlington, MA (US); Robert Owen Hughes, Newtown, CT (US); Guy Bemis, San Diego, CA (US); Janeta Valentina Popovici Muller, Windham, NH (US)

(21) Appl. No.: 19/113,556

(22) PCT Filed: Sep. 26, 2023
(86) PCT No.: PCT/US2023/033708

§ 371 Date: Mar. 20, 2025

Related U.S. Application Data
(60) Provisional application No. 63/410,005, filed on Sep. 26, 2022.

(51) Int. Cl. C07D 471/04; A61K 31/444; A61K 31/4545; A61K 31/4725; A61K 31/506; A61K 31/519; A61K 31/5377; A61K 31/538; C07D 519/00

(52) U.S. Cl. CPC C07D 471/04; A61K 31/444; A61K 31/4545; A61K 31/4725; A61K 31/506; A61K 31/519; A61K 31/5377; A61K 31/538; C07D 519/00

(57) ABSTRACT

The invention provides pyridine carboxamide compounds, pharmaceutical compositions, and their use in treating medical conditions.

### CROSS-REFERENCE TO RELATED APPLICATIONS

This application claims the benefit of and priority to U.S. Provisional Patent Application Ser. No. 63/410,005, filed Sep. 26, 2022, the contents of which are hereby incorporated by reference in their entirety.

### FIELD OF THE INVENTION

The invention provides pyridine carboxamide compounds, pharmaceutical compositions, and their use in treating medical conditions.

### BACKGROUND

ATP-binding cassette (ABC) transporters are a large, phylogenetically conserved gene family with broad physiological and pathological relevance.[1,2] They are expressed throughout the body and transport a diverse range of substrates across lipid membranes. ABC transporters are transmembrane. ATP-binding proteins that use the energy released during ATP hydrolysis to move substrates from one side of a lipid membrane to the other.[2.3]

At least 21 ABC transporters underly rare monogenic disorders with even more implicated in the predisposition to and symptomology of common and complex diseases. Such broad (patho)physiological relevance places this class of proteins at the intersection of disease causation and therapeutic potential, underlining them as promising targets for drug discovery. Based on a handful of characterized disorders, including cystic fibrosis (CF), progressive familial intrahepatic cholestasis 2 (PFIC2), and Stargardt disease (STGD), there is a mechanistic commonality to pathogenic ABC transporter missense mutations, principally their impact on protein folding leading to endoplasmic reticulum (ER) degradation (trafficking defects), or protein function, leading to decreased substrate transport (transport defects). Importantly, small molecule compounds may enable treatment of disease symptoms not directly caused by mutations in ABC transporter genes.

There is a need for small molecule compounds that can positively modulate ABC transporter function and therefore be used for the treatment of diseases in which such enhancement is predicted to be of therapeutic value, either in modifying disease or in treating disease symptomology. There is a need for modulators that can address mutations in ABCB11, ABCB4, ABCC6. ABCA4, ABCD1, ABCA1 and ABCA7 for the treatment of PFIC2, PFIC3, PXE/GACI, STGD, X-ALD and Alzheimer's disease, respectively.

Accordingly, the need exists for new therapeutic methods and compounds for treating ABC transporter dysfunction. The present invention addresses the foregoing needs and provides other related advantages.

### SUMMARY

The invention provides pyridine carboxamide compounds, pharmaceutical compositions, and their use in treating disorders associated with ABC transporter dysfunction. In particular, one aspect of the invention provides a collection of pyridine carboxamide compounds, such as a compound represented by Formula I:

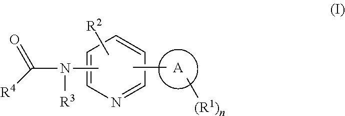

or a pharmaceutically acceptable salt thereof, where the variables are as defined in the detailed description. Further description of additional collections of pyridine carboxamide compounds are described in the detailed description. The compounds may be part of a pharmaceutical composition comprising a pharmaceutically acceptable carrier.

Another aspect of the invention provides a method of treating disorders associated with ABC transporter dysfunction. The method comprises administering to a subject in need thereof a therapeutically effective amount of a compound described herein, such as a compound of Formula I, to treat the disorder associated with ABC transporter dysfunction, as further described in the detailed description.

### DETAILED DESCRIPTION

In certain embodiments, the present disclosure provides a compound of Formula I:

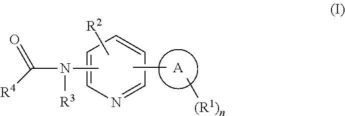

or a pharmaceutically acceptable salt thereof, wherein
Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur, a 4-8 membered saturated or partially unsaturated monocyclic heterocyclic ring containing 1-4 heteroatoms independently selected from nitrogen, oxygen, and sulfur, a 5-6 membered monocyclic heteroaromatic ring having 1-4 heteroatoms independently selected from nitrogen, oxygen, and sulfur, a 6-11 membered saturated or partially unsaturated bicyclic carbocyclic ring, a 3-8 membered saturated or partially unsaturated monocyclic carbocyclic ring, an 8-10 membered bicyclic aromatic carbocyclic ring, or phenyl;
R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, C₁₋₄ haloaliphatic, or a cyclic group selected from a 3-8 membered saturated monocyclic carbocyclic ring or a 3-8 membered saturated monocyclic heterocyclic ring having 1-4 heteroatoms independently selected from nitrogen, oxygen, and sulfur;
R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy;
R³ is hydrogen, cyclopropyl, or C₁₋₃ alkyl optionally substituted with a group selected from —OH, OMe, —NH₂, —NH(CH₃), and —N(CH₃)₂;
R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵;
R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur;
R represents independently for each occurrence hydrogen, hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring;
n is 0, 1, 2, or 3; and
p is 0, 1, 2, 3 or 4.

The invention further provides compounds of Formula I and pharmaceutical compositions and their use in treating disorders associated with ABC transporter dysfunction.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

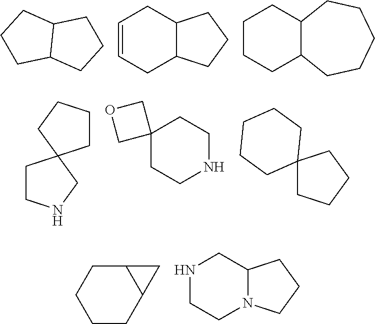

Exemplary bridged bicyclics include:

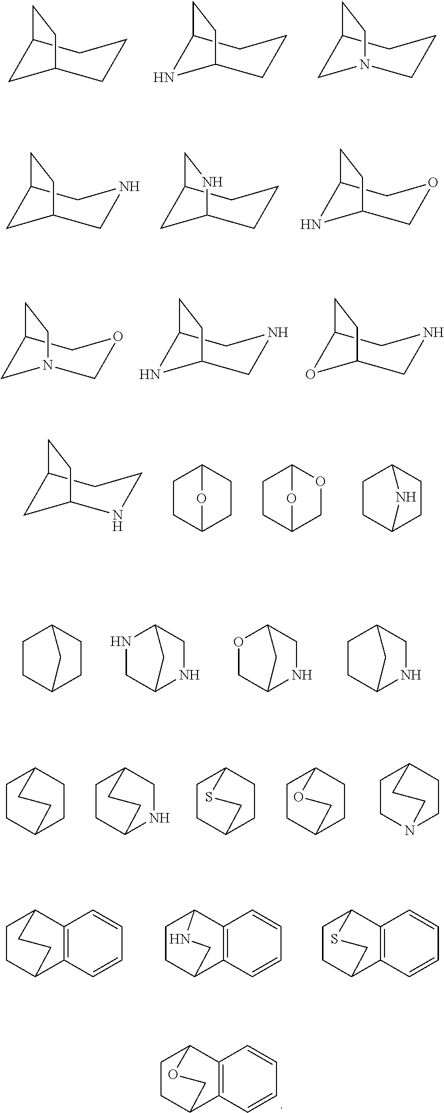

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated.” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it. For example, “phenylene” is a bivalent phenyl group when it has two groups attached to it

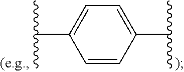 [SMILES: *c1ccc(*)cc1]

“phenylene” is a trivalent phenyl group when it has three groups attached to it

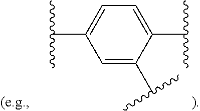 [SMILES: *c1ccc(*)c(*)c1]

The term “arylene” refers to a bivalent aryl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “heteroarylene” refers to a multivalent heteroaryl group having the appropriate number of open valences to account for groups attached to it. For example, “heteroarylene” is a bivalent heteroaryl group when it has two groups attached to it; “heteroarylene” is a trivalent heteroaryl group when it has three groups attached to it.

As used herein, the terms “heterocycle.” “heterocyclyl.” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle.” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by one or more oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it. The term “oxo-heterocyclylene” refers to a multivalent oxo-heterocyclyl group having the appropriate number of open valences to account for groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂: —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}: —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}.

Each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{●}, -(haloR^{●}), —OH, —OR^{●}, —O(haloR^{●}), —CN, —C(O)OH, —C(O)OR^{●}, —NH₂, —NHRR, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. The invention includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The terms “alkenyl” and “alkynyl” are art-recognized and refer to unsaturated aliphatic groups analogous in length and possible substitution to the alkyls described above, but that contain at least one double or triple bond respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “═O” substituent. For example, a cyclopentane substituted with an oxo group is cyclopentanone.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

term “EC₅₀” is art recognized and refers to the concentration of a compound that is required to achieve a response that is 50% of the maximum target effect relative to the baseline.

The term “Eₘₐₓ” is art recognized and refers to the concentration of a compound that is require to achieve maximal target effect.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Pyridine Carboxamide Compounds

One aspect of the invention provides pyridine carboxamide compounds. The compounds may be used in the pharmaceutical compositions and therapeutic methods described herein. Exemplary compounds are described in the following sections, along with exemplary procedures for making the compounds.

One aspect of the invention provides a compound represented by Formula I:

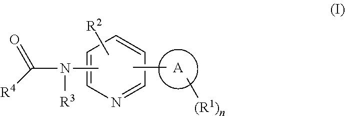

or a pharmaceutically acceptable salt thereof; wherein:
Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur, a 4-8 membered saturated or partially unsaturated monocyclic heterocyclic ring containing 1-4 heteroatoms independently selected from nitrogen, oxygen, and sulfur, a 5-6 membered monocyclic heteroaromatic ring having 1-4 heteroatoms independently selected from nitrogen, oxygen, and sulfur, a 6-11 membered saturated or partially unsaturated bicyclic carbocyclic ring, a 3-8 membered saturated or partially unsaturated monocyclic carbocyclic ring, an 8-10 membered bicyclic aromatic carbocyclic ring, or phenyl;
R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, C₁₋₄ haloaliphatic, or a cyclic group selected from a 3-8 membered saturated monocyclic carbocyclic ring or a 3-8 membered saturated monocyclic heterocyclic ring having 1-4 heteroatoms independently selected from nitrogen, oxygen, and sulfur; R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy;
R³ is hydrogen, cyclopropyl, or C₁₋₃ alkyl optionally substituted with a group selected from —OH, —OMe, —NH₂, —NH(CH₃), and —N(CH₃)₂;
R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵;
R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur;
R represents independently for each occurrence hydrogen, hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring;
n is 0, 1, 2, or 3; and
p is 0, 1, 2, 3 or 4.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

As defined generally above, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur; a 5-6 membered monocyclic heteroaromatic ring having 1-4 heteroatoms independently selected from nitrogen, oxygen, and sulfur; a 6-11 membered saturated or partially unsaturated bicyclic carbocyclic ring; a 3-8 membered saturated or partially unsaturated monocyclic carbocyclic ring; an 8-10 membered bicyclic aromatic carbocyclic ring; or phenyl.

In some embodiments, Ring A is a 6-14 membered saturated or partially unsaturated bicyclic heterocyclic ring containing 1-4 heteroatoms independently selected from nitrogen, oxygen, and sulfur. In some embodiments, Ring A is an 8-10 membered bicyclic heteroaromatic ring having 1-4 heteroatoms independently selected from nitrogen, oxygen, and sulfur. In some embodiments, Ring A is a 4-8 membered saturated or partially unsaturated monocyclic heterocyclic ring containing 1-4 heteroatoms independently selected from nitrogen, oxygen, and sulfur. In some embodiments, Ring A is a 5-6 membered monocyclic heteroaromatic ring having 1-4 heteroatoms independently selected from nitrogen, oxygen, and sulfur. In some embodiments, Ring A is a 6-11 membered saturated or partially unsaturated bicyclic carbocyclic ring; a 3-8 membered saturated or partially unsaturated monocyclic carbocyclic ring. In some embodiments, Ring A is an 8-10 membered bicyclic aromatic carbocyclic ring. In some embodiments, Ring A is phenyl.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur or a 5-6 membered monocyclic heteroaromatic ring having 1-4 heteroatoms independently selected from nitrogen, oxygen, and sulfur.

In some embodiments, Ring A is a 6-14 membered saturated or partially unsaturated bicyclic heterocyclic ring containing 1-4 heteroatoms independently selected from nitrogen, oxygen, and sulfur; or an 8-10 membered bicyclic heteroaromatic ring having 1-4 heteroatoms independently selected from nitrogen, oxygen, and sulfur.

In some embodiments, Ring A is a 4-8 membered saturated or partially unsaturated monocyclic heterocyclic ring containing 1-4 heteroatoms independently selected from nitrogen, oxygen, and sulfur or a 5-6 membered monocyclic heteroaromatic ring having 1-4 heteroatoms independently selected from nitrogen, oxygen, and sulfur.

In some embodiments, Ring A is a 6-11 membered saturated or partially unsaturated bicyclic carbocyclic ring; a 3-8 membered saturated or partially unsaturated monocyclic carbocyclic ring; an 8-10 membered bicyclic aromatic carbocyclic ring; or phenyl.

In some embodiments, Ring A is a 3-8 membered saturated or partially unsaturated monocyclic carbocyclic ring or phenyl.

In some embodiments, Ring A is a 6-11 membered saturated or partially unsaturated bicyclic carbocyclic ring; or an 8-10 membered bicyclic aromatic carbocyclic ring.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur; a 5-6 membered monocyclic heteroaromatic ring having 1-4 heteroatoms independently selected from nitrogen, oxygen, and sulfur; or a 6-11 membered saturated or partially unsaturated bicyclic carbocyclic ring; a 3-8 membered saturated or partially unsaturated monocyclic carbocyclic ring.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur; a 5-6 membered monocyclic heteroaromatic ring having 1-4 heteroatoms independently selected from oxygen, and sulfur; a 6-11 membered saturated or partially unsaturated bicyclic carbocyclic ring; a 3-8 membered saturated or partially unsaturated monocyclic carbocyclic ring; or phenyl.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur; a 6-11 membered saturated or partially unsaturated bicyclic carbocyclic ring; a 3-8 membered saturated or partially unsaturated monocyclic carbocyclic ring; or phenyl.

In some embodiments, Ring A is selected from those depicted in Table 1, below.

As defined generally above, R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ represents independently for each occurrence halo. In some embodiments, R¹ represents independently for each occurrence —OR. In some embodiments, R¹ represents independently for each occurrence —SR. In some embodiments, R¹ represents independently for each occurrence —NR₂. In some embodiments, R¹ represents independently for each occurrence —S(O)₂R. In some embodiments, R¹ represents independently for each occurrence —S(O)₂NR₂. In some embodiments, R¹ represents independently for each occurrence —S(O)R. In some embodiments, R¹ represents independently for each occurrence —S(O)NR₂. In some embodiments, R¹ represents independently for each occurrence —C(O)R. In some embodiments, R¹ represents independently for each occurrence —C(O)CR₃. In some embodiments, R¹ represents independently for each occurrence —C(O)OR. In some embodiments, R¹ represents independently for each occurrence —C(O)NR₂. In some embodiments, R¹ represents independently for each occurrence —C(O)N(R)OR. In some embodiments, R¹ represents independently for each occurrence —OC(O)R. In some embodiments, R¹ represents independently for each occurrence —OC(O)NR₂. In some embodiments, R¹ represents independently for each occurrence —O(CR₂)₃NR₂. In some embodiments, R¹ represents independently for each occurrence —N(R)C(O)OR. In some embodiments, R¹ represents independently for each occurrence —N(R)C(O)R. In some embodiments, R¹ represents independently for each occurrence —N(R)C(O)NR₂. In some embodiments, R¹ represents independently for each occurrence —N(R)C(NR)NR₂. In some embodiments, R¹ represents independently for each occurrence —NR(CR₂)₂OR. In some embodiments, R¹ represents independently for each occurrence —N(R)S(O)₂NR₂. In some embodiments, R¹ represents independently for each occurrence —N(R)S(O)₂R. In some embodiments. R¹ represents independently for each occurrence —CH₂R. In some embodiments, R¹ represents independently for each occurrence —CHR₂. In some embodiments, R¹ represents independently for each occurrence —CR₃. In some embodiments, R¹ represents independently for each occurrence —CH₂NR₂. In some embodiments, R¹ represents independently for each occurrence —CH₂NHCH₂CH₂OR. In some embodiments, R¹ represents independently for each occurrence —CH₂CH₂C(O)OR. In some embodiments. R¹ represents independently for each occurrence —CH₂C(O)NR₂. In some embodiments, R¹ represents independently for each occurrence —CH₂CH₂R. In some embodiments, R¹ represents independently for each occurrence —CH₂CH₂OR. In some embodiments, R¹ represents independently for each occurrence —NHCH₂CH₂OR. In some embodiments, R¹ represents independently for each occurrence —NHC(O)R. In some embodiments, R¹ represents independently for each occurrence —CH₂OR. In some embodiments, R¹ represents independently for each occurrence —CH₂C(O)NH₂. In some embodiments, R¹ represents independently for each occurrence —CH₂NHC(O)OCR₃. In some embodiments. R¹ represents independently for each occurrence —OCR. In some embodiments. R¹ represents independently for each occurrence C₁₋₄ aliphatic. In some embodiments, R¹ represents independently for each occurrence C₁₋₄ haloaliphatic. In some embodiments, R¹ represents independently for each occurrence a 3-8 membered saturated monocyclic carbocyclic ring. In some embodiments, R¹ represents independently for each occurrence a 3-8 membered saturated monocyclic heterocyclic ring having 1-4 heteroatoms independently selected from nitrogen, oxygen, and sulfur.

In some embodiments, R¹ represents independently for each occurrence —CN, —C(O)R, —C(O)CR₃, —C(O)OR, —C(O)NR₂, —C(O)N(R)OR, —CR₃, —CH₂NR₂—, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ represents independently for each occurrence halo, oxo, —OR, —SR, —NR₂, —S(O)₂R, —S(O)₂NR₂, —S(O)R, —S(O)NR₂, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, —NHC(O)R, or —OCR.

In some embodiments, R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, or C₁₋₄ haloaliphatic.

In some embodiments, R¹ represents independently for each occurrence halo, oxo, —CN, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ represents independently for each occurrence oxo, —OR, —OC(O)R, —OC(O)NR₂, —O(CR₂)₃NR₂, or —OCR.

In some embodiments, R¹ represents independently for each occurrence —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In some embodiments, R¹ represents independently for each occurrence halo, oxo, —OR, —C(O)R, —C(O)CR₃, —C(O)OR, —N(R)C(O)OR, —N(R)C(O)R, —N(R)C(O)NR₂, —CR₃, —CH₂OR, —CH₂C(O)NH₂, or C₁₋₄ aliphatic.

In some embodiments, R¹ represents independently for each occurrence oxo or C₁₋₄ aliphatic.

In some embodiments, R¹ represents independently for each occurrence —CH₃, —CH₂C(O)NH₂, —CH₂OCH₃, —CH₂CH₂OCH₃, —NHAc, —OH, —C(O)CH₂OH, —C(O)CH₃, —CH₂NHCH₂CH₂OH, —CH₂C(O)NH₂, —OCH₃, CH₂OCH₃, —C(O)OCH₃, —C(O)OH, —NHCH₂CH₂OH, —CH₂CH₂C(O)OCH₃, —NHS(O)₂CH₃, —C(O)NH₂, —S(O)₂CH₃, -Bn, —OC(CH₃)₂, —NHCH₂CH₂OCH₃, —F, —Cl, —Br, (O), —CH₂NH₂, —NH₂, —NHC(O)CH₃, —C(O)OCH₂CH₃, —S(O)₂NHCH₂CH₂OH, —CH₂CH₃, —N(CH₃)₂, —CH(CH₃)₂, —CF₃, —CN, —S(O)₂CH₃, —CH₂OH, —OCH₃, —CH₂N(CH₃)₂, —O(CH₂)₃N(CH₃)₂, —C(O)NH₂, -cyclohexyl, —OCF₃, —S(O)₂NH₂, —CHF₂, —CH₂NHC(O)OC(CH₃)₃, —C(CH₃)₂(OH), —CH(OH)CH₃, —O(CH₂)₂NH₂,

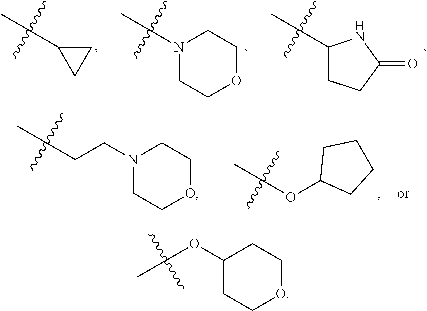

In some embodiments, R¹ represents independently for each occurrence —CH₃, —NHAc, —OH, —C(O)CH₂OH, —C(O)CH₃, —C(O)OCH₃, —C(O)OH, —C(O)NH₂, (O), —CH₂NH₂, —NH₂, —NHC(O)CH₃, —CH₂CH₃, —CH₂OH, —OCH₃, or

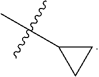 [SMILES: *C1CC1]

In some embodiments, R¹ represents independently for each occurrence —CH₃, —NHAc, —OH, (O), —OCH₃,

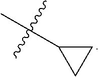 [SMILES: *C1CC1]

In some embodiments, R¹ represents independently for each occurrence —CH₃ or (O).

In some embodiments, R¹ is halo. In some embodiments, R¹ is —OR. In some embodiments, R¹ is —SR. In some embodiments, R¹ is —NR₂. In some embodiments, R¹ is —S(O)₂R. In some embodiments, R¹ is —S(O)₂NR₂. In some embodiments, R¹ is —S(O)R. In some embodiments, R¹ is —S(O)NR₂. In some embodiments, R¹ is —C(O)R. In some embodiments, R¹ is —C(O)CR₃. In some embodiments, R¹ is —C(O)OR. In some embodiments. R¹ is —C(O)NR₂. In some embodiments, R¹ is —C(O)N(R)OR. In some embodiments, R¹ is —OC(O)R. In some embodiments, R¹ is —OC(O)NR₂. In some embodiments, R¹ is —O(CR₂)₃NR₂. In some embodiments, R¹ is —N(R)C(O)OR. In some embodiments, R¹ is —N(R)C(O)R. In some embodiments, R¹ is —N(R)C(O)NR₂. In some embodiments, R¹ is —N(R)C(NR)NR₂. In some embodiments, R¹ is —NR(CR₂)₂OR. In some embodiments, R¹ is —N(R)S(O)₂NR₂. In some embodiments, R¹ is —N(R)S(O)₂R. In some embodiments, R¹ is —CR₃. In some embodiments, R¹ is —CH₂NR₂. In some embodiments, R¹ is —CH₂NHCH₂CH₂OR. In some embodiments, R¹ is —CH₂CH₂C(O)OR. In some embodiments. R¹ is —CH₂C(O)NR₂. In some embodiments. R¹ is —CH₂CH₂R. In some embodiments. R¹ is —CH₂CH₂OR. In some embodiments, R¹ is —NHCH₂CH₂OR. In some embodiments, R¹ is —NHC(O)R. In some embodiments, R¹ is —CH₂OR. In some embodiments, R¹ is —CH₂C(O)NH₂. In some embodiments, R¹ is —CH₂NHC(O)OCR₃. In some embodiments, R¹ is —OCR. In some embodiments, R¹ is C₁₋₄ aliphatic. In some embodiments, R¹ is C₁₋₄ haloaliphatic. In some embodiments, R¹ is a 3-8 membered saturated monocyclic carbocyclic ring. In some embodiments, R¹ is a 3-8 membered saturated monocyclic heterocyclic ring having 1-4 heteroatoms independently selected from nitrogen, oxygen, and sulfur.

In some embodiments, R¹ is —CN, —C(O)R, —C(O)CR₃, —C(O)OR, —C(O)NR₂, —C(O)N(R)OR, —CR₃, —CH₂NR₂, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ is halo, oxo, —OR, —SR, —NR₂, —S(O)₂R, —S(O)₂NR₂, —S(O)R, —S(O)NR₂, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, —NHC(O)R, or —OCR.

In some embodiments, R¹ is halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, or C₁₋₄ haloaliphatic.

In some embodiments, R¹ is halo, oxo, —CN, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ is oxo, —OR, —OC(O)R, —OC(O)NR₂, —O(CR₂)₃NR₂, or —OCR.

In some embodiments, R¹ is —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In some embodiments, R¹ is halo, oxo, —OR, —C(O)R, —C(O)CR₃, —C(O)OR, —N(R)C(O)OR, —N(R)C(O)R, —N(R)C(O)NR₂, —CR₃, —CH₂OR, —CH₂C(O)NH₂, or C₁₋₄ aliphatic.

In some embodiments, R¹ is oxo or C₁₋₄ aliphatic.

In some embodiments, R¹ is —CH₃, —CH₂C(O)NH₂, —CH₂OCH₃, —CH₂CH₂OCH₃, —NHAc, —OH, —C(O)CH₂OH, —C(O)CH₃, —CH₂NHCH₂CH₂OH, —CH₂C(O)NH₂, —OCH₃, CH₂OCH₃, —C(O)OCH₃, —C(O)OH, —NHCH₂CH₂OH, —CH₂CH₂C(O)OCH₃, —NHS(O)₂CH₃, —C(O)NH₂, —S(O)₂CH₃, -Bn, —OC(CH₃)₂, —NHCH₂CH₂OCH₃, —F, —Cl, —Br, (O), —CH₂NH₂, —NH₂, —NHC(O)CH₃, —C(O)OCH₂CH₃, —S(O)₂NHCH₂CH₂OH, —CH₂CH₃, —N(CH₃)₂, —CH(CH₃)₂, —CF₃, —CN, —S(O)₂CH₃, —CH₂OH, —OCH₃, —CH₂N(CH₃)₂, —O(CH₂)₃N(CH₃)₂, —C(O)NH₂, -cyclohexyl, —OCF₃, —S(O)₂NH₂, —CHF₂, —CH₂NHC(O)OC(CH₃)₃, —C(CH₃)₂(OH), —CH(OH)CH₃, —O(CH₂)₂NH₂,

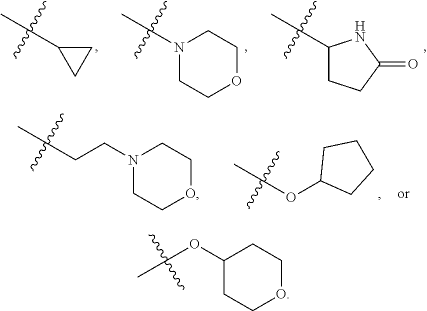

In some embodiments, R¹ is —CH₃, —NHAc, —OH, —C(O)CH₂OH, —C(O)CH₃, —C(O)OCH₃, —C(O)OH, —C(O)NH₂, (O), —CH₂NH₂, —NH₂, —NHC(O)CH₃, —CH₂CH₃, —CH₂OH, —OCH₃, or

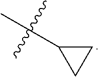 [SMILES: *C1CC1]

In some embodiments, R¹ is —CH₃, —NHAc, —OH, (O), —OCH₃, or

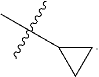 [SMILES: *C1CC1]

In some embodiments, R¹ is —CH₃ or (O).

In some embodiments, R¹ is selected from those depicted in Table 1, below.

As defined generally above, R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy.

In some embodiments, R² is halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy. In some embodiments, R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy. In some embodiments, R² is hydrogen, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy. In some embodiments, R² is hydrogen, halo, —CN, cyclopropyl, C₁₋₃ alkyl, C₁₋₃ haloalkyl, or C₁₋₃ alkoxy. In some embodiments, R² is hydrogen, halo, —OH, cyclopropyl, C₁₋₃ alkyl, C₁₋₃ haloalkyl, or C₁₋₃ alkoxy. In some embodiments, R² is hydrogen, halo, —OH, —CN, C₁₋₃ alkyl, C₁₋₃ haloalkyl, or C₁₋₃ alkoxy. In some embodiments, R² is hydrogen, halo, —OH, —CN, cyclopropyl, C₁₋₃ haloalkyl, or C₁₋₃ alkoxy. In some embodiments, R² is hydrogen, halo, —OH, —CN, cyclopropyl, C₁₋₃ alkyl, or C₁₋₃ alkoxy. In some embodiments, R² is hydrogen, halo, —OH, —CN, cyclopropyl, C₁₋₃ alkyl, or C₁₋₃ haloalkyl.

In some embodiments, R² is hydrogen, cyclopropyl, C₁₋₃ alkyl optionally substituted with a group selected from —OH, —NH₂, —NH(CH₃), and —N(CH₃)₂, or C₁₋₃ alkoxy. In some embodiments, R² is hydrogen or C₁₋₃ alkyl optionally substituted with a group selected from —OH, —NH₂, —NH(CH₃), and —N(CH₃)₂. In some embodiments, R² is hydrogen or C₁₋₃ alkyl. In some embodiments, R² unsubstituted C₁₋₃ alkyl. In some embodiments, R² is C₁₋₃ alkyl substituted with a group selected from —OH, —NH₂, —NH(CH₃), and —N(CH₃)₂.

In some embodiments, R² is hydrogen. In some embodiments, R² is —OH. In some embodiments, R² is —CN. In some embodiments, R² is cyclopropyl.

In some embodiments, R² is halo. In some embodiments, R² is Cl. F, or Br. In some embodiments, R² is Cl. In some embodiments, R² is F. In some embodiments, R² is Br.

In some embodiments, R² is C₁₋₃ alkyl. In some embodiments. R² is methyl. In some embodiments, R² is ethyl. In some embodiments, R² is i-propyl. In some embodiments, R² is n-propyl.

In some embodiments, R² is C₁₋₃ haloalkyl. In some embodiments, R² is CF₃. In some embodiments, R² is CHF₂.

In some embodiments, R² is C₁₋₃ alkoxy. In some embodiments, R² is —CH₂OCH₃. In some embodiments, R² is —CH₂OCH₃. In some embodiments, R² is —CH₂CH₂OCH₃.

In some embodiments, R² is selected from those depicted in Table 1, below.

As defined generally above, R³ is hydrogen, cyclopropyl, or C₁₋₃ alkyl optionally substituted with a group selected from —OH, OMe, —NH₂, —NH(CH₃), and —N(CH₃)₂.

In some embodiments, R³ is cyclopropyl, or C₁₋₃ alkyl optionally substituted with a group selected from —OH, —OMe, —NH₂, —NH(CH₃), and —N(CH₃)₂. In some embodiments, R³ is hydrogen, cyclopropyl, or C₁₋₃ alkyl. In some embodiments, R³ is hydrogen or C₁₋₃ alkyl optionally substituted with a group selected from —OH, —OMe, —NH₂, —NH(CH₃), and —N(CH₃)₂. In some embodiments, R³ is hydrogen, cyclopropyl, or C₁₋₃ alkyl substituted with a group selected from —OH, —OMe, —NH₂, —NH(CH₃), and —N(CH₃)₂. In some embodiments, R³ is substituted C₁₋₃ alkyl as defined above and herein, wherein the substitution occurs on the terminal carbon atom.

In some embodiments, R³ is hydrogen or C₁₋₃ alkyl optionally substituted with a group selected from —OH and OMe. In some embodiments, R³ is hydrogen or C₁₋₃ alkyl.

In some embodiments, R³ is hydrogen. In some embodiments, R³ is cyclopropyl. In some embodiments, R³ is C₁₋₃ alkyl. In some embodiments, R³ is methyl. In some embodiments, R³ is ethyl. In some embodiments, R³ is i-propyl. In some embodiments, R³ is n-propyl.

In some embodiments, R³ is C₁₋₃ alkyl substituted with a group selected from —OH, OMe, —NH₂, —NH(CH₃), and —N(CH₃)₂. In some embodiments, R³ is C₁₋₃ alkyl substituted with a group selected from —OH or OMe. In some embodiments, R³ is C₁₋₃ alkyl substituted with a group selected from —OMe, —NH₂, —NH(CH₃), or —N(CH₃)₂. In some embodiments, R³ is C₁₋₃ alkyl substituted with a group selected from —NH₂, —NH(CH₃), or —N(CH₃)₂. In some embodiments, R³ is —CH₂OCH₃. In some embodiments. R³ is —CH₂OCH₃. In some embodiments, R³ is —CH₂CH₂OCH₃.

In some embodiments, R³ is selected from those depicted in Table 1, below.

As defined generally above, R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a 3-8 membered saturated or partially unsaturated monocyclic carbocyclic ring, wherein R⁴ is substituted with p instances of R⁵. In some embodiments, R⁴ is a 7-12 membered saturated or partially unsaturated bicyclic carbocyclic ring, wherein R⁴ is substituted with p instances of R⁵. In some embodiments, R⁴ is a phenyl, wherein R⁴ is substituted with p instances of R⁵. In some embodiments, R⁴ is an 8-10 membered bicyclic aromatic carbocyclic ring wherein R⁴ is substituted with p instances of R⁵. In some embodiments, R⁴ is a 3-8 membered saturated or partially unsaturated monocyclic heterocyclic ring having 1-4 heteroatoms independently selected from nitrogen, oxygen, and sulfur, wherein R⁴ is substituted with p instances of R⁵. In some embodiments, R⁴ is a 7-12 membered saturated or partially unsaturated bicyclic heterocyclic ring having 1-4 heteroatoms independently selected from nitrogen, oxygen, and sulfur, wherein R⁴ is substituted with p instances of R⁵. In some embodiments, R⁴ is a 5-6 membered monocyclic heteroaromatic ring having 1-4 heteroatoms independently selected from nitrogen, oxygen, and sulfur, wherein R⁴ is substituted with p instances of R⁵. In some embodiments, R⁴ is and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a 3-8 membered saturated or partially unsaturated monocyclic carbocyclic ring. In some embodiments, R⁴ is a 7-12 membered saturated or partially unsaturated bicyclic carbocyclic ring. In some embodiments, R⁴ is a phenyl. In some embodiments, R⁴ is an 8-10 membered bicyclic aromatic carbocyclic ring wherein R⁴ is substituted with p instances of R⁵. In some embodiments, R⁴ is a 3-8 membered saturated or partially unsaturated monocyclic heterocyclic ring having 1-4 heteroatoms independently selected from nitrogen, oxygen, and sulfur. In some embodiments, R⁴ is a 7-12 membered saturated or partially unsaturated bicyclic heterocyclic ring having 1-4 heteroatoms independently selected from nitrogen, oxygen, and sulfur. In some embodiments, R⁴ is a 5-6 membered monocyclic heteroaromatic ring having 1-4 heteroatoms independently selected from nitrogen, oxygen, and sulfur. In some embodiments, R⁴ is and an 8-10 membered bicyclic heteroaromatic ring having 1-5 heteroatoms independently selected from nitrogen, oxygen, and sulfur.

In some embodiments, R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, wherein R⁴ is substituted with p instances of R⁵.

In some embodiments, R⁴ is a cyclic group selected a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 7-12 membered saturated or partially unsaturated bicyclic carbocyclic ring, an 8-10 membered bicyclic aromatic carbocyclic ring, a 7-12 membered saturated or partially unsaturated bicyclic heterocyclic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 3-8 membered saturated or partially unsaturated monocyclic carbocyclic ring, phenyl, an 8-10 membered bicyclic aromatic carbocyclic ring, a 5-6 membered monocyclic heteroaromatic ring having 1-4 heteroatoms independently selected from nitrogen, oxygen, and sulfur, wherein R⁴ is substituted with p instances of R⁵.

In some embodiments, R⁴ is a cyclic group selected from a 3-6 membered saturated or partially unsaturated monocyclic carbocyclic ring, a 8-10 membered saturated or partially unsaturated bicyclic carbocyclic ring, phenyl, an 8-10 membered bicyclic aromatic carbocyclic ring, a 3-6 membered saturated or partially unsaturated monocyclic heterocyclic ring having 1-4 heteroatoms independently selected from nitrogen, oxygen, and sulfur, a 8-10 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 3-6 membered saturated or partially unsaturated monocyclic carbocyclic ring, a 8-10 membered saturated or partially unsaturated bicyclic carbocyclic ring, phenyl, an 8-10 membered bicyclic aromatic carbocyclic ring, a 3-6 membered saturated or partially unsaturated monocyclic heterocyclic ring having 2-4 heteroatoms independently selected from nitrogen, oxygen, and sulfur, a 8-10 membered saturated or partially unsaturated bicyclic heterocyclic ring having 2-4 heteroatoms independently selected from nitrogen, oxygen, and sulfur, a 5-6 membered monocyclic heteroaromatic ring having 2-4 heteroatoms independently selected from nitrogen, oxygen, and sulfur, and an 8-10 membered bicyclic heteroaromatic ring having 2-5 heteroatoms independently selected from nitrogen, oxygen, and sulfur, wherein R⁴ is substituted with p instances of R⁵.

In some embodiments, R⁴ is selected from those depicted in Table 1, below.

As defined generally above, R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence halo. In certain embodiments, R⁵ represents independently for each occurrence —OR. In certain embodiments, R⁵ represents independently for each occurrence —SR. In certain embodiments, R⁵ represents independently for each occurrence —NR₂. In certain embodiments, R⁵ represents independently for each occurrence —S(O)₂R. In certain embodiments, R⁵ represents independently for each occurrence —S(O)₂NR₂. In certain embodiments, R⁵ represents independently for each occurrence —S(O)R. In certain embodiments, R⁵ represents independently for each occurrence —S(O)NR₂. In certain embodiments, R⁵ represents independently for each occurrence —C(O)R. In certain embodiments, R⁵ represents independently for each occurrence —C(O)CR₃. In certain embodiments, R⁵ represents independently for each occurrence —C(O)OR. In certain embodiments, R⁵ represents independently for each occurrence —C(O)NR₂. In certain embodiments, R⁵ represents independently for each occurrence —C(O)N(R)OR. In certain embodiments, R⁵ represents independently for each occurrence —OC(O)R. In certain embodiments, R⁵ represents independently for each occurrence —OC(O)NR₂. In certain embodiments, R⁵ represents independently for each occurrence —O(CR₂)₃NR₂. In certain embodiments, R⁵ represents independently for each occurrence —N(R)C(O)OR. In certain embodiments, R⁵ represents independently for each occurrence —N(R)C(O)R. In certain embodiments, R⁵ represents independently for each occurrence —N(R)C(O)NR₂. In certain embodiments. R⁵ represents independently for each occurrence —N(R)C(NR)NR₂. In certain embodiments, R⁵ represents independently for each occurrence-NR(CR₂)₂OR. In certain embodiments, R⁵ represents independently for each occurrence —N(R)S(O)₂NR₂. In certain embodiments, R⁵ represents independently for each occurrence —N(R)S(O)₂R. In certain embodiments, R⁵ represents independently for each occurrence —CH₂R. In certain embodiments, R⁵ represents independently for each occurrence —CHR₂. In certain embodiments, R⁵ represents independently for each occurrence —CR₃. In certain embodiments, R⁵ represents independently for each occurrence —CH₂NR₂. In certain embodiments, R⁵ represents independently for each occurrence —CH₂NHCH₂CH₂OR. In certain embodiments, R⁵ represents independently for each occurrence —CH₂CH₂C(O)OR. In certain embodiments, R⁵ represents independently for each occurrence —CH₂C(O)NR₂. In certain embodiments, R⁵ represents independently for each occurrence —CH₂CH₂R, In certain embodiments, R⁵ represents independently for each occurrence —CH₂CH₂OR. In certain embodiments, R⁵ represents independently for each occurrence —NHCH₂CH₂OR. In certain embodiments, R⁵ represents independently for each occurrence —NHC(O)R. In certain embodiments, R⁵ represents independently for each occurrence —CH₂OR. In certain embodiments, R⁵ represents independently for each occurrence —CH₂NHC(O)OCR₃. In certain embodiments, R⁵ represents independently for each occurrence a C₁₋₃ alkyl group. In certain embodiments, R⁵ represents independently for each occurrence a C₁₋₃ haloalkyl group. In certain embodiments, R⁵ represents independently for each occurrence a C₁₋₃ heteroalkyl group having 1-2 heteroatoms independently selected from nitrogen, oxygen, and sulfur. In certain embodiments, R⁵ represents independently for each occurrence a 3-6 membered saturated or partially unsaturated monocyclic carbocyclic ring. In certain embodiments, R⁵ represents independently for each occurrence phenyl. In certain embodiments, R⁵ represents independently for each occurrence a 5-6 membered monocyclic heteroaromatic ring having 1-4 heteroatoms independently selected from nitrogen, oxygen, and sulfur. In certain embodiments, R⁵ represents independently for each occurrence a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence oxo, —OR, —OC(O)R, —OC(O)NR₂, or —O(CR₂)₃NR₂.

In certain embodiments, R⁵ represents independently for each occurrence —CN, —C(O)R, —C(O)CR₃, —C(O)OR, —C(O)NR₂, —C(O)N(R)OR, —CR₃, —CH₂NR₂, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In certain embodiments, R⁵ represents independently for each occurrence a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, or a C₁₋₃ haloalkyl group.

In certain embodiments, R⁵ represents independently for each occurrence halo, oxo, —CN, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence halo, oxo, a C₁₋₃ alkyl group, a C₁₋₃ haloalkyl group, or a C₁₋₃ heteroalkyl group having 1-2 heteroatoms independently selected from nitrogen, oxygen, and sulfur.

In certain embodiments, R⁵ is halo. In certain embodiments, R⁵ is —OR. In certain embodiments, R⁵ is —SR. In certain embodiments, R⁵ is —NR₂. In certain embodiments, R⁵ is —S(O)₂R.

In certain embodiments, R⁵ is —S(O)₂NR₂. In certain embodiments, R⁵ is —S(O)R. In certain embodiments, R⁵ is —S(O)NR₂. In certain embodiments, R⁵ is —C(O)R. In certain embodiments, R⁵ is —C(O)CR₃. In certain embodiments, R⁵ is —C(O)OR. In certain embodiments, R⁵ is —C(O)NR₂. In certain embodiments, R⁵ is —C(O)N(R)OR. In certain embodiments, R⁵ is —OC(O)R. In certain embodiments, R⁵ is —OC(O)NR₂. In certain embodiments, R⁵ is —O(CR₂)₃NR₂. In certain embodiments, R⁵ is —N(R)C(O)OR. In certain embodiments, R⁵ is —N(R)C(O)R. In certain embodiments, R⁵ is —N(R)C(O)NR₂. In certain embodiments, R⁵ is —N(R)C(NR)NR₂. In certain embodiments, R⁵ is —NR(CR₂)₂OR. In certain embodiments, R⁵ is —N(R)S(O)₂NR₂. In certain embodiments, R⁵ is —N(R)S(O)₂R. In certain embodiments, R⁵ is —CH₂R. In certain embodiments, R⁵ is —CHR₂. In certain embodiments, R⁵ is —CR₃. In certain embodiments, R⁵ is —CH₂NR₂. In certain embodiments, R⁵ is —CH₂NHCH₂CH₂OR. In certain embodiments, R⁵ is —CH₂CH₂C(O)OR. In certain embodiments, R⁵ is —CH₂C(O)NR₂. In certain embodiments, R⁵ is —CH₂CH₂R, In certain embodiments, R⁵ is —CH₂CH₂OR. In certain embodiments, R⁵ is —NHCH₂CH₂OR. In certain embodiments, R is —NHC(O)R. In certain embodiments, R⁵ is —CH₂OR. In certain embodiments, R⁵ is —CH₂NHC(O)OCR₃. In certain embodiments, R⁵ isa C₁₋₃ alkyl group. In certain embodiments, R⁵ is a C₁₋₃ haloalkyl group. In certain embodiments, R⁵ is a C₁₋₃ heteroalkyl group having 1-2 heteroatoms independently selected from nitrogen, oxygen, and sulfur. In certain embodiments, R⁵ is a 3-6 membered saturated or partially unsaturated monocyclic carbocyclic ring. In certain embodiments, R⁵ is phenyl. In certain embodiments, R⁵ is a 5-6 membered monocyclic heteroaromatic ring having 1-4 heteroatoms independently selected from nitrogen, oxygen, and sulfur. In certain embodiments, R⁵ is a 3-6 membered saturated or partially unsaturated monocyclic heterocyclic ring having 1-4 heteroatoms independently selected from nitrogen, oxygen, and sulfur.

In certain embodiments, R⁵ is oxo, —OR, —OC(O)R, —OC(O)NR₂, or —O(CR₂)₃NR₂.

In certain embodiments, R⁵ is —CN, —C(O)R, —C(O)CR₃, —C(O)OR, —C(O)NR₂, —C(O)N(R)OR, —CR₃, —CH₂NR₂—, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ is —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In certain embodiments, R⁵ is a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ is halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂—, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, or a C₁₋₃ haloalkyl group.

In certain embodiments, R⁵ is halo, oxo, —CN, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ is halo, oxo, a C₁₋₃ alkyl group, a C₁₋₃ haloalkyl group, or a C₁₋₃ heteroalkyl group having 1-2 heteroatoms independently selected from nitrogen, oxygen, and sulfur.

In some embodiments, R⁵ is selected from those depicted in Table 1, below.

As defined generally above, R represents independently for each occurrence hydrogen, hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence halo. In certain embodiments, R represents independently for each occurrence optionally substituted C₁₋₆ aliphatic. In certain embodiments, R represents independently for each occurrence optionally substituted phenyl. In certain embodiments, R represents independently for each occurrence optionally substituted 3-7 membered saturated or partially unsaturated monocyclic heterocyclic ring having 1-4 heteroatoms independently selected from nitrogen, oxygen, and sulfur. In certain embodiments, R represents independently for each occurrence optionally substituted 5-6 membered monocyclic heteroaryl ring having 1-4 heteroatoms independently selected from nitrogen, oxygen, and sulfur. In certain embodiments, R represents independently for each occurrence optionally substituted 3-7 membered saturated or partially unsaturated monocyclic carbocyclic ring.

In certain embodiments, R represents independently for each occurrence halo. In certain embodiments, R represents independently for each occurrence C₁₋₆ aliphatic. In certain embodiments. R represents independently for each occurrence phenyl. In certain embodiments, R represents independently for each occurrence 3-7 membered saturated or partially unsaturated monocyclic heterocyclic ring having 1-4 heteroatoms independently selected from nitrogen, oxygen, and sulfur. In certain embodiments, R represents independently for each occurrence 5-6 membered monocyclic heteroaryl ring having 1-4 heteroatoms independently selected from nitrogen, oxygen, and sulfur. In certain embodiments, R represents independently for each occurrence 3-7 membered saturated or partially unsaturated monocyclic carbocyclic ring.

In certain embodiments, R represents independently for each occurrence hydrogen, hydroxyl, halo, or a group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence hydrogen, hydroxyl, halo, or optionally substituted C₁₋₆ aliphatic.

In certain embodiments, R represents independently for each occurrence an optionally substituted group selected from phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence hydrogen, hydroxyl, halo, or an optionally substituted group selected from C₁₋₃ aliphatic, phenyl, a 3-6 membered saturated or partially unsaturated monocyclic heterocyclic ring having 1-3 heteroatoms independently selected from nitrogen, oxygen, and sulfur, a 5-6 membered monocyclic heteroaryl ring having 1-3 heteroatoms independently selected from nitrogen, oxygen, and sulfur, and a 3-6 membered saturated or partially unsaturated monocyclic carbocyclic ring.

In certain embodiments, R represents independently for each occurrence hydrogen, hydroxyl, Cl, F, —CH₂OH, —CH₂CH₂OH, methyl, ethyl, i-propyl, n-propyl, cyclopropyl, or phenyl.

In certain embodiments, R is halo. In certain embodiments, R is optionally substituted C₁₋₆ aliphatic. In certain embodiments, R is optionally substituted phenyl. In certain embodiments, R is optionally substituted 3-7 membered saturated or partially unsaturated monocyclic heterocyclic ring having 1-4 heteroatoms independently selected from nitrogen, oxygen, and sulfur. In certain embodiments, R is optionally substituted 5-6 membered monocyclic heteroaryl ring having 1-4 heteroatoms independently selected from nitrogen, oxygen, and sulfur. In certain embodiments, R is optionally substituted 3-7 membered saturated or partially unsaturated monocyclic carbocyclic ring.

In certain embodiments, R is halo. In certain embodiments, R is C₁₋₆ aliphatic. In certain embodiments, R is phenyl. In certain embodiments, R is 3-7 membered saturated or partially unsaturated monocyclic heterocyclic ring having 1-4 heteroatoms independently selected from nitrogen, oxygen, and sulfur. In certain embodiments, R is 5-6 membered monocyclic heteroaryl ring having 1-4 heteroatoms independently selected from nitrogen, oxygen, and sulfur. In certain embodiments, R is 3-7 membered saturated or partially unsaturated monocyclic carbocyclic ring.

In certain embodiments, R is hydrogen, hydroxyl, halo, or a group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R is hydrogen, hydroxyl, halo, or optionally substituted C₁₋₆ aliphatic.

In certain embodiments, R is an optionally substituted group selected from phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R is hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R is hydrogen, hydroxyl, halo, or an optionally substituted group selected from C₁₋₃ aliphatic, phenyl, a 3-6 membered saturated or partially unsaturated monocyclic heterocyclic ring having 1-3 heteroatoms independently selected from nitrogen, oxygen, and sulfur, a 5-6 membered monocyclic heteroaryl ring having 1-3 heteroatoms independently selected from nitrogen, oxygen, and sulfur, and a 3-6 membered saturated or partially unsaturated monocyclic carbocyclic ring.

In certain embodiments, R is hydrogen, hydroxyl, Cl, F, —CH₂OH, —CH₂CH₂OH, methyl, ethyl, i-propyl, n-propyl, cyclopropyl, or phenyl.

In some embodiments, R is selected from those depicted in Table 1, below.

As defined generally above, n is 0, 1, 2, or 3. In certain embodiments, n is 1, 2, or 3. In certain embodiments, n is 0, 2, or 3. In certain embodiments, n is 0, 1, or 3. In certain embodiments, n is 0, 1, or 2. In certain embodiments, n is 2 or 3. In certain embodiments, n is 0 or 1. In certain embodiments, n is 0 or 3. In certain embodiments, n is 0 or 2. In certain embodiments, n is 1 or 2. In certain embodiments, n is 1 or 3. In certain embodiments, n is 0. In certain embodiments, n is 1. In certain embodiments, n is 2. In certain embodiments, n is 3.

As defined generally above, p is 0, 1, 2, 3 or 4. In certain embodiments, p is 1, 2, 3 or 4. In certain embodiments, p is 0, 2, 3 or 4. In certain embodiments, p is 0, 1, 3 or 4. In certain embodiments, p is 0, 1, 2, or 4. In certain embodiments, p is 0, 1, 2, or 3. In certain embodiments, p is 2, 3 or 4. In certain embodiments, p is 0, 3 or 4. In certain embodiments, p is 0, 1, or 4. In certain embodiments, p is 0, 1, or 2. In certain embodiments, p is 1, 3 or 4. In certain embodiments, p is 1, 2, or 4. In certain embodiments, p is 1, 2, or 3. In certain embodiments, p is 0, 2 or 4. In certain embodiments, p is 0, 2 or 3. In certain embodiments, p is 0, 1 or 3. In certain embodiments, p is 3 or 4. In certain embodiments, p is 0 or 4. In certain embodiments, p is 0 or 1. In certain embodiments, p is 1 or 4. In certain embodiments, p is 1 or 2. In certain embodiments, p is 0 or 2. In certain embodiments, p is 0 or 3. In certain embodiments, p is 1 or 3. In certain embodiments, p is 2 or 3. In certain embodiments, p is 2 or 4. In certain embodiments, p is 0. In certain embodiments, p is 1. In certain embodiments, p is 2. In certain embodiments, p is 3. In certain embodiments, p is 4.

In some embodiments, the present invention provides a compound of Formulae I-A, I-B, or I-C:

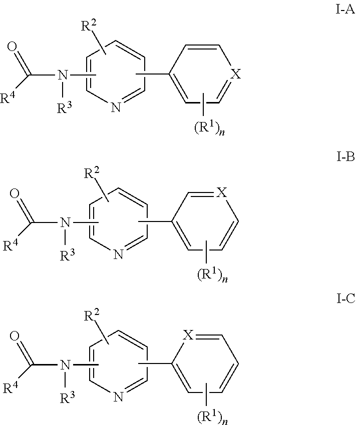

or a pharmaceutically acceptable salt thereof, wherein each of Ring A, R¹, R², R³, R⁴, R⁵, R, n, and p is as defined above and described in embodiments herein, both singly and in combination; and X is nitrogen or carbon.

In certain embodiments, X is nitrogen.

In some embodiments, the present invention provides a compound of Formulae I-D:

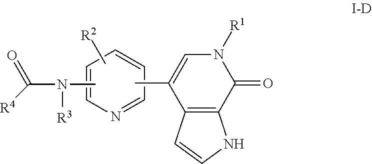

or a pharmaceutically acceptable salt thereof, wherein each of Ring A, R¹, R², R³, R⁴, R⁵, R, n, and p is as defined above and described in embodiments herein, both singly and in combination.

In certain embodiments, R¹ is —CH₃, R² is —CH₃, and R³ is —H.

One aspect of the invention provides a compound represented by Formula II:

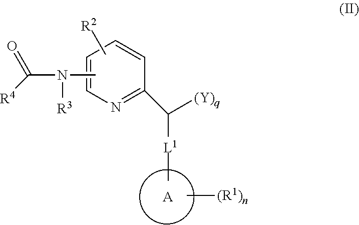

or a pharmaceutically acceptable salt thereof; wherein:
Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur, a 4-8 membered saturated or partially unsaturated monocyclic heterocyclic ring containing 1-4 heteroatoms independently selected from nitrogen, oxygen, and sulfur, a 5-6 membered monocyclic heteroaromatic ring having 1-4 heteroatoms independently selected from nitrogen, oxygen, and sulfur, a 6-11 membered saturated or partially unsaturated bicyclic carbocyclic ring, a 3-8 membered saturated or partially unsaturated monocyclic carbocyclic ring, an 8-10 membered bicyclic aromatic carbocyclic ring, or phenyl;
R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, C₁₋₄ haloaliphatic, or a cyclic group selected from a 3-8 membered saturated monocyclic carbocyclic ring or a 3-8 membered saturated monocyclic heterocyclic ring having 1-4 heteroatoms independently selected from nitrogen, oxygen, and sulfur;
R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy;
R³ is hydrogen, cyclopropyl, or C₁₋₃ alkyl optionally substituted with a group selected from —OH, —OMe, —NH₂, —NH(CH₃), and —N(CH₃)₂;
R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵;
R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur;
Y represents independently for each occurrence —R, oxo, —OR, —NR₂, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur;
L¹ is a covalent bond, —OCH₂—, —O—, —OCH₂CH₂—, —NHCH₂—, —NHCH₂CH₂—, or —NH—;
R represents independently for each occurrence hydrogen, hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring;
n is 0, 1, 2, or 3;
p is 0, 1, 2, 3 or 4; and
q is 0, 1 or 2.

The definitions of variables in Formula II above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II.

As defined generally above, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur: a 4-8 membered saturated or partially unsaturated monocyclic heterocyclic ring containing 1-4 heteroatoms independently selected from nitrogen, oxygen, and sulfur; a 5-6 membered monocyclic heteroaromatic ring having 1-4 heteroatoms independently selected from nitrogen, oxygen, and sulfur; a 6-11 membered saturated or partially unsaturated bicyclic carbocyclic ring; a 3-8 membered saturated or partially unsaturated monocyclic carbocyclic ring; an 8-10 membered bicyclic aromatic carbocyclic ring; or phenyl.

In some embodiments, Ring A is a 6-14 membered saturated or partially unsaturated bicyclic heterocyclic ring containing 1-4 heteroatoms independently selected from nitrogen, oxygen, and sulfur. In some embodiments, Ring A is an 8-10 membered bicyclic heteroaromatic ring having 1-4 heteroatoms independently selected from nitrogen, oxygen, and sulfur. In some embodiments, Ring A is a 4-8 membered saturated or partially unsaturated monocyclic heterocyclic ring containing 1-4 heteroatoms independently selected from nitrogen, oxygen, and sulfur. In some embodiments, Ring A is a 5-6 membered monocyclic heteroaromatic ring having 1-4 heteroatoms independently selected from nitrogen, oxygen, and sulfur. In some embodiments, Ring A is a 6-11 membered saturated or partially unsaturated bicyclic carbocyclic ring; a 3-8 membered saturated or partially unsaturated monocyclic carbocyclic ring. In some embodiments, Ring A is an 8-10 membered bicyclic aromatic carbocyclic ring. In some embodiments, Ring A is phenyl.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur or a 5-6 membered monocyclic heteroaromatic ring having 1-4 heteroatoms independently selected from nitrogen, oxygen, and sulfur.

In some embodiments, Ring A is a 6-14 membered saturated or partially unsaturated bicyclic heterocyclic ring containing 1-4 heteroatoms independently selected from nitrogen, oxygen, and sulfur; or an 8-10 membered bicyclic heteroaromatic ring having 1-4 heteroatoms independently selected from nitrogen, oxygen, and sulfur.

In some embodiments, Ring A is a 4-8 membered saturated or partially unsaturated monocyclic heterocyclic ring containing 1-4 heteroatoms independently selected from nitrogen, oxygen, and sulfur or a 5-6 membered monocyclic heteroaromatic ring having 1-4 heteroatoms independently selected from nitrogen, oxygen, and sulfur.

In some embodiments, Ring A is a 6-11 membered saturated or partially unsaturated bicyclic carbocyclic ring; a 3-8 membered saturated or partially unsaturated monocyclic carbocyclic ring; an 8-10 membered bicyclic aromatic carbocyclic ring; or phenyl.

In some embodiments, Ring A a 3-8 membered saturated or partially unsaturated monocyclic carbocyclic ring or phenyl.

In some embodiments, Ring A is a 6-11 membered saturated or partially unsaturated bicyclic carbocyclic ring; or an 8-10 membered bicyclic aromatic carbocyclic ring.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur; a 5-6 membered monocyclic heteroaromatic ring having 1-4 heteroatoms independently selected from nitrogen, oxygen, and sulfur; or a 6-11 membered saturated or partially unsaturated bicyclic carbocyclic ring; a 3-8 membered saturated or partially unsaturated monocyclic carbocyclic ring.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur: a 4-8 membered saturated or partially unsaturated monocyclic heterocyclic ring containing 1-4 heteroatoms independently selected from nitrogen, oxygen, and sulfur; a 5-6 membered monocyclic heteroaromatic ring having 1-4 heteroatoms independently selected from oxygen, and sulfur: a 6-11 membered saturated or partially unsaturated bicyclic carbocyclic ring; a 3-8 membered saturated or partially unsaturated monocyclic carbocyclic ring; or phenyl.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur; a 6-11 membered saturated or partially unsaturated bicyclic carbocyclic ring; a 3-8 membered saturated or partially unsaturated monocyclic carbocyclic ring; or phenyl.

In some embodiments, Ring A is selected from those depicted in Table 1, below.

As defined generally above, R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ represents independently for each occurrence halo. In some embodiments, R¹ represents independently for each occurrence —OR. In some embodiments, R¹ represents independently for each occurrence —SR. In some embodiments, R¹ represents independently for each occurrence —NR₂. In some embodiments, R¹ represents independently for each occurrence —S(O)₂R. In some embodiments, R¹ represents independently for each occurrence —S(O)₂NR₂. In some embodiments. R¹ represents independently for each occurrence —S(O)R. In some embodiments, R¹ represents independently for each occurrence —S(O)NR₂. In some embodiments, R¹ represents independently for each occurrence —C(O)R. In some embodiments, R¹ represents independently for each occurrence —C(O)CR₃. In some embodiments, R¹ represents independently for each occurrence —C(O)OR. In some embodiments. R¹ represents independently for each occurrence —C(O)NR₂. In some embodiments. R¹ represents independently for each occurrence —C(O)N(R)OR. In some embodiments, R¹ represents independently for each occurrence —OC(O)R. In some embodiments, R¹ represents independently for each occurrence —OC(O)NR₂. In some embodiments, R¹ represents independently for each occurrence —O(CR₂)₃NR₂. In some embodiments, R¹ represents independently for each occurrence —N(R)C(O)OR. In some embodiments, R¹ represents independently for each occurrence —N(R)C(O)R. In some embodiments, R¹ represents independently for each occurrence —N(R)C(O)NR₂. In some embodiments, R¹ represents independently for each occurrence —N(R)C(NR)NR₂. In some embodiments, R¹ represents independently for each occurrence —NR(CR₂)₂OR. In some embodiments, R¹ represents independently for each occurrence —N(R)S(O)₂NR₂. In some embodiments, R¹ represents independently for each occurrence —N(R)S(O)₂R. In some embodiments, R¹ represents independently for each occurrence —CH₂R. In some embodiments, R¹ represents independently for each occurrence —CHR₂. In some embodiments, R¹ represents independently for each occurrence —CR₃. In some embodiments, R¹ represents independently for each occurrence —CH₂NR₂. In some embodiments, R¹ represents independently for each occurrence —CH₂NHCH₂CH₂OR. In some embodiments, R¹ represents independently for each occurrence —CH₂CH₂C(O)OR. In some embodiments, R¹ represents independently for each occurrence —CH₂C(O)NR₂. In some embodiments, R¹ represents independently for each occurrence —CH₂CH₂R. In some embodiments, R¹ represents independently for each occurrence —CH₂CH₂OR. In some embodiments, R¹ represents independently for each occurrence —NHCH₂CH₂OR. In some embodiments, R¹ represents independently for each occurrence —NHC(O)R. In some embodiments, R¹ represents independently for each occurrence —CH₂OR. In some embodiments, R¹ represents independently for each occurrence —CH₂C(O)NH₂. In some embodiments, R¹ represents independently for each occurrence —CH₂NHC(O)OCR₃. In some embodiments, R¹ represents independently for each occurrence —OCR. In some embodiments, R¹ represents independently for each occurrence C₁₋₄ aliphatic. In some embodiments, R¹ represents independently for each occurrence C₁₋₄ haloaliphatic. In some embodiments, R¹ represents independently for each occurrence a 3-8 membered saturated monocyclic carbocyclic ring. In some embodiments, R¹ represents independently for each occurrence a 3-8 membered saturated monocyclic heterocyclic ring having 1-4 heteroatoms independently selected from nitrogen, oxygen, and sulfur.

In some embodiments, R¹ represents independently for each occurrence —CN, —C(O)R, —C(O)CR₃, —C(O)OR, —C(O)NR₂, —C(O)N(R)OR, —CR₃, —CH₂NR₂—, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ represents independently for each occurrence halo, oxo, —OR, —SR, —NR₂, —S(O)₂R, —S(O)₂NR₂, —S(O)R, —S(O)NR₂, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, —NHC(O)R, or —OCR.

In some embodiments, R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, or C₁₋₄ haloaliphatic.

In some embodiments, R¹ represents independently for each occurrence halo, oxo, —CN, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ represents independently for each occurrence oxo, —OR, —OC(O)R, —OC(O)NR₂, —O(CR₂)₃NR₂, or —OCR.

In some embodiments, R¹ represents independently for each occurrence —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In some embodiments, R¹ represents independently for each occurrence halo, oxo, —OR, —C(O)R, —C(O)CR₃, —C(O)OR, —N(R)C(O)OR, —N(R)C(O)R, —N(R)C(O)NR₂, —CR₃, —CH₂OR, —CH₂C(O)NH₂, or C₁₋₄ aliphatic.

In some embodiments, R¹ represents independently for each occurrence oxo or C₁ aliphatic.

In some embodiments, R¹ represents independently for each occurrence —CH₃, —CH₂C(O)NH₂, —CH₂OCH₃, —CH₂CH₂OCH₃, —NHAc, —OH, —C(O)CH₂OH, —C(O)CH₃, —CH₂NHCH₂CH₂OH, —CH₂C(O)NH₂, —OCH₃, CH₂OCH₃, —C(O)OCH₃, —C(O)OH, —NHCH₂CH₂OH, —CH₂CH₂C(O)OCH₃, —NHS(O)₂CH₃, —C(O)NH₂, —S(O)₂CH₃, -Bn, —OC(CH₃)₂, —NHCH₂CH₂OCH₃, —F, —Cl, —Br, (O), —CH₂NH₂, —NH₂, —NHC(O)CH₃, —C(O)OCH₂CH₃, —S(O)₂NHCH₂CH₂OH, —CH₂CH₃, —N(CH₃)₂, —CH(CH₃)₂, —CF₃, —CN, —S(O)₂CH₃, —CH₂OH, —OCH₃, —CH₂N(CH₃)₂, —O(CH₂)₃N(CH₃)₂, —C(O)NH₂, -cyclohexyl, —OCF₃, —S(O)₂NH₂, —CHF₂, —CH₂NHC(O)OC(CH₃)₃, —C(CH₃)₂(OH), —CH(OH)CH₃, —O(CH₂)₂NH₂,

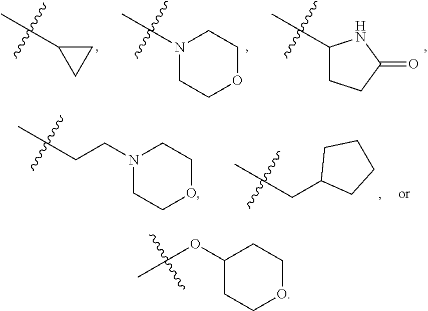

In some embodiments, R¹ represents independently for each occurrence —CH₃, —NHAc, —OH, —C(O)CH₂OH, —C(O)CH₃, —C(O)OCH₃, —C(O)OH, —C(O)NH₂, (O), —CH₂NH₂, —NH₂, —NHC(O)CH₃, —CH₂CH₃, —CH₂OH, —OCH₃, or

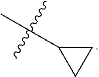 [SMILES: *C1CC1]

In some embodiments, R¹ represents independently for each occurrence —CH₃, —NHAc, —OH, (O), —OCH₃, or

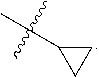 [SMILES: *C1CC1]

In some embodiments, R¹ represents independently for each occurrence —CH₃ or (O).

In some embodiments, R¹ is halo. In some embodiments, R¹ is —OR. In some embodiments, R¹ is —SR. In some embodiments, R¹ is —NR₂. In some embodiments, R¹ is —S(O)₂R. In some embodiments, R¹ is —S(O)₂NR₂. In some embodiments, R¹ is —S(O)R. In some embodiments, R¹ is —S(O)NR₂. In some embodiments, R¹ is —C(O)R. In some embodiments, R¹ is —C(O)CR₃. In some embodiments, R¹ is —C(O)OR. In some embodiments, R¹ is —C(O)NR₂. In some embodiments, R¹ is —C(O)N(R)OR. In some embodiments, R¹ is —OC(O)R. In some embodiments, R¹ is —OC(O)NR₂. In some embodiments, R¹ is —O(CR₂)₃NR₂. In some embodiments, R¹ is —N(R)C(O)OR. In some embodiments, R¹ is —N(R)C(O)R. In some embodiments, R¹ is —N(R)C(O)NR₂. In some embodiments, R¹ is —N(R)C(NR)NR₂. In some embodiments, R¹ is —NR(CR₂)₂OR. In some embodiments, R¹ is —N(R)S(O)₂NR₂. In some embodiments, R¹ is —N(R)S(O)₂R. In some embodiments, R¹ is —CR₃. In some embodiments. R¹ is —CH₂NR₂. In some embodiments, R¹ is —CH₂NHCH₂CH₂OR. In some embodiments, R¹ is —CH₂CH₂C(O)OR. In some embodiments. R¹ is —CH₂C(O)NR₂. In some embodiments, R¹ is —CH₂CH₂R. In some embodiments, R¹ is —CH₂CH₂OR. In some embodiments, R¹ is —NHCH₂CH₂OR. In some embodiments, R¹ is —NHC(O)R. In some embodiments, R¹ is —CH₂OR. In some embodiments, R¹ is —CH₂C(O)NH₂. In some embodiments, R¹ is —CH₂NHC(O)OCR₃. In some embodiments, R¹ is —OCR. In some embodiments, R¹ is C₁₋₄ aliphatic. In some embodiments, R¹ is C₁₋₄ haloaliphatic. In some embodiments, R¹ is a 3-8 membered saturated monocyclic carbocyclic ring. In some embodiments, R¹ is a 3-8 membered saturated monocyclic heterocyclic ring having 1-4 heteroatoms independently selected from nitrogen, oxygen, and sulfur.

In some embodiments, R¹ is —CN, —C(O)R, —C(O)CR₃, —C(O)OR, —C(O)NR₂, —C(O)N(R)OR, —CR₃, —CH₂NR₂, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ is halo, oxo, —OR, —SR, —NR₂, —S(O)₂R, —S(O)₂NR₂, —S(O)R, —S(O)NR₂, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, —NHC(O)R, or —OCR.

In some embodiments, R¹ is halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, or C₁₋₄ haloaliphatic.

In some embodiments, R¹ is halo, oxo, —CN, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ is oxo, —OR, —OC(O)R, —OC(O)NR₂, —O(CR₂)₃NR₂, or —OCR.

In some embodiments, R¹ is —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In some embodiments, R¹ is halo, oxo, —OR, —C(O)R, —C(O)CR₃, —C(O)OR, —N(R)C(O)OR, —N(R)C(O)R, —N(R)C(O)NR₂, —CR₃, —CH₂OR, —CH₂C(O)NH₂, or C₁₋₄ aliphatic.

In some embodiments, R¹ is oxo or C₁₋₄ aliphatic.

In some embodiments, R¹ is —CH₃, —CH₂C(O)NH₂, —CH₂OCH₃, —CH₂CH₂OCH₃, —NHAc, —OH, —C(O)CH₂OH, —C(O)CH₃, —CH₂NHCH₂CH₂OH, —CH₂C(O)NH₂, —OCH₃, CH₂OCH₃, —C(O)OCH₃, —C(O)OH, —NHCH₂CH₂OH, —CH₂CH₂C(O)OCH₃, —NHS(O)₂CH₃, —C(O)NH₂, —S(O)₂CH₃, -Bn, —OC(CH₃)₂, —NHCH₂CH₂OCH₃, —F, —Cl, —Br, (O), —CH₂NH₂, —NH₂, —NHC(O)CH₃, —C(O)OCH₂CH₃, —S(O)₂NHCH₂CH₂OH, —CH₂CH₃, —N(CH₃)₂, —CH(CH₃)₂, —CF₃, —CN, —S(O)₂CH₃, —CH₂OH, —OCH₃, —CH₂N(CH₃)₂, —O(CH₂)₃N(CH₃)₂, —C(O)NH₂, -cyclohexyl, —OCF₃, —S(O)₂NH₂, —CHF₂, —CH₂NHC(O)OC(CH₃)₃, —C(CH₃)₂(OH), —CH(OH)CH₃, —O(CH₂)NH₂,

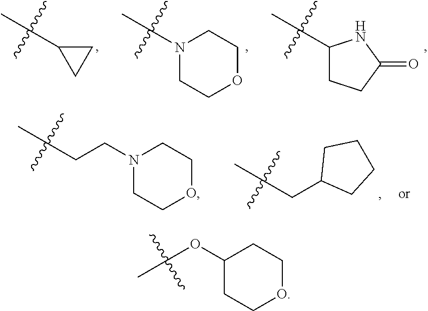

In some embodiments, R¹ is —CH₃, —NHAc, —OH, —C(O)CH₂OH, —C(O)CH₃, —C(O)OCH₃, —C(O)OH, —C(O)NH₂, (O), —CH₂NH₂, —NH₂, —NHC(O)CH₃, —CH₂CH₃, —CH₂OH, —OCH₃, or

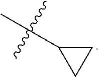 [SMILES: *C1CC1]

In some embodiments, R¹ is —CH₃, —NHAc, —OH, (O), —OCH₃, or

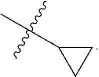 [SMILES: *C1CC1]

In some embodiments, R¹ is —CH₃ or (O).

In some embodiments, R¹ is selected from those depicted in Table 1, below.

As defined generally above, R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy.

In some embodiments, R² is halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy. In some embodiments, R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy. In some embodiments, R² is hydrogen, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy. In some embodiments, R² is hydrogen, halo, —CN, cyclopropyl, C₁₋₃ alkyl, C₁₋₃ haloalkyl, or C₁₋₃ alkoxy. In some embodiments, R² is hydrogen, halo, —OH, cyclopropyl, C₁₋₃ alkyl, C₁₋₃ haloalkyl, or C₁₋₃ alkoxy. In some embodiments, R² is hydrogen, halo, —OH, —CN, C₁₋₃ alkyl, C₁₋₃ haloalkyl, or C₁₋₃ alkoxy. In some embodiments, R² is hydrogen, halo, —OH, —CN, cyclopropyl, C₁₋₃ haloalkyl, or C₁₋₃ alkoxy. In some embodiments, R² is hydrogen, halo, —OH, —CN, cyclopropyl, C₁₋₃ alkyl, or C₁₋₃ alkoxy. In some embodiments, R² is hydrogen, halo, —OH, —CN, cyclopropyl, C₁₋₃ alkyl, or C₁₋₃ haloalkyl.

In some embodiments, R² is hydrogen, cyclopropyl. C₁₋₃ alkyl optionally substituted with a group selected from —OH, —NH₂, —NH(CH₃), and —N(CH₃)₂, or C₁₋₃ alkoxy. In some embodiments, R² is hydrogen or C₁₋₃ alkyl optionally substituted with a group selected from —OH, —NH₂, —NH(CH₃), and —N(CH₃)₂. In some embodiments, R² is hydrogen or C₁₋₃ alkyl. In some embodiments. R² unsubstituted C₁₋₃ alkyl. In some embodiments, R² is C₁₋₃ alkyl substituted with a group selected from —OH, —NH₂, —NH(CH₃), and —N(CH₃)₂.

In some embodiments, R² is hydrogen. In some embodiments, R² is —OH. In some embodiments, R² is —CN. In some embodiments, R² is cyclopropyl.

In some embodiments, R² is halo. In some embodiments, R² is Cl, F, or Br. In some embodiments, R² is Cl. In some embodiments, R² is F. In some embodiments, R² is Br.

In some embodiments, R² is C₁₋₃ alkyl. In some embodiments, R² is methyl. In some embodiments, R² is ethyl. In some embodiments, R² is i-propyl. In some embodiments, R² is n-propyl.

In some embodiments, R² is C₁₋₃ haloalkyl. In some embodiments, R² is CF₃. In some embodiments, R² is CHF₂.

In some embodiments, R² is C₁₋₃ alkoxy. In some embodiments, R² is —CH₂OCH₃. In some embodiments, R₂ is —CH₂OCH₃. In some embodiments, R₂ is —CH₂CH₂OCH₃.

In some embodiments, R² is selected from those depicted in Table 1, below.

As defined generally above, R³ is hydrogen, cyclopropyl, or C₁₋₃ alkyl optionally substituted with a group selected from —OH, OMe, —NH₂, —NH(CH₃), and —N(CH₃)₂.

In some embodiments, R³ is cyclopropyl, or C₁₋₃ alkyl optionally substituted with a group selected from —OH, —OMe, —NH₂, —NH(CH₃), and —N(CH₃)₂. In some embodiments, R³ is hydrogen, cyclopropyl, or C₁₋₃ alkyl. In some embodiments, R³ is hydrogen or C₁₋₃ alkyl optionally substituted with a group selected from —OH, —OMe, —NH₂, —NH(CH₃), and —N(CH₃)₂. In some embodiments, R³ is hydrogen, cyclopropyl, or C₁₋₃ alkyl substituted with a group selected from —OH, —OMe, —NH₂, —NH(CH₃), and —N(CH₃)₂. In some embodiments, R³ is substituted C₁₋₃ alkyl as defined above and herein, wherein the substitution occurs on the terminal carbon atom.

In some embodiments, R³ is hydrogen or C₁₋₃ alkyl optionally substituted with a group selected from —OH and OMe. In some embodiments, R³ is hydrogen or C₁₋₃ alkyl.

In some embodiments, R³ is hydrogen. In some embodiments, R³ is cyclopropyl. In some embodiments, R³ is C₁₋₃ alkyl. In some embodiments, R³ is methyl. In some embodiments, R³ is ethyl. In some embodiments, R³ is i-propyl. In some embodiments, R³ is n-propyl.

In some embodiments, R³ is C₁₋₃ alkyl substituted with a group selected from —OH, OMe, —NH₂, —NH(CH₃), and —N(CH₃)₂. In some embodiments, R³ is C₁₋₃ alkyl substituted with a group selected from —OH or OMe. In some embodiments, R³ is C₁₋₃ alkyl substituted with a group selected from —OMe, —NH₂, —NH(CH₃), or —N(CH₃)₂. In some embodiments, R³ is C₁₋₃ alkyl substituted with a group selected from —NH₂, —NH(CH₃), or —N(CH₃)₂. In some embodiments, R³ is —CH₂OCH₃. In some embodiments, R³ is —CH₂OCH₃. In some embodiments, R³ is —CH₂CH₂OCH₃.

In some embodiments, R³ is selected from those depicted in Table 1, below.

As defined generally above, R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a 3-8 membered saturated or partially unsaturated monocyclic carbocyclic ring, wherein R⁴ is substituted with p instances of R⁵. In some embodiments, R⁴ is a 7-12 membered saturated or partially unsaturated bicyclic carbocyclic ring, wherein R⁴ is substituted with p instances of R⁵. In some embodiments, R⁴ is a phenyl, wherein R⁴ is substituted with p instances of R⁵. In some embodiments, R⁴ is an 8-10 membered bicyclic aromatic carbocyclic ring wherein R⁴ is substituted with p instances of R⁵. In some embodiments, R⁴ is a 3-8 membered saturated or partially unsaturated monocyclic heterocyclic ring having 1-4 heteroatoms independently selected from nitrogen, oxygen, and sulfur, wherein R⁴ is substituted with p instances of R⁵. In some embodiments, R⁴ is a 7-12 membered saturated or partially unsaturated bicyclic heterocyclic ring having 1-4 heteroatoms independently selected from nitrogen, oxygen, and sulfur, wherein R⁴ is substituted with p instances of R⁵. In some embodiments, R⁴ is a 5-6 membered monocyclic heteroaromatic ring having 1-4 heteroatoms independently selected from nitrogen, oxygen, and sulfur, wherein R⁴ is substituted with p instances of R⁵. In some embodiments, R⁴ is and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a 3-8 membered saturated or partially unsaturated monocyclic carbocyclic ring. In some embodiments, R⁴ is a 7-12 membered saturated or partially unsaturated bicyclic carbocyclic ring. In some embodiments, R⁴ is a phenyl. In some embodiments, R⁴ is an 8-10 membered bicyclic aromatic carbocyclic ring wherein R⁴ is substituted with p instances of R⁵. In some embodiments, R⁴ is a 3-8 membered saturated or partially unsaturated monocyclic heterocyclic ring having 1-4 heteroatoms independently selected from nitrogen, oxygen, and sulfur. In some embodiments, R⁴ is a 7-12 membered saturated or partially unsaturated bicyclic heterocyclic ring having 1-4 heteroatoms independently selected from nitrogen, oxygen, and sulfur. In some embodiments, R⁴ is a 5-6 membered monocyclic heteroaromatic ring having 1-4 heteroatoms independently selected from nitrogen, oxygen, and sulfur. In some embodiments, R⁴ is and an 8-10 membered bicyclic heteroaromatic ring having 1-5 heteroatoms independently selected from nitrogen, oxygen, and sulfur.

In some embodiments, R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, wherein R⁴ is substituted with p instances of R⁵.

In some embodiments, R⁴ is a cyclic group selected a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 7-12 membered saturated or partially unsaturated bicyclic carbocyclic ring, an 8-10 membered bicyclic aromatic carbocyclic ring, a 7-12 membered saturated or partially unsaturated bicyclic heterocyclic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 3-8 membered saturated or partially unsaturated monocyclic carbocyclic ring, phenyl, an 8-10 membered bicyclic aromatic carbocyclic ring, a 5-6 membered monocyclic heteroaromatic ring having 1-4 heteroatoms independently selected from nitrogen, oxygen, and sulfur, wherein R⁴ is substituted with p instances of R⁵.

In some embodiments, R⁴ is a cyclic group selected from a 3-6 membered saturated or partially unsaturated monocyclic carbocyclic ring, a 8-10 membered saturated or partially unsaturated bicyclic carbocyclic ring, phenyl, an 8-10 membered bicyclic aromatic carbocyclic ring, a 3-6 membered saturated or partially unsaturated monocyclic heterocyclic ring having 1-4 heteroatoms independently selected from nitrogen, oxygen, and sulfur, a 8-10 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 3-6 membered saturated or partially unsaturated monocyclic carbocyclic ring, a 8-10 membered saturated or partially unsaturated bicyclic carbocyclic ring, phenyl, an 8-10 membered bicyclic aromatic carbocyclic ring, a 3-6 membered saturated or partially unsaturated monocyclic heterocyclic ring having 2-4 heteroatoms independently selected from nitrogen, oxygen, and sulfur, a 8-10 membered saturated or partially unsaturated bicyclic heterocyclic ring having 2-4 heteroatoms independently selected from nitrogen, oxygen, and sulfur, a 5-6 membered monocyclic heteroaromatic ring having 2-4 heteroatoms independently selected from nitrogen, oxygen, and sulfur, and an 8-10 membered bicyclic heteroaromatic ring having 2-5 heteroatoms independently selected from nitrogen, oxygen, and sulfur, wherein R⁴ is substituted with p instances of R⁵.

In some embodiments, R⁴ is selected from those depicted in Table 1, below.

As defined generally above, R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence halo. In certain embodiments, R⁵ represents independently for each occurrence —OR. In certain embodiments, R⁵ represents independently for each occurrence —SR. In certain embodiments, R⁵ represents independently for each occurrence —NR₂. In certain embodiments, R⁵ represents independently for each occurrence —S(O)₂R. In certain embodiments, R⁵ represents independently for each occurrence —S(O)₂NR₂. In certain embodiments, R⁵ represents independently for each occurrence —S(O)R. In certain embodiments, R⁵ represents independently for each occurrence —S(O)NR₂. In certain embodiments, R⁵ represents independently for each occurrence —C(O)R. In certain embodiments, R⁵ represents independently for each occurrence —C(O)CR₃. In certain embodiments, R⁵ represents independently for each occurrence —C(O)OR. In certain embodiments, R⁵ represents independently for each occurrence —C(O)NR₂. In certain embodiments, R⁵ represents independently for each occurrence —C(O)N(R)OR. In certain embodiments, R⁵ represents independently for each occurrence —OC(O)R. In certain embodiments, R⁵ represents independently for each occurrence —OC(O)NR₂. In certain embodiments, R⁵ represents independently for each occurrence —O(CR₂)₃NR₂. In certain embodiments, R⁵ represents independently for each occurrence —N(R)C(O)OR. In certain embodiments, R⁵ represents independently for each occurrence —N(R)C(O)R. In certain embodiments, R⁵ represents independently for each occurrence —N(R)C(O)NR₂. In certain embodiments, R⁵ represents independently for each occurrence —N(R)C(NR)NR₂. In certain embodiments, R⁵ represents independently for each occurrence-NR(CR₂)₂OR. In certain embodiments, R⁵ represents independently for each occurrence —N(R)S(O)₂NR₂. In certain embodiments, R⁵ represents independently for each occurrence —N(R)S(O)₂R. In certain embodiments, R⁵ represents independently for each occurrence —CH₂R. In certain embodiments, R⁵ represents independently for each occurrence —CHR₂. In certain embodiments, R⁵ represents independently for each occurrence —CR₃. In certain embodiments, R⁵ represents independently for each occurrence —CH₂NR₂. In certain embodiments, R⁵ represents independently for each occurrence —CH₂NHCH₂CH₂OR. In certain embodiments, R⁵ represents independently for each occurrence —CH₂CH₂C(O)OR. In certain embodiments, R⁵ represents independently for each occurrence —CH₂C(O)NR₂. In certain embodiments, R⁵ represents independently for each occurrence —CH₂CH₂R, In certain embodiments, R⁵ represents independently for each occurrence —CH₂CH₂OR. In certain embodiments, R⁵ represents independently for each occurrence —NHCH₂CH₂OR. In certain embodiments, R⁵ represents independently for each occurrence —NHC(O)R. In certain embodiments, R⁵ represents independently for each occurrence —CH₂OR. In certain embodiments, R⁵ represents independently for each occurrence —CH₂NHC(O)OCR₃. In certain embodiments, R⁵ represents independently for each occurrence C₁₋₃ alkyl group. In certain embodiments, R⁵ represents independently for each occurrence a C₁₋₃ haloalkyl group. In certain embodiments, R⁵ represents independently for each occurrence a C₁₋₃ heteroalkyl group having 1-2 heteroatoms independently selected from nitrogen, oxygen, and sulfur. In certain embodiments, R⁵ represents independently for each occurrence a 3-6 membered saturated or partially unsaturated monocyclic carbocyclic ring. In certain embodiments, R⁵ represents independently for each occurrence phenyl. In certain embodiments, R⁵ represents independently for each occurrence a 5-6 membered monocyclic heteroaromatic ring having 1-4 heteroatoms independently selected from nitrogen, oxygen, and sulfur. In certain embodiments, R⁵ represents independently for each occurrence a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence oxo, —OR, —OC(O)R, —OC(O)NR₂, or —O(CR₂)₃NR₂.

In certain embodiments, R⁵ represents independently for each occurrence —CN, —C(O)R, —C(O)CR₃, —C(O)OR, —C(O)NR₂, —C(O)N(R)OR, —CR₃, —CH₂NR₂, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In certain embodiments, R⁵ represents independently for each occurrence a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, or a C₁₋₃ haloalkyl group.

In certain embodiments, R⁵ represents independently for each occurrence halo, oxo, —CN, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence halo, oxo, a C₁₋₃ alkyl group, a C₁₋₃ haloalkyl group, or a C₁₋₃ heteroalkyl group having 1-2 heteroatoms independently selected from nitrogen, oxygen, and sulfur.

In certain embodiments, R⁵ is halo. In certain embodiments, R⁵ is —OR. In certain embodiments, R⁵ is —SR. In certain embodiments, R⁵ is —NR₂. In certain embodiments, R⁵ is —S(O)₂R. In certain embodiments, R⁵ is —S(O)₂NR₂. In certain embodiments, R⁵ is —S(O)R. In certain embodiments, R⁵ is —S(O)NR₂. In certain embodiments, R⁵ is —C(O)R. In certain embodiments, R⁵ is —C(O)CR₃. In certain embodiments, R⁵ is —C(O)OR. In certain embodiments, R⁵ is —C(O)NR₂. In certain embodiments, R⁵ is —C(O)N(R)OR. In certain embodiments, R⁵ is —OC(O)R. In certain embodiments, R⁵ is —OC(O)NR₂. In certain embodiments, R⁵ is —O(CR₂)₃NR₂. In certain embodiments, R⁵ is —N(R)C(O)OR. In certain embodiments, R⁵ is —N(R)C(O)R. In certain embodiments, R⁵ is —N(R)C(O)NR₂. In certain embodiments, R⁵ is —N(R)C(NR)NR₂. In certain embodiments, R⁵ is —NR(CR₂)₂OR. In certain embodiments, R⁵ is —N(R)S(O)₂NR₂. In certain embodiments, R⁵ is —N(R)S(O)₂R. In certain embodiments, R⁵ is —CH₂R. In certain embodiments, R⁵ is —CHR₂. In certain embodiments, R⁵ is —CR₃. In certain embodiments, R⁵ is —CH₂NR₂. In certain embodiments, R⁵ is —CH₂NHCH₂CH₂OR. In certain embodiments, R⁵ is —CH₂CH₂C(O)OR. In certain embodiments, R⁵ is —CH₂C(O)NR₂. In certain embodiments, R⁵ is —CH₂CH₂R, In certain embodiments, R⁵ is —CH₂CH₂OR. In certain embodiments, R⁵ is —NHCH₂CH₂OR. In certain embodiments, R⁵ is —NHC(O)R. In certain embodiments, R⁵ is —CH₂OR. In certain embodiments, R⁵ is —CH₂NHC(O)OCR₃. In certain embodiments, R⁵ isa C₁₋₃ alkyl group. In certain embodiments, R⁵ is a C₁₋₃ haloalkyl group. In certain embodiments, R⁵ is a C₁₋₃ heteroalkyl group having 1-2 heteroatoms independently selected from nitrogen, oxygen, and sulfur. In certain embodiments, R⁵ is a 3-6 membered saturated or partially unsaturated monocyclic carbocyclic ring. In certain embodiments, R⁵ is phenyl. In certain embodiments, R⁵ is a 5-6 membered monocyclic heteroaromatic ring having 1-4 heteroatoms independently selected from nitrogen, oxygen, and sulfur. In certain embodiments, R⁵ is a 3-6 membered saturated or partially unsaturated monocyclic heterocyclic ring having 1-4 heteroatoms independently selected from nitrogen, oxygen, and sulfur.

In certain embodiments, R⁵ is oxo, —OR, —OC(O)R, —OC(O)NR₂, or —O(CR₂)₃NR₂.

In certain embodiments, R⁵ is —CN, —C(O)R, —C(O)CR₃, —C(O)OR, —C(O)NR₂, —C(O)N(R)OR, —CR₃, —CH₂NR₂—, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ is N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In certain embodiments, R⁵ is a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ is halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂—, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, or a C₁₋₃ haloalkyl group.

In certain embodiments, R⁵ is halo, oxo, —CN, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ is halo, oxo, a C₁₋₃ alkyl group, a C₁₋₃ haloalkyl group, or a C₁₋₃ heteroalkyl group having 1-2 heteroatoms independently selected from nitrogen, oxygen, and sulfur.

In some embodiments, R⁵ is selected from those depicted in Table 1, below.

As defined generally above, Y represents independently for each occurrence —R, oxo, —OR, —NR₂, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y represents independently for each occurrence —R. In certain embodiments, Y represents independently for each occurrence —OR. In certain embodiments, Y represents independently for each occurrence —NR₂. In certain embodiments, Y represents independently for each occurrence —NHCH₂CH₂OR. In certain embodiments, two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y represents independently for each occurrence oxo, —OR, —NR₂, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y represents independently for each occurrence —R, —OR, —NR₂, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y represents independently for each occurrence —R, oxo, —NR₂, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y represents independently for each occurrence —R, oxo, —OR, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y represents independently for each occurrence —R, oxo, —OR, —NR₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y represents independently for each occurrence —R, oxo, —OR, —NR₂, —OCH₂CHF₂, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y represents independently for each occurrence —R, oxo, —OR, —NR₂, —OCH₂CHF₂, or —OCH₂CH₂OH; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y represents independently for each occurrence —R, oxo, —OR, —NR₂, —OCH₂CHF₂, —OCH₂CH₂OH, or —NHCH₂CH₂OR.

In certain embodiments, Y represents independently for each occurrence —OH, halogen, optionally substituted C₁₋₆ aliphatic, phenyl, oxo, —OR, —NR₂, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y represents independently for each occurrence —OH, halogen, methyl, ethyl, i-propyl, n-propyl, phenyl, oxo, —OCH₃, —NR₂, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y represents independently for each occurrence —OH, halogen, methyl, ethyl, i-propyl, n-propyl, phenyl; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y is —R. In certain embodiments, Y is —OR. In certain embodiments, Y is —NR₂. In certain embodiments, Y is —NHCH₂CH₂OR. In certain embodiments, Y is —OCH₂CHF₂. In certain embodiments, Y is —OCH₂CH₂OH. In certain embodiments, two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y is oxo, —OR, —NR₂, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y is —R, —OR, —NR₂, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y is —R, oxo, —NR₂, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y is —R, oxo, —OR, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y is —R, oxo, —OR, —NR₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y is —R, oxo, —OR, —NR₂, —OCH₂CHF₂, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y is —R, oxo, —OR, —NR₂, —OCH₂CHF₂, or —OCH₂CH₂OH; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y is —R, oxo, —OR, —NR₂, —OCH₂CHF₂, —OCH₂CH₂OH, or —NHCH₂CH₂OR.

In certain embodiments, Y is —OH, halogen, optionally substituted C₁₋₆ aliphatic, phenyl, oxo, —OR, —NR₂, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y is —OH, halogen, methyl, ethyl, i-propyl, n-propyl, phenyl, oxo, —OCH₃, —NR₂, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y is —OH, halogen, methyl, ethyl, i-propyl, n-propyl, phenyl; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur.

In certain embodiments, Y is methyl. In certain embodiments, Y is phenyl.

In some embodiments, Y is selected from those depicted in Table 1, below.

As defined generally above, L¹ is a covalent bond, —OCH₂—, —O—, —OCH₂CH₂—, —NHCH₂—, —NHCH₂CH₂—, or —NH—.

In certain embodiments, L¹ is a covalent bond. In certain embodiments, L¹ is —OCH₂—. In certain embodiments, L¹ is —O—. In certain embodiments, L¹ is —OCH₂CH₂—. In certain embodiments, L¹ is —NHCH₂—. In certain embodiments, L¹ is —NHCH₂CH₂—. In certain embodiments, L¹ is —NH—.

In certain embodiments, L¹ is —OCH₂—, —O—, —OCH₂CH₂—, —NHCH₂—, —NHCH₂CH₂—, or —NH—. In certain embodiments, L¹ is a covalent bond, —O—, —OCH₂CH₂—, —NHCH₂—, —NHCH₂CH₂—, or —NH—. In certain embodiments, L¹ is a covalent bond, —OCH₂—, —OCH₂CH₂—, —NHCH₂—, —NHCH₂CH₂—, or —NH—. In certain embodiments, L¹ is a covalent bond, —OCH₂—, —O—, —NHCH₂—, —NHCH₂CH₂—, or —NH—. In certain embodiments, L¹ is a covalent bond, —OCH₂—, —O—, —OCH₂CH₂—, —NHCH₂CH₂—, or —NH—. In certain embodiments, L¹ is a covalent bond, —OCH₂—, —O—, —OCH₂CH₂—, —NHCH₂—, or —NH—. In certain embodiments, L¹ is a covalent bond, —OCH₂—, —O—, —OCH₂CH₂—, —NHCH₂—, or —NHCH₂CH₂—.

In some embodiments, L¹ is selected from those depicted in Table 1, below.

As defined generally above, R represents independently for each occurrence hydrogen, hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence halo. In certain embodiments, R represents independently for each occurrence optionally substituted C₁₋₆ aliphatic. In certain embodiments, R represents independently for each occurrence optionally substituted phenyl. In certain embodiments, R represents independently for each occurrence optionally substituted 3-7 membered saturated or partially unsaturated monocyclic heterocyclic ring having 1-4 heteroatoms independently selected from nitrogen, oxygen, and sulfur. In certain embodiments, R represents independently for each occurrence optionally substituted 5-6 membered monocyclic heteroaryl ring having 1-4 heteroatoms independently selected from nitrogen, oxygen, and sulfur. In certain embodiments, R represents independently for each occurrence optionally substituted 3-7 membered saturated or partially unsaturated monocyclic carbocyclic ring.

In certain embodiments, R represents independently for each occurrence halo. In certain embodiments, R represents independently for each occurrence C₁₋₆ aliphatic. In certain embodiments, R represents independently for each occurrence phenyl. In certain embodiments, R represents independently for each occurrence 3-7 membered saturated or partially unsaturated monocyclic heterocyclic ring having 1-4 heteroatoms independently selected from nitrogen, oxygen, and sulfur. In certain embodiments, R represents independently for each occurrence 5-6 membered monocyclic heteroaryl ring having 1-4 heteroatoms independently selected from nitrogen, oxygen, and sulfur. In certain embodiments, R represents independently for each occurrence 3-7 membered saturated or partially unsaturated monocyclic carbocyclic ring.

In certain embodiments, R represents independently for each occurrence hydrogen, hydroxyl, halo, or a group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence hydrogen, hydroxyl, halo, or optionally substituted C₁₋₆ aliphatic.

In certain embodiments, R represents independently for each occurrence an optionally substituted group selected from phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence hydrogen, hydroxyl, halo, or an optionally substituted group selected from C₁₋₃ aliphatic, phenyl, a 3-6 membered saturated or partially unsaturated monocyclic heterocyclic ring having 1-3 heteroatoms independently selected from nitrogen, oxygen, and sulfur, a 5-6 membered monocyclic heteroaryl ring having 1-3 heteroatoms independently selected from nitrogen, oxygen, and sulfur, and a 3-6 membered saturated or partially unsaturated monocyclic carbocyclic ring.

In certain embodiments, R represents independently for each occurrence hydrogen, hydroxyl, Cl, F, —CH₂OH, —CH₂CH₂OH, methyl, ethyl, i-propyl, n-propyl, cyclopropyl, or phenyl.

In certain embodiments, R is halo. In certain embodiments, R is optionally substituted C₁₋₆ aliphatic. In certain embodiments, R is optionally substituted phenyl. In certain embodiments, R is optionally substituted 3-7 membered saturated or partially unsaturated monocyclic heterocyclic ring having 1-4 heteroatoms independently selected from nitrogen, oxygen, and sulfur. In certain embodiments, R is optionally substituted 5-6 membered monocyclic heteroaryl ring having 1-4 heteroatoms independently selected from nitrogen, oxygen, and sulfur. In certain embodiments, R is optionally substituted 3-7 membered saturated or partially unsaturated monocyclic carbocyclic ring.

In certain embodiments, R is halo. In certain embodiments, R is C₁₋₆ aliphatic. In certain embodiments, R is phenyl. In certain embodiments, R is 3-7 membered saturated or partially unsaturated monocyclic heterocyclic ring having 1-4 heteroatoms independently selected from nitrogen, oxygen, and sulfur. In certain embodiments, R is 5-6 membered monocyclic heteroaryl ring having 1-4 heteroatoms independently selected from nitrogen, oxygen, and sulfur. In certain embodiments, R is 3-7 membered saturated or partially unsaturated monocyclic carbocyclic ring.

In certain embodiments, R is hydrogen, hydroxyl, halo, or a group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R is hydrogen, hydroxyl, halo, or optionally substituted C₁₋₆ aliphatic.

In certain embodiments, R is an optionally substituted group selected from phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R is hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R is hydrogen, hydroxyl, halo, or an optionally substituted group selected from C₁a aliphatic, phenyl, a 3-6 membered saturated or partially unsaturated monocyclic heterocyclic ring having 1-3 heteroatoms independently selected from nitrogen, oxygen, and sulfur, a 5-6 membered monocyclic heteroaryl ring having 1-3 heteroatoms independently selected from nitrogen, oxygen, and sulfur, and a 3-6 membered saturated or partially unsaturated monocyclic carbocyclic ring.

In certain embodiments, R is hydrogen, hydroxyl, Cl, F, —CH₂OH, —CH₂CH₂OH, methyl, ethyl, i-propyl, n-propyl, cyclopropyl, or phenyl.

In some embodiments, R is selected from those depicted in Table 1, below.

As defined generally above, n is 0, 1, 2, or 3. In certain embodiments, n is 1, 2, or 3. In certain embodiments, n is 0, 2, or 3. In certain embodiments, n is 0, 1, or 3. In certain embodiments, n is 0, 1, or 2. In certain embodiments, n is 2 or 3. In certain embodiments, n is 0 or 1. In certain embodiments, n is 0 or 3. In certain embodiments, n is 0 or 2. In certain embodiments, n is 1 or 2. In certain embodiments, n is 1 or 3. In certain embodiments, n is 0. In certain embodiments, n is 1. In certain embodiments, n is 2. In certain embodiments, n is 3.

As defined generally above, p is 0, 1, 2, 3 or 4. In certain embodiments, p is 1, 2, 3 or 4. In certain embodiments, p is 0, 2, 3 or 4. In certain embodiments, p is 0, 1, 3 or 4. In certain embodiments, p is 0, 1, 2, or 4. In certain embodiments, p is 0, 1, 2, or 3. In certain embodiments, p is 2, 3 or 4. In certain embodiments, p is 0, 3 or 4. In certain embodiments, p is 0, 1, or 4. In certain embodiments, p is 0, 1, or 2. In certain embodiments, p is 1, 3 or 4. In certain embodiments, p is 1, 2, or 4. In certain embodiments, p is 1, 2, or 3. In certain embodiments, p is 0, 2 or 4. In certain embodiments, p is 0, 2 or 3. In certain embodiments, p is 0, 1 or 3. In certain embodiments, p is 3 or 4. In certain embodiments, p is 0 or 4. In certain embodiments, p is 0 or 1. In certain embodiments, p is 1 or 4. In certain embodiments, p is 1 or 2. In certain embodiments, p is 0 or 2. In certain embodiments, p is 0 or 3. In certain embodiments, p is 1 or 3. In certain embodiments, p is 2 or 3. In certain embodiments, p is 2 or 4. In certain embodiments, p is 0. In certain embodiments, p is 1. In certain embodiments, p is 2. In certain embodiments, p is 3. In certain embodiments, p is 4.

One aspect of the invention provides a compound represented by Formula III:

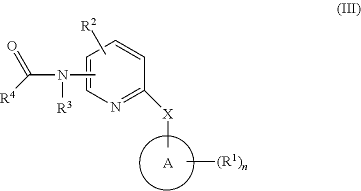

or a pharmaceutically acceptable salt thereof; wherein:
Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur, a 4-8 membered saturated or partially unsaturated monocyclic heterocyclic ring containing 1-4 heteroatoms independently selected from nitrogen, oxygen, and sulfur, a 5-6 membered monocyclic heteroaromatic ring having 1-4 heteroatoms independently selected from nitrogen, oxygen, and sulfur, a 6-11 membered saturated or partially unsaturated bicyclic carbocyclic ring, a 3-8 membered saturated or partially unsaturated monocyclic carbocyclic ring, an 8-10 membered bicyclic aromatic carbocyclic ring, or phenyl;
R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, C₁₋₄ haloaliphatic, or a cyclic group selected from a 3-8 membered saturated monocyclic carbocyclic ring or a 3-8 membered saturated monocyclic heterocyclic ring having 1-4 heteroatoms independently selected from nitrogen, oxygen, and sulfur;
R² is hydrogen, halo, —OH, —CN, cyclopropyl. C₁₋₃ alkyl optionally substituted with a group selected from —OH, —NH₂, —NH(CH₃), and —N(CH₃)₂, C₁₋₃ haloalkyl, or C₁₋₃ alkoxy;
R³ is hydrogen, cyclopropyl, or C₁₋₃ alkyl optionally substituted with a group selected from —OH, —OMe, —NH₂, —NH(CH₃), and —N(CH₃)₂;
R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵;
R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur;
X is —O— or —NH—;
R represents independently for each occurrence hydrogen, hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring;
n is 0, 1, 2, or 3; and
p is 0, 1, 2, 3 or 4.

The definitions of variables in Formula III above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula III.

As defined generally above, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur; a 5-6 membered monocyclic heteroaromatic ring having 1-4 heteroatoms independently selected from nitrogen, oxygen, and sulfur: a 6-11 membered saturated or partially unsaturated bicyclic carbocyclic ring; a 3-8 membered saturated or partially unsaturated monocyclic carbocyclic ring; an 8-10 membered bicyclic aromatic carbocyclic ring; or phenyl.

In some embodiments, Ring A is a 6-14 membered saturated or partially unsaturated bicyclic heterocyclic ring containing 1-4 heteroatoms independently selected from nitrogen, oxygen, and sulfur. In some embodiments, Ring A is an 8-10 membered bicyclic heteroaromatic ring having 1-4 heteroatoms independently selected from nitrogen, oxygen, and sulfur. In some embodiments. Ring A is a 4-8 membered saturated or partially unsaturated monocyclic heterocyclic ring containing 1-4 heteroatoms independently selected from nitrogen, oxygen, and sulfur. In some embodiments, Ring A is a 5-6 membered monocyclic heteroaromatic ring having 1-4 heteroatoms independently selected from nitrogen, oxygen, and sulfur. In some embodiments, Ring A is a 6-11 membered saturated or partially unsaturated bicyclic carbocyclic ring; a 3-8 membered saturated or partially unsaturated monocyclic carbocyclic ring. In some embodiments, Ring A is an 8-10 membered bicyclic aromatic carbocyclic ring. In some embodiments, Ring A is phenyl.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur or a 5-6 membered monocyclic heteroaromatic ring having 1-4 heteroatoms independently selected from nitrogen, oxygen, and sulfur.

In some embodiments, Ring A is a 6-14 membered saturated or partially unsaturated bicyclic heterocyclic ring containing 1-4 heteroatoms independently selected from nitrogen, oxygen, and sulfur; or an 8-10 membered bicyclic heteroaromatic ring having 1-4 heteroatoms independently selected from nitrogen, oxygen, and sulfur.

In some embodiments, Ring A is a 4-8 membered saturated or partially unsaturated monocyclic heterocyclic ring containing 1-4 heteroatoms independently selected from nitrogen, oxygen, and sulfur or a 5-6 membered monocyclic heteroaromatic ring having 1-4 heteroatoms independently selected from nitrogen, oxygen, and sulfur.

In some embodiments, Ring A is a 6-11 membered saturated or partially unsaturated bicyclic carbocyclic ring; a 3-8 membered saturated or partially unsaturated monocyclic carbocyclic ring; an 8-10 membered bicyclic aromatic carbocyclic ring; or phenyl.

In some embodiments, Ring A is a 3-8 membered saturated or partially unsaturated monocyclic carbocyclic ring or phenyl.

In some embodiments, Ring A is a 6-11 membered saturated or partially unsaturated bicyclic carbocyclic ring; or an 8-10 membered bicyclic aromatic carbocyclic ring.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur; a 5-6 membered monocyclic heteroaromatic ring having 1-4 heteroatoms independently selected from nitrogen, oxygen, and sulfur; or a 6-11 membered saturated or partially unsaturated bicyclic carbocyclic ring; a 3-8 membered saturated or partially unsaturated monocyclic carbocyclic ring.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur; a 5-6 membered monocyclic heteroaromatic ring having 1-4 heteroatoms independently selected from oxygen, and sulfur; a 6-11 membered saturated or partially unsaturated bicyclic carbocyclic ring; a 3-8 membered saturated or partially unsaturated monocyclic carbocyclic ring; or phenyl.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur; a 6-11 membered saturated or partially unsaturated bicyclic carbocyclic ring; a 3-8 membered saturated or partially unsaturated monocyclic carbocyclic ring; or phenyl.

In some embodiments, Ring A is selected from those depicted in Table 1, below.

As defined generally above, R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments. R¹ represents independently for each occurrence halo. In some embodiments, R¹ represents independently for each occurrence —OR. In some embodiments, R¹ represents independently for each occurrence —SR. In some embodiments, R¹ represents independently for each occurrence —NR₂. In some embodiments, R¹ represents independently for each occurrence —S(O)₂R. In some embodiments, R¹ represents independently for each occurrence —S(O)₂NR₂. In some embodiments, R¹ represents independently for each occurrence —S(O)R. In some embodiments, R¹ represents independently for each occurrence —S(O)NR₂. In some embodiments, R¹ represents independently for each occurrence —C(O)R. In some embodiments, R¹ represents independently for each occurrence —C(O)CR₃. In some embodiments, R¹ represents independently for each occurrence —C(O)OR. In some embodiments, R¹ represents independently for each occurrence —C(O)NR₂. In some embodiments, R¹ represents independently for each occurrence —C(O)N(R)OR. In some embodiments, R¹ represents independently for each occurrence —OC(O)R. In some embodiments. R¹ represents independently for each occurrence —OC(O)NR₂. In some embodiments, R¹ represents independently for each occurrence —O(CR₂)₃NR₂. In some embodiments, R¹ represents independently for each occurrence —N(R)C(O)OR. In some embodiments, R¹ represents independently for each occurrence —N(R)C(O)R. In some embodiments, R¹ represents independently for each occurrence —N(R)C(O)NR₂. In some embodiments, R¹ represents independently for each occurrence —N(R)C(NR)NR₂. In some embodiments, R¹ represents independently for each occurrence —NR(CR₂)₂OR. In some embodiments, R¹ represents independently for each occurrence —N(R)S(O)₂NR₂. In some embodiments, R¹ represents independently for each occurrence —N(R)S(O)₂R. In some embodiments, R¹ represents independently for each occurrence —CH₂R. In some embodiments, R¹ represents independently for each occurrence —CHR₂. In some embodiments, R¹ represents independently for each occurrence —CR₃. In some embodiments, R¹ represents independently for each occurrence —CH₂NR₂. In some embodiments, R¹ represents independently for each occurrence —CH₂NHCH₂CH₂OR. In some embodiments, R¹ represents independently for each occurrence —CH₂CH₂C(O)OR. In some embodiments, R¹ represents independently for each occurrence —CH₂C(O)NR₂. In some embodiments, R¹ represents independently for each occurrence —CH₂CH₂R. In some embodiments, R¹ represents independently for each occurrence —CH₂CH₂OR. In some embodiments, R¹ represents independently for each occurrence —NHCH₂CH₂OR. In some embodiments, R¹ represents independently for each occurrence —NHC(O)R. In some embodiments, R¹ represents independently for each occurrence —CH₂OR. In some embodiments, R¹ represents independently for each occurrence —CH₂C(O)NH₂. In some embodiments, R¹ represents independently for each occurrence —CH₂NHC(O)OCR₃. In some embodiments, R¹ represents independently for each occurrence —OCR. In some embodiments, R¹ represents independently for each occurrence C₁₋₄ aliphatic. In some embodiments. R¹ represents independently for each occurrence C₁₋₄ haloaliphatic. In some embodiments, R¹ represents independently for each occurrence a 3-8 membered saturated monocyclic carbocyclic ring. In some embodiments, R¹ represents independently for each occurrence a 3-8 membered saturated monocyclic heterocyclic ring having 1-4 heteroatoms independently selected from nitrogen, oxygen, and sulfur.

In some embodiments, R¹ represents independently for each occurrence —CN, —C(O)R, —C(O)CR₃, —C(O)OR, —C(O)NR₂, —C(O)N(R)OR, —CR₃, —CH₂NR₂—, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ represents independently for each occurrence halo, oxo, —OR, —SR, —NR₂, —S(O)₂R, —S(O)₂NR₂, —S(O)R, —S(O)NR₂, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, —NHC(O)R, or —OCR.

In some embodiments, R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, or C₁₋₄ haloaliphatic.

In some embodiments, R¹ represents independently for each occurrence halo, oxo, —CN, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ represents independently for each occurrence oxo, —OR, —OC(O)R, —OC(O)NR₂, —O(CR₂)₃NR₂, or —OCR.

In some embodiments, R¹ represents independently for each occurrence —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In some embodiments, R¹ represents independently for each occurrence halo, oxo, —OR, —C(O)R, —C(O)CR₃, —C(O)OR, —N(R)C(O)OR, —N(R)C(O)R, —N(R)C(O)NR₂, —CR₃, —CH₂OR, —CH₂C(O)NH₂, or C₁₋₄ aliphatic.

In some embodiments, R¹ represents independently for each occurrence oxo or C₁₋₄ aliphatic.

In some embodiments, R¹ represents independently for each occurrence —CH₃, —CH₂C(O)NH₂, —CH₂OCH₃, —CH₂CH₂OCH₃, —NHAc, —OH, —C(O)CH₂OH, —C(O)CH₃, —CH₂NHCH₂CH₂OH, —CH₂C(O)NH₂, —OCH₃, CH₂OCH₃, —C(O)OCH₃, —C(O)OH, —NHCH₂CH₂OH, —CH₂CH₂C(O)OCH₃, —NHS(O)₂CH₃, —C(O)NH₂, —S(O)₂CH₃, -Bn, —OC(CH₃)₂, —NHCH₂CH₂OCH₃, —F, —Cl, —Br, (O), —CH₂NH₂, —NH₂, —NHC(O)CH₃, —C(O)OCH₂CH₃, —S(O)₂NHCH₂CH₂OH, —CH₂CH₃, —N(CH₃)₂, —CH(CH₃)₂, —CF₃, —CN, —S(O)₂CH₃, —CH₂OH, —OCH₃, —CH₂N(CH₃)₂, —O(CH₂)₃N(CH₃)₂, —C(O)NH₂, -cyclohexyl, —OCF₃, —S(O)₂NH₂, —CHF₂, —CH₂NHC(O)OC(CH₃)₃, —C(CH₃)₂(OH), —CH(OH)CH₃, —O(CH₂)₂NH₂,

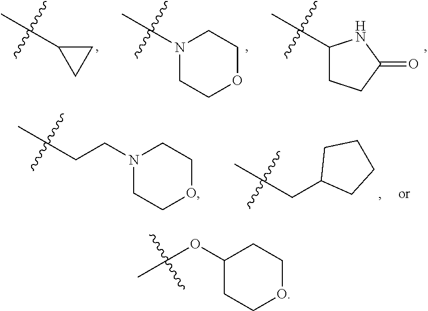

In some embodiments, R¹ represents independently for each occurrence —CH₃, —NHAc, —OH, —C(O)CH₂OH, —C(O)CH₃, —C(O)OCH₃, —C(O)OH, —C(O)NH₂, (O), —CH₂NH₂, —NH₂, —NHC(O)CH₃, —CH₂CH₃, —CH₂OH, —OCH₃, or

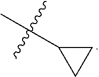 [SMILES: *C1CC1]

In some embodiments, R¹ represents independently for each occurrence —CH₃, —NHAc, —OH, (O), —OCH₃, or

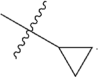 [SMILES: *C1CC1]

In some embodiments, R¹ represents independently for each occurrence —CH₃ or (O).

In some embodiments, R¹ is halo. In some embodiments, R¹ is —OR. In some embodiments, R¹ is —SR. In some embodiments, R¹ is —NR₂. In some embodiments, R¹ is —S(O)₂R. In some embodiments, R¹ is —S(O)₂NR₂. In some embodiments, R¹ is —S(O)R. In some embodiments, R¹ is —S(O)NR₂. In some embodiments, R¹ is —C(O)R. In some embodiments, R¹ is —C(O)CR₃. In some embodiments, R¹ is —C(O)OR. In some embodiments. R¹ is —C(O)NR₂. In some embodiments, R¹ is —C(O)N(R)OR. In some embodiments, R¹ is —OC(O)R. In some embodiments, R¹ is —OC(O)NR₂. In some embodiments, R¹ is —O(CR₂)₃NR₂. In some embodiments, R¹ is —N(R)C(O)OR. In some embodiments, R¹ is —N(R)C(O)R. In some embodiments, R¹ is —N(R)C(O)NR₂. In some embodiments, R¹ is —N(R)C(NR)NR₂. In some embodiments, R¹ is —NR(CR₂)₂OR. In some embodiments, R¹ is —N(R)S(O)₂NR₂. In some embodiments, R¹ is —N(R)S(O)₂R. In some embodiments, R¹ is —CR₃. In some embodiments, R¹ is —CH₂NR₂. In some embodiments, R¹ is —CH₂NHCH₂CH₂OR. In some embodiments, R¹ is —CH₂CH₂C(O)OR. In some embodiments. R¹ is —CH₂C(O)NR₂. In some embodiments, R¹ is —CH₂CH₂R. In some embodiments. R¹ is —CH₂CH₂OR. In some embodiments, R¹ is —NHCH₂CH₂OR. In some embodiments, R¹ is —NHC(O)R. In some embodiments, R¹ is —CH₂OR. In some embodiments, R¹ is —CH₂C(O)NH₂. In some embodiments, R¹ is —CH₂NHC(O)OCR₃. In some embodiments, R¹ is —OCR. In some embodiments, R¹ is C₁₋₄ aliphatic. In some embodiments. R¹ is C₁₋₄ haloaliphatic. In some embodiments, R¹ is a 3-8 membered saturated monocyclic carbocyclic ring. In some embodiments, R¹ is a 3-8 membered saturated monocyclic heterocyclic ring having 1-4 heteroatoms independently selected from nitrogen, oxygen, and sulfur.

In some embodiments, R¹ is —CN, —C(O)R, —C(O)CR₃, —C(O)OR, —C(O)NR₂, —C(O)N(R)OR, —CR₃, —CH₂NR₂, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃. C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ is halo, oxo, —OR, —SR, —NR₂, —S(O)₂R, —S(O)₂NR₂, —S(O)R, —S(O)NR₂, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, —NHC(O)R, or —OCR.

In some embodiments, R¹ is halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, or C₁₋₄ haloaliphatic.

In some embodiments, R¹ is halo, oxo, —CN, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ is oxo, —OR, —OC(O)R, —OC(O)NR₂, —O(CR₂)₃NR₂, or —OCR.

In some embodiments, R¹ is —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In some embodiments, R¹ is halo, oxo, —OR, —C(O)R, —C(O)CR₃, —C(O)OR, —N(R)C(O)OR, —N(R)C(O)R, —N(R)C(O)NR₂, —CR₃, —CH₂OR, —CH₂C(O)NH₂, or C₁₋₄ aliphatic.

In some embodiments, R¹ is oxo or C₁₋₄ aliphatic.

In some embodiments, R¹ is —CH₃, —CH₂C(O)NH₂, —CH₂OCH₃, —CH₂CH₂OCH₃, —NHAc, —OH, —C(O)CH₂OH, —C(O)CH₃, —CH₂NHCH₂CH₂OH, —CH₂C(O)NH₂, —OCH₃, CH₂OCH₃, —C(O)OCH₃, —C(O)OH, —NHCH₂CH₂OH, —CH₂CH₂C(O)OCH₃, —NHS(O)₂CH₃, —C(O)NH₂, —S(O)₂CH₃, -Bn, —OC(CH₃)₂, —NHCH₂CH₂OCH₃, —F, —Cl, —Br, (O), —CH₂NH₂, —NH₂, —NHC(O)CH₃, —C(O)OCH₂CH₃, —S(O)₂NHCH₂CH₂OH, —CH₂CH₃, —N(CH₃)₂, —CH(CH₃)₂, —CF₃, —CN, —S(O)₂CH₃, —CH₂OH, —OCH₃, —CH₂N(CH₃)₂, —O(CH₂)₃N(CH₃)₂, —C(O)NH₂, -cyclohexyl, —OCF₃, —S(O)₂NH₂, —CHF₂, —CH₂NHC(O)OC(CH₃)₃, —C(CH₃)₂(OH), —CH(OH)CH₃, —O(CH₂)₂NH₂,

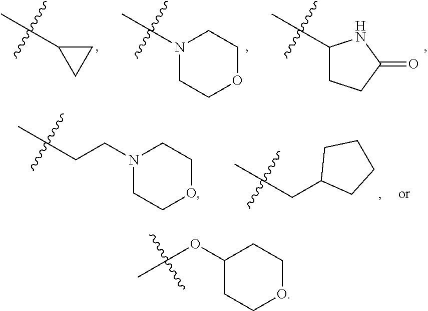

In some embodiments, R¹ is —CH₃, —NHAc, —OH, —C(O)CH₂OH, —C(O)CH₃, —C(O)OCH₃, —C(O)OH, —C(O)NH₂, (O), —CH₂NH₂, —NH₂, —NHC(O)CH₃, —CH₂CH₃, —CH₂OH, —OCH₃, or

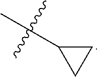 [SMILES: *C1CC1]

In some embodiments, R¹ is —CH₃, —NHAc, —OH, (O), —OCH₃, or

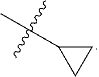 [SMILES: *C1CC1]

In some embodiments, R¹ is —CH₃ or (O).

In some embodiments, R¹ is selected from those depicted in Table 1, below.

As defined generally above, R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy.

In some embodiments, R² is halo, —OH, —CN, cyclopropyl. C₁₋₃ alkyl optionally substituted with a group selected from —OH, —NH₂, —NH(CH₃), and —N(CH₃)₂, C₁₋₃ haloalkyl, or C₁₋₃ alkoxy. In some embodiments, R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy. In some embodiments, R² is hydrogen, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy. In some embodiments, R² is hydrogen, halo, —CN, cyclopropyl, C₁₋₃ alkyl, C₁₋₃ haloalkyl, or C₁₋₃ alkoxy. In some embodiments, R² is hydrogen, halo, —OH, cyclopropyl, C₁₋₃ alkyl, C₁₋₃ haloalkyl, or C₁₋₃ alkoxy. In some embodiments. R² is hydrogen, halo, —OH, —CN, C₁₋₃ alkyl, C₁₋₃ haloalkyl, or C₁₋₃ alkoxy. In some embodiments. R² is hydrogen, halo, —OH, —CN, cyclopropyl, C₁₋₃ haloalkyl, or C₁₋₃ alkoxy. In some embodiments, R² is hydrogen, halo, —OH, —CN, cyclopropyl, C₁₋₃ alkyl, or C₁₋₃ alkoxy. In some embodiments, R² is hydrogen, halo, —OH, —CN, cyclopropyl, C₁₋₃ alkyl, or C₁₋₃ haloalkyl.

In some embodiments, R² is hydrogen, cyclopropyl, C₁₋₃ alkyl optionally substituted with a group selected from —OH, —NH₂, —NH(CH₃), and —N(CH₃)₂, or C₁₋₃ alkoxy. In some embodiments, R² is hydrogen or C₁₋₃ alkyl optionally substituted with a group selected from —OH, —NH₂, —NH(CH₃), and —N(CH₃)₂. In some embodiments, R² is hydrogen or C₁₋₃ alkyl. In some embodiments, R² unsubstituted C₁₋₃ alkyl. In some embodiments, R² is C₁₋₃ alkyl substituted with a group selected from —OH, —NH₂, —NH(CH₃), and —N(CH₃)₂.

In some embodiments, R² is hydrogen. In some embodiments, R² is —OH. In some embodiments, R² is —CN. In some embodiments, R² is cyclopropyl.

In some embodiments, R² is halo. In some embodiments, R² is Cl, F, or Br. In some embodiments, R² is Cl. In some embodiments, R² is F. In some embodiments, R² is Br.

In some embodiments, R² is C₁₋₃ alkyl. In some embodiments, R² is methyl. In some embodiments, R² is ethyl. In some embodiments. R² is i-propyl. In some embodiments, R² is n-propyl.

In some embodiments, R² is C₁₋₃ haloalkyl. In some embodiments, R² is CF₃. In some embodiments, R² is CHF₂.

In some embodiments, R² is C₁₋₃ alkoxy. In some embodiments, R² is —CH₂OCH₃. In some embodiments, R₂ is —CH₂OCH₃. In some embodiments, R₂ is —CH₂CH₂OCH₃.

In some embodiments, R² is selected from those depicted in Table 1, below.

As defined generally above, R³ is hydrogen, cyclopropyl, or C₁₋₃ alkyl optionally substituted with a group selected from —OH, OMe, —NH₂, —NH(CH₃), and —N(CH₃)₂.

In some embodiments, R³ is cyclopropyl, or C₁₋₃ alkyl optionally substituted with a group selected from —OH, —OMe, —NH₂, —NH(CH₃), and —N(CH₃)₂. In some embodiments, R³ is hydrogen, cyclopropyl, or C₁₋₃ alkyl. In some embodiments, R³ is hydrogen or C₁₋₃ alkyl optionally substituted with a group selected from —OH, —OMe, —NH₂, —NH(CH₃), and —N(CH₃)₂. In some embodiments, R³ is hydrogen, cyclopropyl, or C₁₋₃ alkyl substituted with a group selected from —OH, —OMe, —NH₂, —NH(CH₃), and —N(CH₃)₂. In some embodiments, R³ is substituted C₁₋₃ alkyl as defined above and herein, wherein the substitution occurs on the terminal carbon atom.

In some embodiments, R³ is hydrogen or C₁₋₃ alkyl optionally substituted with a group selected from —OH and OMe. In some embodiments. R³ is hydrogen or C₁₋₃ alkyl.

In some embodiments, R³ is hydrogen. In some embodiments. R³ is cyclopropyl. In some embodiments, R³ is C₁₋₃ alkyl. In some embodiments, R³ is methyl. In some embodiments, R³ is ethyl. In some embodiments, R³ is i-propyl. In some embodiments, R³ is n-propyl.

In some embodiments, R³ is C₁₋₃ alkyl substituted with a group selected from —OH, OMe, —NH₂, —NH(CH₃), and —N(CH₃)₂. In some embodiments, R³ is C₁₋₃ alkyl substituted with a group selected from —OH or OMe. In some embodiments, R³ is C₁₋₃ alkyl substituted with a group selected from —OMe, —NH₂, —NH(CH₃), or —N(CH₃)₂. In some embodiments, R³ is C₁₋₃ alkyl substituted with a group selected from —NH₂, —NH(CH₃), or —N(CH₃)₂. In some embodiments, R³ is —CH₂OCH₃. In some embodiments, R³ is —CH₂OCH₃. In some embodiments, R³ is —CH₂CH₂OCH₃.

In some embodiments, R³ is selected from those depicted in Table 1, below.

As defined generally above, R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a 3-8 membered saturated or partially unsaturated monocyclic carbocyclic ring, wherein R⁴ is substituted with p instances of R⁵. In some embodiments, R⁴ is a 7-12 membered saturated or partially unsaturated bicyclic carbocyclic ring, wherein R⁴ is substituted with p instances of R⁵. In some embodiments, R⁴ is a phenyl, wherein R⁴ is substituted with p instances of R⁵. In some embodiments, R⁴ is an 8-10 membered bicyclic aromatic carbocyclic ring wherein R⁴ is substituted with p instances of R⁵. In some embodiments, R⁴ is a 3-8 membered saturated or partially unsaturated monocyclic heterocyclic ring having 1-4 heteroatoms independently selected from nitrogen, oxygen, and sulfur, wherein R⁴ is substituted with p instances of R⁵. In some embodiments, R⁴ is a 7-12 membered saturated or partially unsaturated bicyclic heterocyclic ring having 1-4 heteroatoms independently selected from nitrogen, oxygen, and sulfur, wherein R⁴ is substituted with p instances of R⁵. In some embodiments, R⁴ is a 5-6 membered monocyclic heteroaromatic ring having 1-4 heteroatoms independently selected from nitrogen, oxygen, and sulfur, wherein R⁴ is substituted with p instances of R⁵. In some embodiments, R⁴ is and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a 3-8 membered saturated or partially unsaturated monocyclic carbocyclic ring. In some embodiments, R⁴ is a 7-12 membered saturated or partially unsaturated bicyclic carbocyclic ring. In some embodiments, R⁴ is a phenyl. In some embodiments, R⁴ is an 8-10 membered bicyclic aromatic carbocyclic ring wherein R⁴ is substituted with p instances of R⁵. In some embodiments, R⁴ is a 3-8 membered saturated or partially unsaturated monocyclic heterocyclic ring having 1-4 heteroatoms independently selected from nitrogen, oxygen, and sulfur. In some embodiments, R⁴ is a 7-12 membered saturated or partially unsaturated bicyclic heterocyclic ring having 1-4 heteroatoms independently selected from nitrogen, oxygen, and sulfur. In some embodiments, R⁴ is a 5-6 membered monocyclic heteroaromatic ring having 1-4 heteroatoms independently selected from nitrogen, oxygen, and sulfur. In some embodiments, R⁴ is and an 8-10 membered bicyclic heteroaromatic ring having 1-5 heteroatoms independently selected from nitrogen, oxygen, and sulfur.

In some embodiments, R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, wherein R⁴ is substituted with p instances of R⁵.

In some embodiments, R⁴ is a cyclic group selected a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 7-12 membered saturated or partially unsaturated bicyclic carbocyclic ring, an 8-10 membered bicyclic aromatic carbocyclic ring, a 7-12 membered saturated or partially unsaturated bicyclic heterocyclic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 3-8 membered saturated or partially unsaturated monocyclic carbocyclic ring, phenyl, an 8-10 membered bicyclic aromatic carbocyclic ring, a 5-6 membered monocyclic heteroaromatic ring having 1-4 heteroatoms independently selected from nitrogen, oxygen, and sulfur, wherein R⁴ is substituted with p instances of R⁵.

In some embodiments, R⁴ is a cyclic group selected from a 3-6 membered saturated or partially unsaturated monocyclic carbocyclic ring, a 8-10 membered saturated or partially unsaturated bicyclic carbocyclic ring, phenyl, an 8-10 membered bicyclic aromatic carbocyclic ring, a 3-6 membered saturated or partially unsaturated monocyclic heterocyclic ring having 1-4 heteroatoms independently selected from nitrogen, oxygen, and sulfur, a 8-10 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 3-6 membered saturated or partially unsaturated monocyclic carbocyclic ring, a 8-10 membered saturated or partially unsaturated bicyclic carbocyclic ring, phenyl, an 8-10 membered bicyclic aromatic carbocyclic ring, a 3-6 membered saturated or partially unsaturated monocyclic heterocyclic ring having 2-4 heteroatoms independently selected from nitrogen, oxygen, and sulfur, a 8-10 membered saturated or partially unsaturated bicyclic heterocyclic ring having 2-4 heteroatoms independently selected from nitrogen, oxygen, and sulfur, a 5-6 membered monocyclic heteroaromatic ring having 2-4 heteroatoms independently selected from nitrogen, oxygen, and sulfur, and an 8-10 membered bicyclic heteroaromatic ring having 2-5 heteroatoms independently selected from nitrogen, oxygen, and sulfur, wherein R⁴ is substituted with p instances of R⁵.

In some embodiments, R⁴ is selected from those depicted in Table 1, below.

As defined generally above, R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence halo. In certain embodiments, R⁵ represents independently for each occurrence —OR. In certain embodiments, R¹ represents independently for each occurrence —SR. In certain embodiments, R⁵ represents independently for each occurrence —NR₂. In certain embodiments, R⁵ represents independently for each occurrence —S(O)₂R. In certain embodiments, R⁵ represents independently for each occurrence —S(O)₂NR₂. In certain embodiments, R⁵ represents independently for each occurrence —S(O)R. In certain embodiments, R⁵ represents independently for each occurrence —S(O)NR₂. In certain embodiments, R⁵ represents independently for each occurrence —C(O)R. In certain embodiments, R⁵ represents independently for each occurrence —C(O)CR₃. In certain embodiments, R⁵ represents independently for each occurrence —C(O)OR. In certain embodiments, R⁵ represents independently for each occurrence —C(O)NR₂. In certain embodiments, R¹ represents independently for each occurrence —C(O)N(R)OR. In certain embodiments, R⁵ represents independently for each occurrence —OC(O)R. In certain embodiments, R⁵ represents independently for each occurrence —OC(O)NR₂. In certain embodiments, R⁵ represents independently for each occurrence —O(CR₂)₃NR₂. In certain embodiments, R¹ represents independently for each occurrence —N(R)C(O)OR. In certain embodiments, R⁵ represents independently for each occurrence —N(R)C(O)R. In certain embodiments, R⁵ represents independently for each occurrence —N(R)C(O)NR₂. In certain embodiments, R⁵ represents independently for each occurrence —N(R)C(NR)NR₂. In certain embodiments, R⁵ represents independently for each occurrence-NR(CR₂)₂OR. In certain embodiments, R⁵ represents independently for each occurrence —N(R)S(O)₂NR₂. In certain embodiments, R⁵ represents independently for each occurrence —N(R)S(O)₂R. In certain embodiments, R⁵ represents independently for each occurrence —CH₂R. In certain embodiments, R⁵ represents independently for each occurrence —CHR₂. In certain embodiments, R⁵ represents independently for each occurrence —CR₃. In certain embodiments, R⁵ represents independently for each occurrence —CH₂NR₂. In certain embodiments, R⁵ represents independently for each occurrence —CH₂NHCH₂CH₂OR. In certain embodiments, R⁵ represents independently for each occurrence —CH₂CH₂C(O)OR. In certain embodiments, R⁵ represents independently for each occurrence —CH₂C(O)NR₂. In certain embodiments, R⁵ represents independently for each occurrence —CH₂CH₂R, In certain embodiments, R⁵ represents independently for each occurrence —CH₂CH₂OR. In certain embodiments, R⁵ represents independently for each occurrence —NHCH₂CH₂OR. In certain embodiments, R⁵ represents independently for each occurrence —NHC(O)R. In certain embodiments, R⁵ represents independently for each occurrence —CH₂OR. In certain embodiments, R⁵ represents independently for each occurrence —CH₂NHC(O)OCR₃. In certain embodiments, R⁵ represents independently for each occurrence C₁₋₃ alkyl group. In certain embodiments, R⁵ represents independently for each occurrence a C₁₋₃ haloalkyl group. In certain embodiments, R⁵ represents independently for each occurrence a C₁₋₃ heteroalkyl group having 1-2 heteroatoms independently selected from nitrogen, oxygen, and sulfur. In certain embodiments, R⁵ represents independently for each occurrence a 3-6 membered saturated or partially unsaturated monocyclic carbocyclic ring. In certain embodiments, R⁵ represents independently for each occurrence phenyl. In certain embodiments, R⁵ represents independently for each occurrence a 5-6 membered monocyclic heteroaromatic ring having 1-4 heteroatoms independently selected from nitrogen, oxygen, and sulfur. In certain embodiments, R⁵ represents independently for each occurrence a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence oxo, —OR, —OC(O)R, —OC(O)NR₂, or —O(CR₂)₃NR₂.

In certain embodiments, R⁵ represents independently for each occurrence —CN, —C(O)R, —C(O)CR₃, —C(O)OR, —C(O)NR₂, —C(O)N(R)OR, —CR₃, —CH₂NR₂, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In certain embodiments, R⁵ represents independently for each occurrence a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, or a C₁₋₃ haloalkyl group.

In certain embodiments, R⁵ represents independently for each occurrence halo, oxo, —CN, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence halo, oxo, a C₁₋₃ alkyl group, a C₁₋₃ haloalkyl group, or a C₁₋₃ heteroalkyl group having 1-2 heteroatoms independently selected from nitrogen, oxygen, and sulfur.

In certain embodiments, R⁵ is halo. In certain embodiments, R⁵ is —OR. In certain embodiments, R⁵ is —SR. In certain embodiments, R⁵ is —NR₂. In certain embodiments, R⁵ is —S(O)₂R. In certain embodiments, R⁵ is —S(O)₂NR₂. In certain embodiments, R⁵ is —S(O)R. In certain embodiments, R⁵ is —S(O)NR₂. In certain embodiments, R⁵ is —C(O)R. In certain embodiments, R⁵ is —C(O)CR₃. In certain embodiments, R⁵ is —C(O)OR. In certain embodiments, R⁵ is —C(O)NR₂. In certain embodiments, R⁵ is —C(O)N(R)OR. In certain embodiments, R⁵ is —OC(O)R. In certain embodiments, R⁵ is —OC(O)NR₂. In certain embodiments, R⁵ is —O(CR₂)₃NR₂. In certain embodiments, R⁵ is —N(R)C(O)OR. In certain embodiments, R⁵ is —N(R)C(O)R. In certain embodiments, R⁵ is —N(R)C(O)NR₂. In certain embodiments, R⁵ is —N(R)C(NR)NR₂. In certain embodiments, R⁵ is-NR(CR₂)₂OR. In certain embodiments, R⁵ is —N(R)S(O)₂NR₂. In certain embodiments, R⁵ is —N(R)S(O)₂R. In certain embodiments, R⁵ is —CH₂R. In certain embodiments, R⁵ is —CHR₂. In certain embodiments, R⁵ is —CR₃. In certain embodiments, R⁵ is —CH₂NR₂. In certain embodiments, R⁵ is —CH₂NHCH₂CH₂OR. In certain embodiments, R⁵ is —CH₂CH₂C(O)OR. In certain embodiments, R⁵ is —CH₂C(O)NR₂. In certain embodiments, R⁵ is —CH₂CH₂R, In certain embodiments, R⁵ is —CH₂CH₂OR. In certain embodiments, R⁵ is —NHCH₂CH₂OR. In certain embodiments, R⁵ is —NHC(O)R. In certain embodiments, R⁵ is —CH₂OR. In certain embodiments, R⁵ is —CH₂NHC(O)OCR₃. In certain embodiments, R⁵ isa C₁₋₃ alkyl group. In certain embodiments, R⁵ is a C₁₋₃ haloalkyl group. In certain embodiments, R⁵ is a C₁₋₃ heteroalkyl group having 1-2 heteroatoms independently selected from nitrogen, oxygen, and sulfur. In certain embodiments, R⁵ is a 3-6 membered saturated or partially unsaturated monocyclic carbocyclic ring. In certain embodiments, R⁵ is phenyl. In certain embodiments, R⁵ is a 5-6 membered monocyclic heteroaromatic ring having 1-4 heteroatoms independently selected from nitrogen, oxygen, and sulfur. In certain embodiments, R⁵ is a 3-6 membered saturated or partially unsaturated monocyclic heterocyclic ring having 1-4 heteroatoms independently selected from nitrogen, oxygen, and sulfur.

In certain embodiments, R⁵ is oxo, —OR, —OC(O)R, —OC(O)NR₂, or —O(CR₂)₃NR₂.

In certain embodiments, R⁵ is —CN, —C(O)R, —C(O)CR₃, —C(O)OR, —C(O)NR₂, —C(O)N(R)OR, —CR₃, —CH₂NR₂—, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ is —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In certain embodiments, R⁵ is a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ is halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂—, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, or a C₁₋₃ haloalkyl group.

In certain embodiments, R⁵ is halo, oxo, —CN, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ is halo, oxo, a C₁₋₃ alkyl group, a C₁₋₃ haloalkyl group, or a C₁₋₃ heteroalkyl group having 1-2 heteroatoms independently selected from nitrogen, oxygen, and sulfur.

In some embodiments, R⁵ is selected from those depicted in Table 1, below.

As defined generally above, X is —O— or —NH—. In certain embodiments, X is —O—. In certain embodiments, X is —NH—.

In some embodiments, X is selected from those depicted in Table 1, below.

As defined generally above, R represents independently for each occurrence hydrogen, hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence halo. In certain embodiments, R represents independently for each occurrence optionally substituted C₁₋₆ aliphatic. In certain embodiments, R represents independently for each occurrence optionally substituted phenyl. In certain embodiments, R represents independently for each occurrence optionally substituted 3-7 membered saturated or partially unsaturated monocyclic heterocyclic ring having 1-4 heteroatoms independently selected from nitrogen, oxygen, and sulfur. In certain embodiments, R represents independently for each occurrence optionally substituted 5-6 membered monocyclic heteroaryl ring having 1-4 heteroatoms independently selected from nitrogen, oxygen, and sulfur. In certain embodiments, R represents independently for each occurrence optionally substituted 3-7 membered saturated or partially unsaturated monocyclic carbocyclic ring.

In certain embodiments, R represents independently for each occurrence halo. In certain embodiments, R represents independently for each occurrence C₁₋₆ aliphatic. In certain embodiments, R represents independently for each occurrence phenyl. In certain embodiments, R represents independently for each occurrence 3-7 membered saturated or partially unsaturated monocyclic heterocyclic ring having 1-4 heteroatoms independently selected from nitrogen, oxygen, and sulfur. In certain embodiments, R represents independently for each occurrence 5-6 membered monocyclic heteroaryl ring having 1-4 heteroatoms independently selected from nitrogen, oxygen, and sulfur. In certain embodiments, R represents independently for each occurrence 3-7 membered saturated or partially unsaturated monocyclic carbocyclic ring.

In certain embodiments, R represents independently for each occurrence hydrogen, hydroxyl, halo, or a group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence hydrogen, hydroxyl, halo, or optionally substituted C₁₋₆ aliphatic.

In certain embodiments, R represents independently for each occurrence an optionally substituted group selected from phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence hydrogen, hydroxyl, halo, or an optionally substituted group selected from C₁₋₃ aliphatic, phenyl, a 3-6 membered saturated or partially unsaturated monocyclic heterocyclic ring having 1-3 heteroatoms independently selected from nitrogen, oxygen, and sulfur, a 5-6 membered monocyclic heteroaryl ring having 1-3 heteroatoms independently selected from nitrogen, oxygen, and sulfur, and a 3-6 membered saturated or partially unsaturated monocyclic carbocyclic ring.

In certain embodiments, R represents independently for each occurrence hydrogen, hydroxyl, Cl, F, —CH₂OH, —CH₂CH₂OH, methyl, ethyl, i-propyl, n-propyl, cyclopropyl, or phenyl.

In certain embodiments, R is halo. In certain embodiments, R is optionally substituted C₁₋₆ aliphatic. In certain embodiments, R is optionally substituted phenyl. In certain embodiments, R is optionally substituted 3-7 membered saturated or partially unsaturated monocyclic heterocyclic ring having 1-4 heteroatoms independently selected from nitrogen, oxygen, and sulfur. In certain embodiments, R is optionally substituted 5-6 membered monocyclic heteroaryl ring having 1-4 heteroatoms independently selected from nitrogen, oxygen, and sulfur. In certain embodiments, R is optionally substituted 3-7 membered saturated or partially unsaturated monocyclic carbocyclic ring.

In certain embodiments, R is halo. In certain embodiments, R is C₁₋₆ aliphatic. In certain embodiments, R is phenyl. In certain embodiments, R is 3-7 membered saturated or partially unsaturated monocyclic heterocyclic ring having 1-4 heteroatoms independently selected from nitrogen, oxygen, and sulfur. In certain embodiments, R is 5-6 membered monocyclic heteroaryl ring having 1-4 heteroatoms independently selected from nitrogen, oxygen, and sulfur. In certain embodiments, R is 3-7 membered saturated or partially unsaturated monocyclic carbocyclic ring.

In certain embodiments, R is hydrogen, hydroxyl, halo, or a group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R is hydrogen, hydroxyl, halo, or optionally substituted C₁₋₆ aliphatic.

In certain embodiments, R is an optionally substituted group selected from phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R is hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R is hydrogen, hydroxyl, halo, or an optionally substituted group selected from C₁₋₃ aliphatic, phenyl, a 3-6 membered saturated or partially unsaturated monocyclic heterocyclic ring having 1-3 heteroatoms independently selected from nitrogen, oxygen, and sulfur, a 5-6 membered monocyclic heteroaryl ring having 1-3 heteroatoms independently selected from nitrogen, oxygen, and sulfur, and a 3-6 membered saturated or partially unsaturated monocyclic carbocyclic ring.

In certain embodiments, R is hydrogen, hydroxyl, Cl, F, —CH₂OH, —CH₂CH₂OH, methyl, ethyl, i-propyl, n-propyl, cyclopropyl, or phenyl.

In some embodiments, R is selected from those depicted in Table 1, below.

As defined generally above, n is 0, 1, 2, or 3. In certain embodiments, n is 1, 2, or 3. In certain embodiments, n is 0, 2, or 3. In certain embodiments, n is 0, 1, or 3. In certain embodiments, n is 0, 1, or 2. In certain embodiments, n is 2 or 3. In certain embodiments, n is 0 or 1. In certain embodiments, n is 0 or 3. In certain embodiments, n is 0 or 2. In certain embodiments, n is 1 or 2. In certain embodiments, n is 1 or 3. In certain embodiments, n is 0. In certain embodiments, n is 1. In certain embodiments, n is 2. In certain embodiments, n is 3.

As defined generally above, p is 0, 1, 2, 3 or 4. In certain embodiments, p is 1, 2, 3 or 4. In certain embodiments, p is 0, 2, 3 or 4. In certain embodiments, p is 0, 1, 3 or 4. In certain embodiments, p is 0, 1, 2, or 4. In certain embodiments, p is 0, 1, 2, or 3. In certain embodiments, p is 2, 3 or 4. In certain embodiments, p is 0, 3 or 4. In certain embodiments, p is 0, 1, or 4. In certain embodiments, p is 0, 1, or 2. In certain embodiments, p is 1, 3 or 4. In certain embodiments, p is 1, 2, or 4. In certain embodiments, p is 1, 2, or 3. In certain embodiments, p is 0, 2 or 4. In certain embodiments, p is 0, 2 or 3. In certain embodiments, p is 0, 1 or 3. In certain embodiments, p is 3 or 4. In certain embodiments, p is 0 or 4. In certain embodiments, p is 0 or 1. In certain embodiments, p is 1 or 4. In certain embodiments, p is 1 or 2. In certain embodiments, p is 0 or 2. In certain embodiments, p is 0 or 3. In certain embodiments, p is 1 or 3. In certain embodiments, p is 2 or 3. In certain embodiments, p is 2 or 4. In certain embodiments, p is 0. In certain embodiments, p is 1. In certain embodiments, p is 2. In certain embodiments, p is 3. In certain embodiments, p is 4.

One aspect of the invention provides a compound represented by Formula IV:

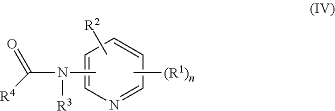

or a pharmaceutically acceptable salt thereof, wherein:
Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur, a 4-8 membered saturated or partially unsaturated monocyclic heterocyclic ring containing 1-4 heteroatoms independently selected from nitrogen, oxygen, and sulfur, a 5-6 membered monocyclic heteroaromatic ring having 1-4 heteroatoms independently selected from nitrogen, oxygen, and sulfur, a 6-11 membered saturated or partially unsaturated bicyclic carbocyclic ring, a 3-8 membered saturated or partially unsaturated monocyclic carbocyclic ring, an 8-10 membered bicyclic aromatic carbocyclic ring, or phenyl;
R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, C₁₋₄ haloaliphatic, or a cyclic group selected from a 3-8 membered saturated monocyclic carbocyclic ring or a 3-8 membered saturated monocyclic heterocyclic ring having 1-4 heteroatoms independently selected from nitrogen, oxygen, and sulfur;
R² is hydrogen, halo, —OH, —CN, cyclopropyl. C₁₋₃ alkyl optionally substituted with a group selected from —OH, —NH₂, —NH(CH₃), and —N(CH₃)₂, C₁₋₃ haloalkyl, or C₁₋₃ alkoxy;
R³ is hydrogen, cyclopropyl, or C₁₋₃ alkyl optionally substituted with a group selected from —OH, —OMe, —NH₂, —NH(CH₃), and —N(CH₃)₂;
R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵;
R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur;
R represents independently for each occurrence hydrogen, hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring;
n is 0, 1, 2, or 3; and
p is 0, 1, 2, 3 or 4.

The definitions of variables in Formula IV above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula IV.

As defined generally above, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur; a 5-6 membered monocyclic heteroaromatic ring having 1-4 heteroatoms independently selected from nitrogen, oxygen, and sulfur; a 6-11 membered saturated or partially unsaturated bicyclic carbocyclic ring; a 3-8 membered saturated or partially unsaturated monocyclic carbocyclic ring; an 8-10 membered bicyclic aromatic carbocyclic ring; or phenyl.

In some embodiments, Ring A is a 6-14 membered saturated or partially unsaturated bicyclic heterocyclic ring containing 1-4 heteroatoms independently selected from nitrogen, oxygen, and sulfur. In some embodiments, Ring A is an 8-10 membered bicyclic heteroaromatic ring having 1-4 heteroatoms independently selected from nitrogen, oxygen, and sulfur. In some embodiments, Ring A is a 4-8 membered saturated or partially unsaturated monocyclic heterocyclic ring containing 1-4 heteroatoms independently selected from nitrogen, oxygen, and sulfur. In some embodiments, Ring A is a 5-6 membered monocyclic heteroaromatic ring having 1-4 heteroatoms independently selected from nitrogen, oxygen, and sulfur. In some embodiments, Ring A is a 6-11 membered saturated or partially unsaturated bicyclic carbocyclic ring; a 3-8 membered saturated or partially unsaturated monocyclic carbocyclic ring. In some embodiments, Ring A is an 8-10 membered bicyclic aromatic carbocyclic ring. In some embodiments, Ring A is phenyl.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur or a 5-6 membered monocyclic heteroaromatic ring having 1-4 heteroatoms independently selected from nitrogen, oxygen, and sulfur.

In some embodiments, Ring A is a 6-14 membered saturated or partially unsaturated bicyclic heterocyclic ring containing 1-4 heteroatoms independently selected from nitrogen, oxygen, and sulfur; or an 8-10 membered bicyclic heteroaromatic ring having 1-4 heteroatoms independently selected from nitrogen, oxygen, and sulfur.

In some embodiments, Ring A is a 4-8 membered saturated or partially unsaturated monocyclic heterocyclic ring containing 1-4 heteroatoms independently selected from nitrogen, oxygen, and sulfur or a 5-6 membered monocyclic heteroaromatic ring having 1-4 heteroatoms independently selected from nitrogen, oxygen, and sulfur.

In some embodiments, Ring A is a 6-11 membered saturated or partially unsaturated bicyclic carbocyclic ring; a 3-8 membered saturated or partially unsaturated monocyclic carbocyclic ring; an 8-10 membered bicyclic aromatic carbocyclic ring; or phenyl.

In some embodiments, Ring A is a 3-8 membered saturated or partially unsaturated monocyclic carbocyclic ring or phenyl.

In some embodiments, Ring A is a 6-11 membered saturated or partially unsaturated bicyclic carbocyclic ring; or an 8-10 membered bicyclic aromatic carbocyclic ring.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur; a 5-6 membered monocyclic heteroaromatic ring having 1-4 heteroatoms independently selected from nitrogen, oxygen, and sulfur; or a 6-11 membered saturated or partially unsaturated bicyclic carbocyclic ring; a 3-8 membered saturated or partially unsaturated monocyclic carbocyclic ring.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur; a 4-8 membered saturated or partially unsaturated monocyclic heterocyclic ring containing 1-4 heteroatoms independently selected from nitrogen, oxygen, and sulfur; a 5-6 membered monocyclic heteroaromatic ring having 1-4 heteroatoms independently selected from oxygen, and sulfur; a 6-11 membered saturated or partially unsaturated bicyclic carbocyclic ring; a 3-8 membered saturated or partially unsaturated monocyclic carbocyclic ring; or phenyl.

In some embodiments, Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur: a 4-8 membered saturated or partially unsaturated monocyclic heterocyclic ring containing 1-4 heteroatoms independently selected from nitrogen, oxygen, and sulfur; a 6-11 membered saturated or partially unsaturated bicyclic carbocyclic ring; a 3-8 membered saturated or partially unsaturated monocyclic carbocyclic ring; or phenyl.

In some embodiments, Ring A is selected from those depicted in Table 1, below.

As defined generally above, R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ represents independently for each occurrence halo. In some embodiments, R¹ represents independently for each occurrence —OR. In some embodiments. R¹ represents independently for each occurrence —SR. In some embodiments. R¹ represents independently for each occurrence —NR₂. In some embodiments, R¹ represents independently for each occurrence —S(O)₂R. In some embodiments, R¹ represents independently for each occurrence —S(O)₂NR₂. In some embodiments, R¹ represents independently for each occurrence —S(O)R. In some embodiments, R¹ represents independently for each occurrence —S(O)NR₂. In some embodiments, R¹ represents independently for each occurrence —C(O)R. In some embodiments, R¹ represents independently for each occurrence —C(O)CR₃. In some embodiments, R¹ represents independently for each occurrence —C(O)OR. In some embodiments, R¹ represents independently for each occurrence —C(O)NR₂. In some embodiments, R¹ represents independently for each occurrence —C(O)N(R)OR. In some embodiments, R¹ represents independently for each occurrence —OC(O)R. In some embodiments, R¹ represents independently for each occurrence —OC(O)NR₂. In some embodiments, R¹ represents independently for each occurrence —O(CR₂)₃NR₂. In some embodiments, R¹ represents independently for each occurrence —N(R)C(O)OR. In some embodiments, R¹ represents independently for each occurrence —N(R)C(O)R. In some embodiments, R¹ represents independently for each occurrence —N(R)C(O)NR₂. In some embodiments, R¹ represents independently for each occurrence —N(R)C(NR)NR₂. In some embodiments, R¹ represents independently for each occurrence —NR(CR₂)₂OR. In some embodiments, R¹ represents independently for each occurrence —N(R)S(O)₂NR₂. In some embodiments, R¹ represents independently for each occurrence —N(R)S(O)₂R. In some embodiments, R¹ represents independently for each occurrence —CH₂R. In some embodiments, R¹ represents independently for each occurrence —CHR₂. In some embodiments, R¹ represents independently for each occurrence —CR₃. In some embodiments. R¹ represents independently for each occurrence —CH₂NR₂. In some embodiments, R¹ represents independently for each occurrence —CH₂NHCH₂CH₂OR. In some embodiments, R¹ represents independently for each occurrence —CH₂CH₂C(O)OR. In some embodiments, R¹ represents independently for each occurrence —CH₂C(O)NR₂. In some embodiments, R¹ represents independently for each occurrence —CH₂CH₂R. In some embodiments, R¹ represents independently for each occurrence —CH₂CH₂OR. In some embodiments, R¹ represents independently for each occurrence —NHCH₂CH₂OR. In some embodiments, R¹ represents independently for each occurrence —NHC(O)R. In some embodiments, R¹ represents independently for each occurrence —CH₂OR. In some embodiments, R¹ represents independently for each occurrence —CH₂C(O)NH₂. In some embodiments, R¹ represents independently for each occurrence —CH₂NHC(O)OCR₃. In some embodiments, R¹ represents independently for each occurrence —OCR. In some embodiments, R¹ represents independently for each occurrence C₁₋₄ aliphatic. In some embodiments, R¹ represents independently for each occurrence C₁₋₄ haloaliphatic. In some embodiments, R¹ represents independently for each occurrence a 3-8 membered saturated monocyclic carbocyclic ring. In some embodiments, R¹ represents independently for each occurrence a 3-8 membered saturated monocyclic heterocyclic ring having 1-4 heteroatoms independently selected from nitrogen, oxygen, and sulfur.

In some embodiments, R¹ represents independently for each occurrence —CN, —C(O)R, —C(O)CR₃, —C(O)OR, —C(O)NR₂, —C(O)N(R)OR, —CR₃, —CH₂NR₂—, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ represents independently for each occurrence halo, oxo, —OR, —SR, —NR₂, —S(O)₂R, —S(O)₂NR₂, —S(O)R, —S(O)NR₂, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, —NHC(O)R, or —OCR.

In some embodiments, R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, or C₁₋₄ haloaliphatic.

In some embodiments, R¹ represents independently for each occurrence halo, oxo, —CN, C₁₋₄ aliphatic. Cia haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ represents independently for each occurrence oxo, —OR, —OC(O)R, —OC(O)NR₂, —O(CR₂)₃NR₂, or —OCR.

In some embodiments, R¹ represents independently for each occurrence —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In some embodiments, R¹ represents independently for each occurrence halo, oxo, —OR, —C(O)R, —C(O)CR₃, —C(O)OR, —N(R)C(O)OR, —N(R)C(O)R, —N(R)C(O)NR₂, —CR₃, —CH₂OR, —CH₂C(O)NH₂, or C₁₋₄ aliphatic.

In some embodiments, R¹ represents independently for each occurrence oxo or C₁₋₄ aliphatic.

In some embodiments, R¹ represents independently for each occurrence —CH₃, —CH₂C(O)NH₂, —CH₂OCH₃, —CH₂CH₂OCH₃, —NHAc, —OH, —C(O)CH₂OH, —C(O)CH₃, —CH₂NHCH₂CH₂OH, —CH₂C(O)NH₂, —OCH₃, CH₂OCH₃, —C(O)OCH₃, —C(O)OH, —NHCH₂CH₂OH, —CH₂CH₂C(O)OCH₃, —NHS(O)₂CH₃, —C(O)NH₂, —S(O)₂CH₃, -Bn, —OC(CH₃)₂, —NHCH₂CH₂OCH₃, —F, —Cl, —Br, (O), —CH₂NH₂, —NH₂, —NHC(O)CH₃, —C(O)OCH₂CH₃, —S(O)₂NHCH₂CH₂OH, —CH₂CH₃, —N(CH₃)₂, —CH(CH₃)₂, —CF₃, —CN, —S(O)₂CH₃, —CH₂OH, —OCH₃, —CH₂N(CH₃)₂, —O(CH₂)₃N(CH₃)₂, —C(O)NH₂, -cyclohexyl, —OCF₃, —S(O)₂NH₂, —CHF₂, —CH₂NHC(O)OC(CH₃)₃, —C(CH₃)₂(OH), —CH(OH)CH₃, —O(CH₂)₂NH₂,

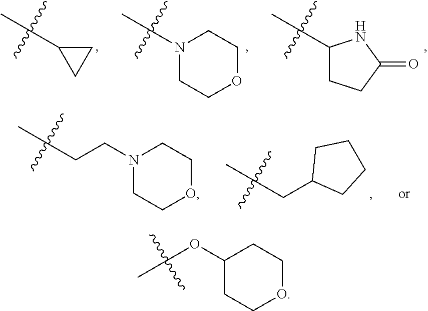

In some embodiments, R¹ represents independently for each occurrence —CH₃, —NHAc, —OH, —C(O)CH₂OH, —C(O)CH₃, —C(O)OCH₃, —C(O)OH, —C(O)NH₂, (O), —CH₂NH₂, —NH₂, —NHC(O)CH₃, —CH₂CH₃, —CH₂OH, —OCH₃, or

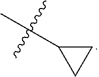 [SMILES: *C1CC1]

In some embodiments, R¹ represents independently for each occurrence —CH₃, —NHAc, —OH, (O), —OCH₃,

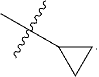 [SMILES: *C1CC1]

In some embodiments, R¹ represents independently for each occurrence —CH₃ or (O).

In some embodiments, R¹ is halo. In some embodiments, R¹ is —OR. In some embodiments, R¹ is —SR. In some embodiments, R¹ is —NR₂. In some embodiments, R¹ is —S(O)₂R. In some embodiments, R¹ is —S(O)₂NR₂. In some embodiments, R¹ is —S(O)R. In some embodiments, R¹ is —S(O)NR₂. In some embodiments, R¹ is —C(O)R. In some embodiments, R¹ is —C(O)CR₃. In some embodiments, R¹ is —C(O)OR. In some embodiments, R¹ is —C(O)NR₂. In some embodiments, R¹ is —C(O)N(R)OR. In some embodiments, R¹ is —OC(O)R. In some embodiments, R¹ is —OC(O)NR₂. In some embodiments, R¹ is —O(CR₂)₃NR₂. In some embodiments, R¹ is —N(R)C(O)OR. In some embodiments, R¹ is —N(R)C(O)R. In some embodiments, R¹ is —N(R)C(O)NR₂. In some embodiments, R¹ is —N(R)C(NR)NR₂. In some embodiments, R¹ is —NR(CR₂)₂OR. In some embodiments, R¹ is —N(R)S(O)₂NR₂. In some embodiments, R¹ is —N(R)S(O)₂R. In some embodiments, R¹ is —CR₃. In some embodiments. R¹ is —CH₂NR₂. In some embodiments, R¹ is —CH₂NHCH₂CH₂OR. In some embodiments, R¹ is —CH₂CH₂C(O)OR. In some embodiments, R¹ is —CH₂C(O)NR₂. In some embodiments, R¹ is —CH₂CH₂R. In some embodiments, R¹ is —CH₂CH₂OR. In some embodiments, R¹ is —NHCH₂CH₂OR. In some embodiments, R¹ is —NHC(O)R. In some embodiments, R¹ is —CH₂OR. In some embodiments, R¹ is —CH₂C(O)NH₂. In some embodiments, R¹ is —CH₂NHC(O)OCR₃. In some embodiments, R¹ is —OCR. In some embodiments, R¹ is C₁₋₄ aliphatic. In some embodiments, R¹ is C₁₋₄ haloaliphatic. In some embodiments, R¹ is a 3-8 membered saturated monocyclic carbocyclic ring. In some embodiments, R¹ is a 3-8 membered saturated monocyclic heterocyclic ring having 1-4 heteroatoms independently selected from nitrogen, oxygen, and sulfur.

In some embodiments, R¹ is —CN, —C(O)R, —C(O)CR₃, —C(O)OR, —C(O)NR₂, —C(O)N(R)OR, —CR₃, —CH₂NR₂, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ is halo, oxo, —OR, —SR, —NR₂, —S(O)₂R, —S(O)₂NR₂, —S(O)R, —S(O)NR₂, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, —NHC(O)R, or —OCR.

In some embodiments, R¹ is halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, or C₁₋₄ haloaliphatic.

In some embodiments, R¹ is halo, oxo, —CN, C₁₋₄ aliphatic, C₁₋₄ haloaliphatic, or a cyclic group selected from a 3-8 membered saturated monocyclic carbocyclic ring and a 3-8 membered saturated monocyclic heterocyclic ring having 1-4 heteroatoms independently selected from nitrogen, oxygen, and sulfur.

In some embodiments, R¹ is oxo, —OR, —OC(O)R, —OC(O)NR₂, —O(CR₂)₃NR₂, or —OCR.

In some embodiments, R¹ is —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In some embodiments, R¹ is halo, oxo, —OR, —C(O)R, —C(O)CR₃, —C(O)OR, —N(R)C(O)OR, —N(R)C(O)R, —N(R)C(O)NR₂, —CR₃, —CH₂OR, —CH₂C(O)NH₂, or C₁₋₄ aliphatic.

In some embodiments, R¹ is oxo or C₁₋₄ aliphatic.

In some embodiments, R¹ is —CH₃, —CH₂C(O)NH₂, —CH₂OCH₃, —CH₂CH₂OCH₃, —NHAc, —OH, —C(O)CH₂OH, —C(O)CH₃, —CH₂NHCH₂CH₂OH, —CH₂C(O)NH₂, —OCH₃, CH₂OCH₃, —C(O)OCH₃, —C(O)OH, —NHCH₂CH₂OH, —CH₂CH₂C(O)OCH₃, —NHS(O)₂CH₃, —C(O)NH₂, —S(O)₂CH₃, -Bn, —OC(CH₃)₂, —NHCH₂CH₂OCH₃, —F, —Cl, —Br, (O), —CH₂NH₂, —NH₂, —NHC(O)CH₃, —C(O)OCH₂CH₃, —S(O)₂NHCH₂CH₂OH, —CH₂CH₃, —N(CH₃)₂, —CH(CH₃)₂, —CF₃, —CN, —S(O)₂CH₃, —CH₂OH, —OCH₃, —CH₂N(CH₃)₂, —O(CH₂)₃N(CH₃)₂, —C(O)NH₂, -cyclohexyl, —OCF₃, —S(O)₂NH₂, —CHF₂, —CH₂NHC(O)OC(CH₃)₃, —C(CH₃)₂(OH), —CH(OH)CH₃, —O(CH₂)₂NH₂,

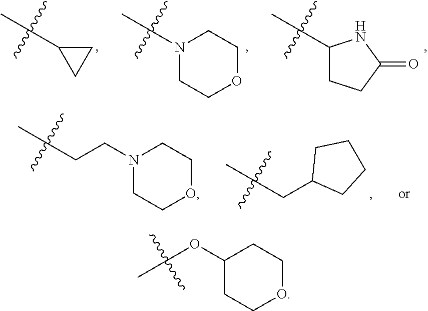

In some embodiments, R¹ is —CH₃, —NHAc, —OH, —C(O)CH₂OH, —C(O)CH₃, —C(O)OCH₃, —C(O)OH, —C(O)NH₂, (O), —CH₂NH₂, —NH₂, —NHC(O)CH₃, —CH₂CH₃, —CH₂OH, —OCH₃, or

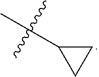 [SMILES: *C1CC1]

In some embodiments, R¹ is —CH₃, —NHAc, —OH, (O), —OCH₃, or

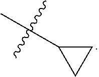 [SMILES: *C1CC1]

In some embodiments, R¹ is —CH₃ or (O).

In some embodiments, R¹ is selected from those depicted in Table 1, below.

As defined generally above, R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy.

In some embodiments, R² is halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy. In some embodiments, R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy. In some embodiments, R² is hydrogen, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy. In some embodiments, R² is hydrogen, halo, —CN, cyclopropyl, C₁₋₃ alkyl, C₁₋₃ haloalkyl, or C₁₋₃ alkoxy. In some embodiments, R² is hydrogen, halo, —OH, cyclopropyl, C₁₋₃ alkyl, C₁₋₃ haloalkyl, or C₁₋₃ alkoxy. In some embodiments, R² is hydrogen, halo, —OH, —CN, C₁₋₃ alkyl, C₁₋₃ haloalkyl, or C₁₋₃ alkoxy. In some embodiments, R² is hydrogen, halo, —OH, —CN, cyclopropyl, C₁₋₃ haloalkyl, or C₁₋₃ alkoxy. In some embodiments, R² is hydrogen, halo, —OH, —CN, cyclopropyl. C₁₋₃ alkyl, or C₁₋₃ alkoxy. In some embodiments, R² is hydrogen, halo, —OH, —CN, cyclopropyl, C₁₋₃ alkyl, or C₁₋₃ haloalkyl.

In some embodiments, R² is hydrogen, cyclopropyl. C₁₋₃ alkyl optionally substituted with a group selected from —OH, —NH₂, —NH(CH₃), and —N(CH₃)₂, or C₁₋₃ alkoxy. In some embodiments, R² is hydrogen or C₁₋₃ alkyl optionally substituted with a group selected from —OH, —NH₂, —NH(CH₃), and —N(CH₃)₂. In some embodiments, R² is hydrogen or C₁₋₃ alkyl. In some embodiments, R² unsubstituted C₁₋₃ alkyl. In some embodiments, R² is C₁₋₃ alkyl substituted with a group selected from —OH, —NH₂, —NH(CH₃), and —N(CH₃)₂.

In some embodiments, R² is hydrogen. In some embodiments. R² is —OH. In some embodiments, R² is —CN. In some embodiments, R² is cyclopropyl.

In some embodiments, R² is halo. In some embodiments, R² is Cl, F, or Br. In some embodiments, R² is Cl. In some embodiments, R² is F. In some embodiments, R² is Br.

In some embodiments, R² is C₁₋₃ alkyl. In some embodiments, R² is methyl. In some embodiments, R² is ethyl. In some embodiments, R² is i-propyl. In some embodiments, R² is n-propyl.

In some embodiments, R² is C₁₋₃ haloalkyl. In some embodiments, R² is CF₃. In some embodiments, R² is CHF₂.

In some embodiments, R² is C₁₋₃ alkoxy. In some embodiments. R² is —CH₂OCH₃. In some embodiments, R₂ is —CH₂OCH₃. In some embodiments, R₂ is —CH₂CH₂OCH₃.

In some embodiments, R² is selected from those depicted in Table 1, below.

As defined generally above, R³ is hydrogen, cyclopropyl, or C₁₋₃ alkyl optionally substituted with a group selected from —OH, OMe, —NH₂, —NH(CH₃), and —N(CH₃)₂.

In some embodiments, R³ is cyclopropyl, or C₁₋₃ alkyl optionally substituted with a group selected from —OH, —OMe, —NH₂, —NH(CH₃), and —N(CH₃)₂. In some embodiments, R³ is hydrogen, cyclopropyl, or C₁₋₃ alkyl. In some embodiments, R³ is hydrogen or C₁₋₃ alkyl optionally substituted with a group selected from —OH, —OMe, —NH₂, —NH(CH₃), and —N(CH₃)₂. In some embodiments, R³ is hydrogen, cyclopropyl, or C₁₋₃ alkyl substituted with a group selected from —OH, —OMe, —NH₂, —NH(CH₃), and —N(CH₃)₂. In some embodiments, R³ is substituted C₁₋₃ alkyl as defined above and herein, wherein the substitution occurs on the terminal carbon atom.

In some embodiments, R³ is hydrogen or C₁₋₃ alkyl optionally substituted with a group selected from —OH and OMe. In some embodiments. R³ is hydrogen or C₁₋₃ alkyl.

In some embodiments, R³ is hydrogen. In some embodiments. R³ is cyclopropyl. In some embodiments, R³ is C₁₋₃ alkyl. In some embodiments, R³ is methyl. In some embodiments, R³ is ethyl. In some embodiments, R³ is i-propyl. In some embodiments, R³ is n-propyl.

In some embodiments, R³ is C₁₋₃ alkyl substituted with a group selected from —OH, OMe, —NH₂, —NH(CH₃), and —N(CH₃)₂. In some embodiments, R³ is C₁₋₃ alkyl substituted with a group selected from —OH or OMe. In some embodiments, R³ is C₁₋₃ alkyl substituted with a group selected from —OMe, —NH₂, —NH(CH₃), or —N(CH₃)₂. In some embodiments, R³ is C₁₋₃ alkyl substituted with a group selected from —NH₂, —NH(CH₃), or —N(CH₃)₂. In some embodiments, R³ is —CH₂OCH₃. In some embodiments, R³ is —CH₂OCH₃. In some embodiments, R³ is —CH₂CH₂OCH₃.

In some embodiments, R³ is selected from those depicted in Table 1, below.

As defined generally above, R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments. R⁴ is a 3-8 membered saturated or partially unsaturated monocyclic carbocyclic ring, wherein R⁴ is substituted with p instances of R⁵. In some embodiments, R⁴ is a 7-12 membered saturated or partially unsaturated bicyclic carbocyclic ring, wherein R⁴ is substituted with p instances of R⁵. In some embodiments, R⁴ is a phenyl, wherein R⁴ is substituted with p instances of R⁵. In some embodiments, R⁴ is an 8-10 membered bicyclic aromatic carbocyclic ring wherein R⁴ is substituted with p instances of R⁵. In some embodiments, R⁴ is a 3-8 membered saturated or partially unsaturated monocyclic heterocyclic ring having 1-4 heteroatoms independently selected from nitrogen, oxygen, and sulfur, wherein R⁴ is substituted with p instances of R⁵. In some embodiments. R⁴ is a 7-12 membered saturated or partially unsaturated bicyclic heterocyclic ring having 1-4 heteroatoms independently selected from nitrogen, oxygen, and sulfur, wherein R⁴ is substituted with p instances of R⁵. In some embodiments, R⁴ is a 5-6 membered monocyclic heteroaromatic ring having 1-4 heteroatoms independently selected from nitrogen, oxygen, and sulfur, wherein R⁴ is substituted with p instances of R⁵. In some embodiments, R⁴ is and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a 3-8 membered saturated or partially unsaturated monocyclic carbocyclic ring. In some embodiments, R⁴ is a 7-12 membered saturated or partially unsaturated bicyclic carbocyclic ring. In some embodiments, R⁴ is a phenyl. In some embodiments. R⁴ is an 8-10 membered bicyclic aromatic carbocyclic ring wherein R⁴ is substituted with p instances of R⁵. In some embodiments, R⁴ is a 3-8 membered saturated or partially unsaturated monocyclic heterocyclic ring having 1-4 heteroatoms independently selected from nitrogen, oxygen, and sulfur. In some embodiments, R⁴ is a 7-12 membered saturated or partially unsaturated bicyclic heterocyclic ring having 1-4 heteroatoms independently selected from nitrogen, oxygen, and sulfur. In some embodiments, R⁴ is a 5-6 membered monocyclic heteroaromatic ring having 1-4 heteroatoms independently selected from nitrogen, oxygen, and sulfur. In some embodiments, R⁴ is and an 8-10 membered bicyclic heteroaromatic ring having 1-5 heteroatoms independently selected from nitrogen, oxygen, and sulfur.

In some embodiments, R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, wherein R⁴ is substituted with p instances of R⁵.

In some embodiments, R⁴ is a cyclic group selected a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 7-12 membered saturated or partially unsaturated bicyclic carbocyclic ring, an 8-10 membered bicyclic aromatic carbocyclic ring, a 7-12 membered saturated or partially unsaturated bicyclic heterocyclic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 3-8 membered saturated or partially unsaturated monocyclic carbocyclic ring, phenyl, an 8-10 membered bicyclic aromatic carbocyclic ring, a 5-6 membered monocyclic heteroaromatic ring having 1-4 heteroatoms independently selected from nitrogen, oxygen, and sulfur, wherein R⁴ is substituted with p instances of R⁵.

In some embodiments, R⁴ is a cyclic group selected from a 3-6 membered saturated or partially unsaturated monocyclic carbocyclic ring, a 8-10 membered saturated or partially unsaturated bicyclic carbocyclic ring, phenyl, an 8-10 membered bicyclic aromatic carbocyclic ring, a 3-6 membered saturated or partially unsaturated monocyclic heterocyclic ring having 1-4 heteroatoms independently selected from nitrogen, oxygen, and sulfur, a 8-10 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵.

In some embodiments, R⁴ is a cyclic group selected from a 3-6 membered saturated or partially unsaturated monocyclic carbocyclic ring, a 8-10 membered saturated or partially unsaturated bicyclic carbocyclic ring, phenyl, an 8-10 membered bicyclic aromatic carbocyclic ring, a 3-6 membered saturated or partially unsaturated monocyclic heterocyclic ring having 2-4 heteroatoms independently selected from nitrogen, oxygen, and sulfur, a 8-10 membered saturated or partially unsaturated bicyclic heterocyclic ring having 2-4 heteroatoms independently selected from nitrogen, oxygen, and sulfur, a 5-6 membered monocyclic heteroaromatic ring having 2-4 heteroatoms independently selected from nitrogen, oxygen, and sulfur, and an 8-10 membered bicyclic heteroaromatic ring having 2-5 heteroatoms independently selected from nitrogen, oxygen, and sulfur, wherein R⁴ is substituted with p instances of R⁵.

In some embodiments, R⁴ is selected from those depicted in Table 1, below.

As defined generally above, R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence halo. In certain embodiments, R⁵ represents independently for each occurrence —OR. In certain embodiments, R⁵ represents independently for each occurrence —SR. In certain embodiments, R⁵ represents independently for each occurrence —NR₂. In certain embodiments, R⁵ represents independently for each occurrence —S(O)₂R. In certain embodiments, R⁵ represents independently for each occurrence —S(O)₂NR₂. In certain embodiments, R⁵ represents independently for each occurrence —S(O)R. In certain embodiments, R⁵ represents independently for each occurrence —S(O)NR₂. In certain embodiments, R⁵ represents independently for each occurrence —C(O)R. In certain embodiments, R⁵ represents independently for each occurrence —C(O)CR₃. In certain embodiments, R⁵ represents independently for each occurrence —C(O)OR. In certain embodiments, R⁵ represents independently for each occurrence —C(O)NR₂. In certain embodiments, R⁵ represents independently for each occurrence —C(O)N(R)OR. In certain embodiments, R⁵ represents independently for each occurrence —OC(O)R. In certain embodiments, R⁵ represents independently for each occurrence —OC(O)NR₂. In certain embodiments, R⁵ represents independently for each occurrence —O(CR₂)₃NR₂. In certain embodiments, R⁵ represents independently for each occurrence —N(R)C(O)OR. In certain embodiments, R⁵ represents independently for each occurrence —N(R)C(O)R. In certain embodiments, R⁵ represents independently for each occurrence —N(R)C(O)NR₂. In certain embodiments, R⁵ represents independently for each occurrence —N(R)C(NR)NR₂. In certain embodiments, R⁵ represents independently for each occurrence-NR(CR₂)₂OR. In certain embodiments, R⁵ represents independently for each occurrence —N(R)S(O)₂NR₂. In certain embodiments, R⁵ represents independently for each occurrence —N(R)S(O)₂R. In certain embodiments, R⁵ represents independently for each occurrence —CH₂R. In certain embodiments, R⁵ represents independently for each occurrence —CHR₂. In certain embodiments, R⁵ represents independently for each occurrence —CR₃. In certain embodiments, R⁵ represents independently for each occurrence —CH₂NR₂. In certain embodiments, R⁵ represents independently for each occurrence —CH₂NHCH₂CH₂OR. In certain embodiments, R⁵ represents independently for each occurrence —CH₂CH₂C(O)OR. In certain embodiments, R⁵ represents independently for each occurrence —CH₂C(O)NR₂. In certain embodiments, R⁵ represents independently for each occurrence —CH₂CH₂R, In certain embodiments, R⁵ represents independently for each occurrence —CH₂CH₂OR. In certain embodiments, R⁵ represents independently for each occurrence —NHCH₂CH₂OR. In certain embodiments, R⁵ represents independently for each occurrence —NHC(O)R. In certain embodiments, R⁵ represents independently for each occurrence —CH₂OR. In certain embodiments, R⁵ represents independently for each occurrence —CH₂NHC(O)OCR₃. In certain embodiments, R⁵ represents independently for each occurrence a C₁₋₃ alkyl group. In certain embodiments, R⁵ represents independently for each occurrence a C₁₋₃ haloalkyl group. In certain embodiments, R⁵ represents independently for each occurrence a C₁₋₃ heteroalkyl group having 1-2 heteroatoms independently selected from nitrogen, oxygen, and sulfur. In certain embodiments, R⁵ represents independently for each occurrence a 3-6 membered saturated or partially unsaturated monocyclic carbocyclic ring. In certain embodiments, R⁵ represents independently for each occurrence phenyl. In certain embodiments, R⁵ represents independently for each occurrence a 5-6 membered monocyclic heteroaromatic ring having 1-4 heteroatoms independently selected from nitrogen, oxygen, and sulfur. In certain embodiments, R⁵ represents independently for each occurrence a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence oxo, —OR, —OC(O)R, —OC(O)NR₂, or —O(CR₂)₃NR₂.

In certain embodiments, R⁵ represents independently for each occurrence —CN, —C(O)R, —C(O)CR₃, —C(O)OR, —C(O)NR₂, —C(O)N(R)OR, —CR₃, —CH₂NR₂, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In certain embodiments, R⁵ represents independently for each occurrence a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, or a C₁₋₃ haloalkyl group.

In certain embodiments, R⁵ represents independently for each occurrence halo, oxo, —CN, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ represents independently for each occurrence halo, oxo, a C₁₋₃ alkyl group, a C₁₋₃ haloalkyl group, or a C₁₋₃ heteroalkyl group having 1-2 heteroatoms independently selected from nitrogen, oxygen, and sulfur.

In certain embodiments, R⁵ is halo. In certain embodiments, R⁵ is —OR. In certain embodiments, R⁵ is —SR. In certain embodiments, R⁵ is —NR₂. In certain embodiments, R⁵ is —S(O)₂R. In certain embodiments, R⁵ is —S(O)₂NR₂. In certain embodiments, R⁵ is —S(O)R. In certain embodiments, R⁵ is —S(O)NR₂. In certain embodiments, R⁵ is —C(O)R. In certain embodiments, R⁵ is —C(O)CR₃. In certain embodiments, R⁵ is —C(O)OR. In certain embodiments, R⁵ is —C(O)NR₂. In certain embodiments, R⁵ is —C(O)N(R)OR. In certain embodiments, R⁵ is —OC(O)R. In certain embodiments, R⁵ is —OC(O)NR₂. In certain embodiments, R¹ is —O(CR₂)₃NR₂. In certain embodiments, R⁵ is —N(R)C(O)OR. In certain embodiments, R¹ is —N(R)C(O)R. In certain embodiments, R¹ is —N(R)C(O)NR₂. In certain embodiments, R⁵ is —N(R)C(NR)NR₂. In certain embodiments, R⁵ is-NR(CR₂)₂OR. In certain embodiments, R⁵ is —N(R)S(O)₂NR₂. In certain embodiments, R⁵ is —N(R)S(O)₂R. In certain embodiments, R¹ is —CH₂R. In certain embodiments, R⁵ is —CHR₂. In certain embodiments, R⁵ is —CR₃. In certain embodiments, R⁵ is —CH₂NR₂. In certain embodiments, R⁵ is —CH₂NHCH₂CH₂OR. In certain embodiments, R⁵ is —CH₂CH₂C(O)OR. In certain embodiments, R⁵ is —CH₂C(O)NR₂. In certain embodiments, R⁵ is —CH₂CH₂R, In certain embodiments, R⁵ is —CH₂CH₂OR. In certain embodiments, R⁵ is —NHCH₂CH₂OR. In certain embodiments, R⁵ is —NHC(O)R. In certain embodiments, R⁵ is —CH₂OR. In certain embodiments, R⁵ is —CH₂NHC(O)OCR₃. In certain embodiments, R⁵ isa C₁₋₃ alkyl group. In certain embodiments, R⁵ is a C₁₋₃ haloalkyl group. In certain embodiments, R⁵ is a C₁₋₃ heteroalkyl group having 1-2 heteroatoms independently selected from nitrogen, oxygen, and sulfur. In certain embodiments, R⁵ is a 3-6 membered saturated or partially unsaturated monocyclic carbocyclic ring. In certain embodiments, R⁵ is phenyl. In certain embodiments, R⁵ is a 5-6 membered monocyclic heteroaromatic ring having 1-4 heteroatoms independently selected from nitrogen, oxygen, and sulfur. In certain embodiments, R⁵ is a 3-6 membered saturated or partially unsaturated monocyclic heterocyclic ring having 1-4 heteroatoms independently selected from nitrogen, oxygen, and sulfur.

In certain embodiments, R⁵ is oxo, —OR, —OC(O)R, —OC(O)NR₂, or —O(CR₂)₃NR₂.

In certain embodiments, R⁵ is —CN, —C(O)R, —C(O)CR₃, —C(O)OR, —C(O)NR₂, —C(O)N(R)OR, —CR₃, —CH₂NR₂—, —CH₂NHCH₂CH₂OR, —CH₂CH₂C(O)OR, —CH₂C(O)NR₂, —CH₂CH₂R, —CH₂CH₂OR, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ is —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —NHCH₂CH₂OR, or —NHC(O)R.

In certain embodiments, R⁵ is a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ is halo, oxo, —CN, —OR, —SR, —NR₂, —S(O)₂R. —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂—, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, or a C₁₋₃ haloalkyl group.

In certain embodiments, R⁵ is halo, oxo, —CN, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur.

In certain embodiments, R⁵ is halo, oxo, a C₁₋₃ alkyl group, a C₁₋₃ haloalkyl group, or a C₁₋₃ heteroalkyl group having 1-2 heteroatoms independently selected from nitrogen, oxygen, and sulfur.

In some embodiments, R⁵ is selected from those depicted in Table 1, below.

As defined generally above, R represents independently for each occurrence hydrogen, hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence halo. In certain embodiments, R represents independently for each occurrence optionally substituted C₁₋₆ aliphatic. In certain embodiments, R represents independently for each occurrence optionally substituted phenyl. In certain embodiments, R represents independently for each occurrence optionally substituted 3-7 membered saturated or partially unsaturated monocyclic heterocyclic ring having 1-4 heteroatoms independently selected from nitrogen, oxygen, and sulfur. In certain embodiments, R represents independently for each occurrence optionally substituted 5-6 membered monocyclic heteroaryl ring having 1-4 heteroatoms independently selected from nitrogen, oxygen, and sulfur. In certain embodiments, R represents independently for each occurrence optionally substituted 3-7 membered saturated or partially unsaturated monocyclic carbocyclic ring.

In certain embodiments, R represents independently for each occurrence halo. In certain embodiments, R represents independently for each occurrence C₁₋₆ aliphatic. In certain embodiments, R represents independently for each occurrence phenyl. In certain embodiments, R represents independently for each occurrence 3-7 membered saturated or partially unsaturated monocyclic heterocyclic ring having 1-4 heteroatoms independently selected from nitrogen, oxygen, and sulfur. In certain embodiments, R represents independently for each occurrence 5-6 membered monocyclic heteroaryl ring having 1-4 heteroatoms independently selected from nitrogen, oxygen, and sulfur. In certain embodiments, R represents independently for each occurrence 3-7 membered saturated or partially unsaturated monocyclic carbocyclic ring.

In certain embodiments, R represents independently for each occurrence hydrogen, hydroxyl, halo, or a group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence hydrogen, hydroxyl, halo, or optionally substituted C₁₋₆ aliphatic.

In certain embodiments, R represents independently for each occurrence an optionally substituted group selected from phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R represents independently for each occurrence hydrogen, hydroxyl, halo, or an optionally substituted group selected from C₁₋₃ aliphatic, phenyl, a 3-6 membered saturated or partially unsaturated monocyclic heterocyclic ring having 1-3 heteroatoms independently selected from nitrogen, oxygen, and sulfur, a 5-6 membered monocyclic heteroaryl ring having 1-3 heteroatoms independently selected from nitrogen, oxygen, and sulfur, and a 3-6 membered saturated or partially unsaturated monocyclic carbocyclic ring.

In certain embodiments, R represents independently for each occurrence hydrogen, hydroxyl, Cl, F, —CH₂OH, —CH₂CH₂OH, methyl, ethyl, i-propyl, n-propyl, cyclopropyl, or phenyl.

In certain embodiments, R is halo. In certain embodiments, R is optionally substituted C₁₋₆ aliphatic. In certain embodiments, R is optionally substituted phenyl. In certain embodiments, R is optionally substituted 3-7 membered saturated or partially unsaturated monocyclic heterocyclic ring having 1-4 heteroatoms independently selected from nitrogen, oxygen, and sulfur. In certain embodiments, R is optionally substituted 5-6 membered monocyclic heteroaryl ring having 1-4 heteroatoms independently selected from nitrogen, oxygen, and sulfur. In certain embodiments, R is optionally substituted 3-7 membered saturated or partially unsaturated monocyclic carbocyclic ring.

In certain embodiments, R is halo. In certain embodiments, R is C₁₋₆ aliphatic. In certain embodiments, R is phenyl. In certain embodiments, R is 3-7 membered saturated or partially unsaturated monocyclic heterocyclic ring having 1-4 heteroatoms independently selected from nitrogen, oxygen, and sulfur. In certain embodiments, R is 5-6 membered monocyclic heteroaryl ring having 1-4 heteroatoms independently selected from nitrogen, oxygen, and sulfur. In certain embodiments, R is 3-7 membered saturated or partially unsaturated monocyclic carbocyclic ring.

In certain embodiments, R is hydrogen, hydroxyl, halo, or a group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R is hydrogen, hydroxyl, halo, or optionally substituted C₁₋₆ aliphatic.

In certain embodiments, R is an optionally substituted group selected from phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R is hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring.

In certain embodiments, R is hydrogen, hydroxyl, halo, or an optionally substituted group selected from C₁₋₃ aliphatic, phenyl, a 3-6 membered saturated or partially unsaturated monocyclic heterocyclic ring having 1-3 heteroatoms independently selected from nitrogen, oxygen, and sulfur, a 5-6 membered monocyclic heteroaryl ring having 1-3 heteroatoms independently selected from nitrogen, oxygen, and sulfur, and a 3-6 membered saturated or partially unsaturated monocyclic carbocyclic ring.

In certain embodiments, R is hydrogen, hydroxyl, Cl, F, —CH₂OH, —CH₂CH₂OH, methyl, ethyl, i-propyl, n-propyl, cyclopropyl, or phenyl.

In some embodiments, R is selected from those depicted in Table 1, below.

As defined generally above, n is 0, 1, 2, or 3. In certain embodiments, n is 1, 2, or 3. In certain embodiments, n is 0, 2, or 3. In certain embodiments, n is 0, 1, or 3. In certain embodiments, n is 0, 1, or 2. In certain embodiments, n is 2 or 3. In certain embodiments, n is 0 or 1. In certain embodiments, n is 0 or 3. In certain embodiments, n is 0 or 2. In certain embodiments, n is 1 or 2. In certain embodiments, n is 1 or 3. In certain embodiments, n is 0. In certain embodiments, n is 1. In certain embodiments, n is 2. In certain embodiments, n is 3.

As defined generally above, p is 0, 1, 2, 3 or 4. In certain embodiments, p is 1, 2, 3 or 4. In certain embodiments, p is 0, 2, 3 or 4. In certain embodiments, p is 0, 1, 3 or 4. In certain embodiments, p is 0, 1, 2, or 4. In certain embodiments, p is 0, 1, 2, or 3. In certain embodiments, p is 2, 3 or 4. In certain embodiments, p is 0, 3 or 4. In certain embodiments, p is 0, 1, or 4. In certain embodiments, p is 0, 1, or 2. In certain embodiments, p is 1, 3 or 4. In certain embodiments, p is 1, 2, or 4. In certain embodiments, p is 1, 2, or 3. In certain embodiments, p is 0, 2 or 4. In certain embodiments, p is 0, 2 or 3. In certain embodiments, p is 0, 1 or 3. In certain embodiments, p is 3 or 4. In certain embodiments, p is 0 or 4. In certain embodiments, p is 0 or 1. In certain embodiments, p is 1 or 4. In certain embodiments, p is 1 or 2. In certain embodiments, p is 0 or 2. In certain embodiments, p is 0 or 3. In certain embodiments, p is 1 or 3. In certain embodiments, p is 2 or 3. In certain embodiments, p is 2 or 4. In certain embodiments, p is 0. In certain embodiments, p is 1. In certain embodiments, p is 2. In certain embodiments, p is 3. In certain embodiments, p is 4.

### II. Exemplary Compounds

In certain embodiments, the compound is a compound in Table 1, or a pharmaceutically acceptable salt thereof. In certain embodiments, the compound is a compound in Table 1.

TABLE 1
Exemplary Compounds

| Compound | |
|---|---|
| No. | Structure |
| I-1 | 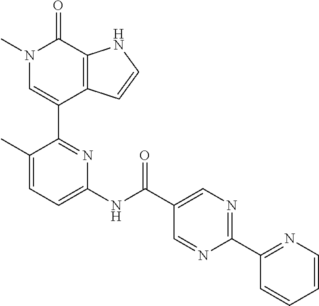 [SMILES: Cc1ccc(NC(=O)c2cnc(-c3ccccn3)nc2)nc1-c1cn(C)c(=O)c2[nH]ccc12] |
| I-2 | 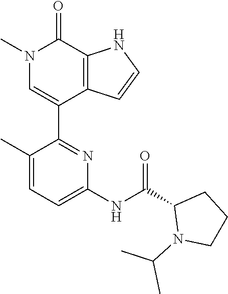 [SMILES: Cc1ccc(NC(=O)[C@@H]2CCCN2C(C)C)nc1-c1cn(C)c(=O)c2[nH]ccc12] |
| I-3 | 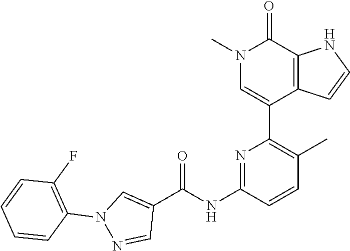 [SMILES: Cc1ccc(NC(=O)c2cnn(-c3ccccc3F)c2)nc1-c1cn(C)c(=O)c2[nH]ccc12] |
| I-4 | 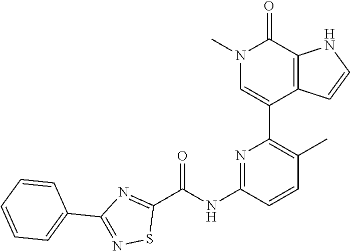 [SMILES: Cc1ccc(NC(=O)c2nc(-c3ccccc3)ns2)nc1-c1cn(C)c(=O)c2[nH]ccc12] |
| I-5 | 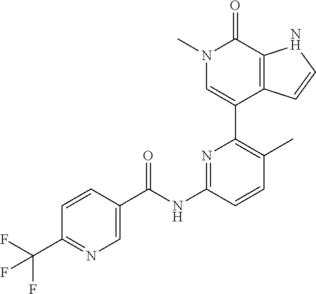 [SMILES: Cc1ccc(NC(=O)c2ccc(C(F)(F)F)nc2)nc1-c1cn(C)c(=O)c2[nH]ccc12] |
| I-6 | 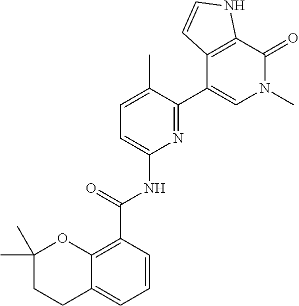 |
| I-7 | 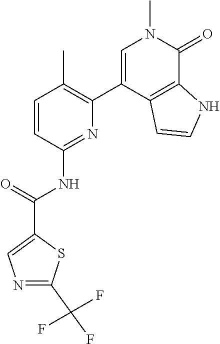 [SMILES: Cc1ccc(NC(=O)c2cnc(C(F)(F)F)s2)nc1-c1cn(C)c(=O)c2[nH]ccc12] |
| I-8 | 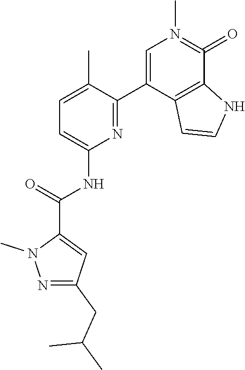 |
| I-9 | 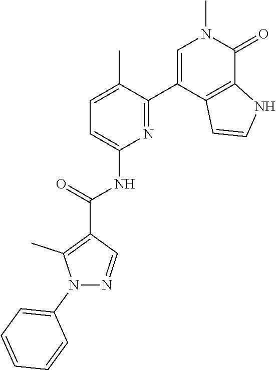 [SMILES: Cc1ccc(NC(=O)c2cnn(-c3ccccc3)c2C)nc1-c1cn(C)c(=O)c2[nH]ccc12] |
| I-10 | 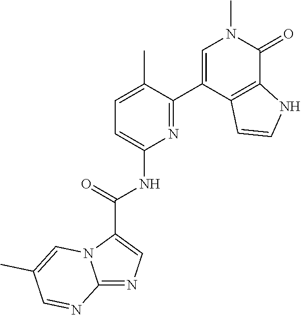 [SMILES: Cc1cnc2ncc(C(=O)Nc3ccc(C)c(-c4cn(C)c(=O)c5[nH]ccc45)n3)n2c1] |
| I-11 | 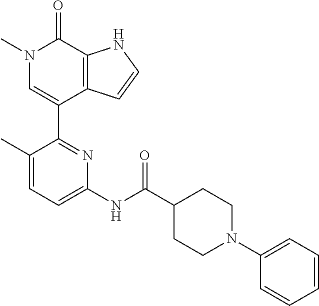 [SMILES: Cc1ccc(NC(=O)C2CCN(c3ccccc3)CC2)nc1-c1cn(C)c(=O)c2[nH]ccc12] |
| I-12 | 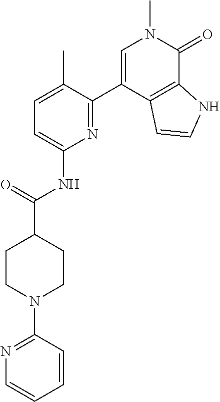 [SMILES: Cc1ccc(NC(=O)C2CCN(c3ccccn3)CC2)nc1-c1cn(C)c(=O)c2[nH]ccc12] |
| I-13 | 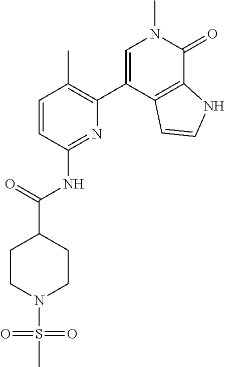 [SMILES: Cc1ccc(NC(=O)C2CCN(S(C)(=O)=O)CC2)nc1-c1cn(C)c(=O)c2[nH]ccc12] |
| I-14 | 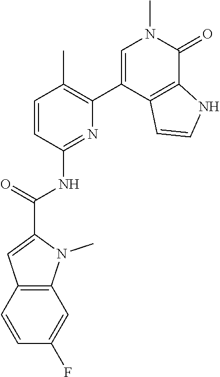 [SMILES: Cc1ccc(NC(=O)c2cc3ccc(F)cc3n2C)nc1-c1cn(C)c(=O)c2[nH]ccc12] |
| I-15 | 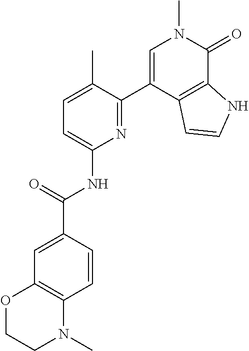 [SMILES: Cc1ccc(NC(=O)c2ccc3c(c2)OCCN3C)nc1-c1cn(C)c(=O)c2[nH]ccc12] |
| I-16 | 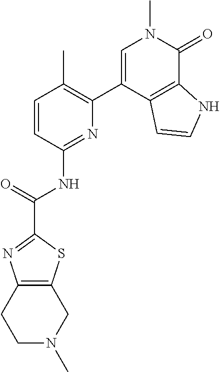 [SMILES: Cc1ccc(NC(=O)c2nc3c(s2)CN(C)CC3)nc1-c1cn(C)c(=O)c2[nH]ccc12] |
| I-17 | 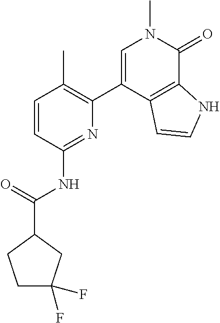 [SMILES: Cc1ccc(NC(=O)C2CCC(F)(F)C2)nc1-c1cn(C)c(=O)c2[nH]ccc12] |
| I-18 | 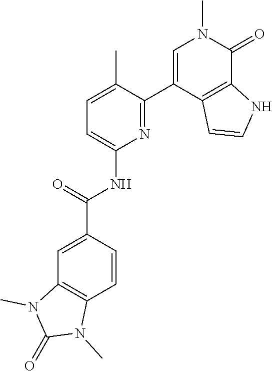 [SMILES: Cc1ccc(NC(=O)c2ccc3c(c2)n(C)c(=O)n3C)nc1-c1cn(C)c(=O)c2[nH]ccc12] |
| I-19 | 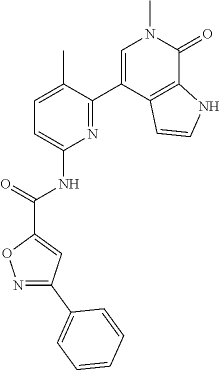 [SMILES: Cc1ccc(NC(=O)c2cc(-c3ccccc3)no2)nc1-c1cn(C)c(=O)c2[nH]ccc12] |
| I-20 | 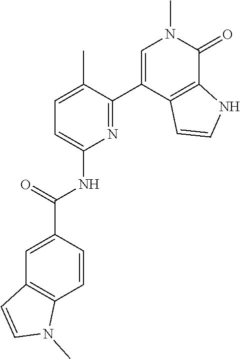 [SMILES: Cc1ccc(NC(=O)c2ccc3c(ccn3C)c2)nc1-c1cn(C)c(=O)c2[nH]ccc12] |
| I-21 | 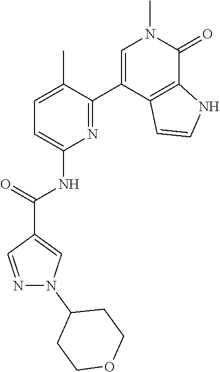 [SMILES: Cc1ccc(NC(=O)c2cnn(C3CCOCC3)c2)nc1-c1cn(C)c(=O)c2[nH]ccc12] |
| I-22 | 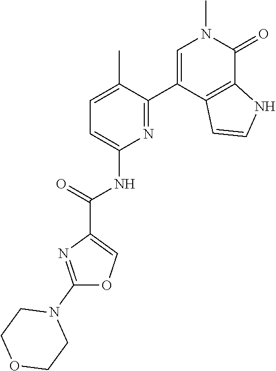 [SMILES: Cc1ccc(NC(=O)c2coc(N3CCOCC3)n2)nc1-c1cn(C)c(=O)c2[nH]ccc12] |
| I-23 | 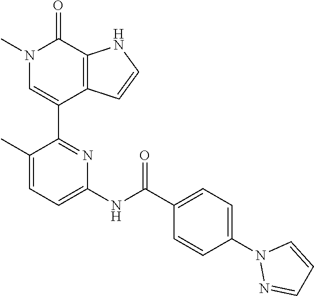 [SMILES: Cc1ccc(NC(=O)c2ccc(-n3cccn3)cc2)nc1-c1cn(C)c(=O)c2[nH]ccc12] |
| I-24 | 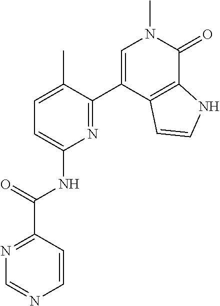 [SMILES: Cc1ccc(NC(=O)c2ccncn2)nc1-c1cn(C)c(=O)c2[nH]ccc12] |
| I-25 | 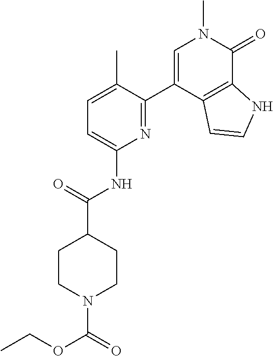 [SMILES: CCOC(=O)N1CCC(C(=O)Nc2ccc(C)c(-c3cn(C)c(=O)c4[nH]ccc34)n2)CC1] |
| I-26 | 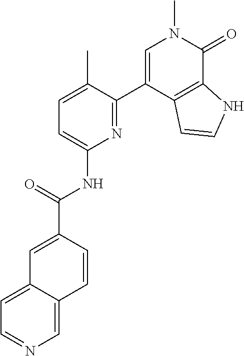 [SMILES: Cc1ccc(NC(=O)c2ccc3cnccc3c2)nc1-c1cn(C)c(=O)c2[nH]ccc12] |
| I-27 | 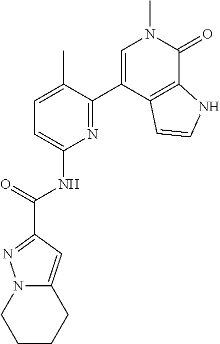 [SMILES: Cc1ccc(NC(=O)c2cc3n(n2)CCCC3)nc1-c1cn(C)c(=O)c2[nH]ccc12] |
| I-28 | 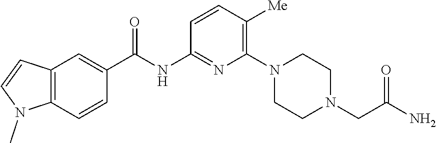 [SMILES: Cc1ccc(NC(=O)c2ccc3c(ccn3C)c2)nc1N1CCN(CC(N)=O)CC1] |
| I-29 | 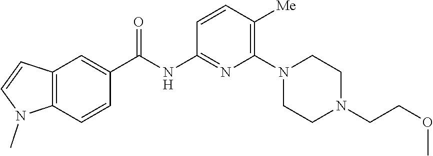 [SMILES: COCCN1CCN(c2nc(NC(=O)c3ccc4c(ccn4C)c3)ccc2C)CC1] |
| I-30 | 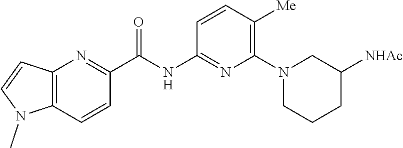 [SMILES: CC(=O)NC1CCCN(c2nc(NC(=O)c3ccc4c(ccn4C)n3)ccc2C)C1] |
| I-31 | 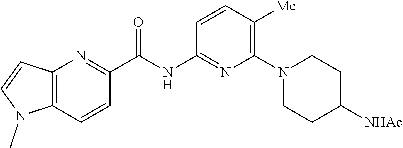 [SMILES: CC(=O)NC1CCN(c2nc(NC(=O)c3ccc4c(ccn4C)n3)ccc2C)CC1] |
| I-32 | 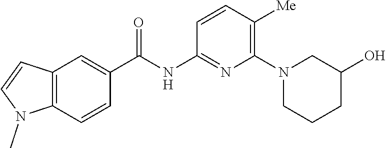 [SMILES: Cc1ccc(NC(=O)c2ccc3c(ccn3C)c2)nc1N1CCCC(O)C1] |
| I-33 | 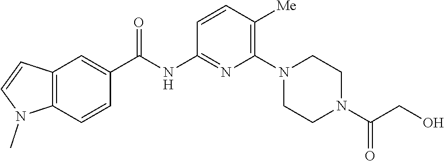 [SMILES: Cc1ccc(NC(=O)c2ccc3c(ccn3C)c2)nc1N1CCN(C(=O)CO)CC1] |
| I-34 | 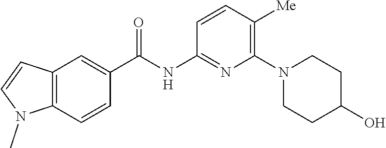 [SMILES: Cc1ccc(NC(=O)c2ccc3c(ccn3C)c2)nc1N1CCC(O)CC1] |
| I-35 | 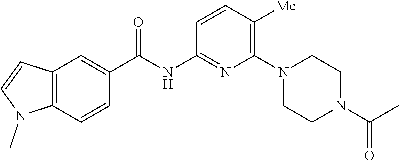 [SMILES: CC(=O)N1CCN(c2nc(NC(=O)c3ccc4c(ccn4C)c3)ccc2C)CC1] |
| I-36 | 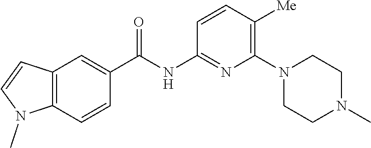 [SMILES: Cc1ccc(NC(=O)c2ccc3c(ccn3C)c2)nc1N1CCN(C)CC1] |
| I-37 | 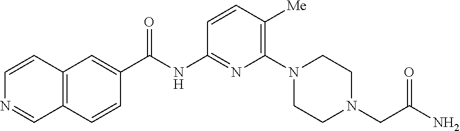 [SMILES: Cc1ccc(NC(=O)c2ccc3cnccc3c2)nc1N1CCN(CC(N)=O)CC1] |
| I-38 | 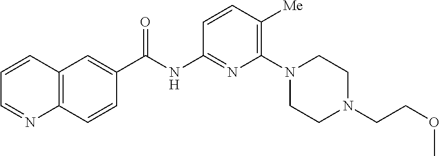 [SMILES: COCCN1CCN(c2nc(NC(=O)c3ccc4ncccc4c3)ccc2C)CC1] |
| I-39 | 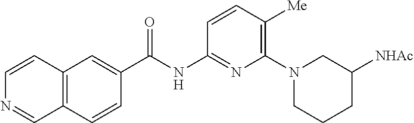 [SMILES: CC(=O)NC1CCCN(c2nc(NC(=O)c3ccc4cnccc4c3)ccc2C)C1] |
| I-40 | 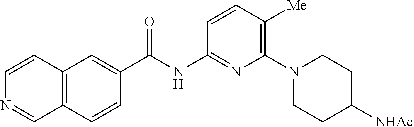 [SMILES: CC(=O)NC1CCN(c2nc(NC(=O)c3ccc4cnccc4c3)ccc2C)CC1] |
| I-41 | 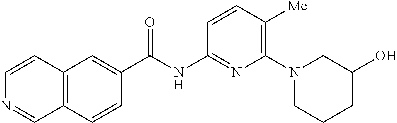 [SMILES: Cc1ccc(NC(=O)c2ccc3cnccc3c2)nc1N1CCCC(O)C1] |
| I-42 | 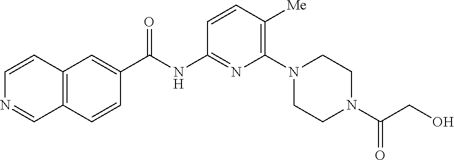 [SMILES: Cc1ccc(NC(=O)c2ccc3cnccc3c2)nc1N1CCN(C(=O)CO)CC1] |
| I-43 | 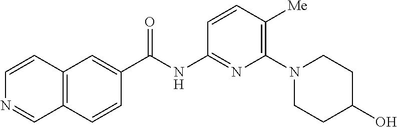 [SMILES: Cc1ccc(NC(=O)c2ccc3cnccc3c2)nc1N1CCC(O)CC1] |
| I-44 | 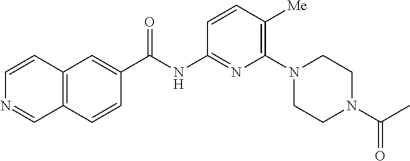 [SMILES: CC(=O)N1CCN(c2nc(NC(=O)c3ccc4cnccc4c3)ccc2C)CC1] |
| I-45 | 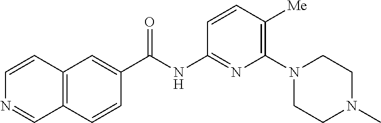 [SMILES: Cc1ccc(NC(=O)c2ccc3cnccc3c2)nc1N1CCN(C)CC1] |
| I-46 | 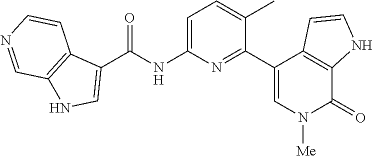 [SMILES: Cc1ccc(NC(=O)c2c[nH]c3cnccc23)nc1-c1cn(C)c(=O)c2[nH]ccc12] |
| I-47 | 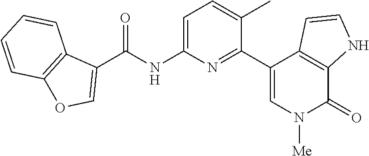 [SMILES: Cc1ccc(NC(=O)c2coc3ccccc23)nc1-c1cn(C)c(=O)c2[nH]ccc12] |
| I-48 | 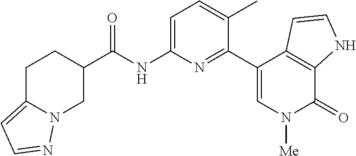 [SMILES: Cc1ccc(NC(=O)C2CCc3ccnn3C2)nc1-c1cn(C)c(=O)c2[nH]ccc12] |
| I-49 | 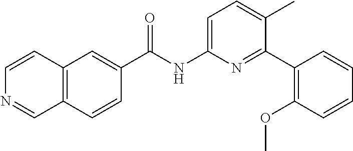 [SMILES: COc1ccccc1-c1nc(NC(=O)c2ccc3cnccc3c2)ccc1C] |
| I-50 | 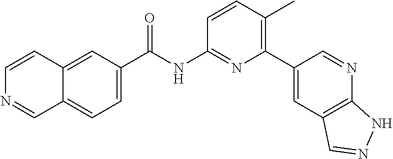 [SMILES: Cc1ccc(NC(=O)c2ccc3cnccc3c2)nc1-c1cnc2[nH]ncc2c1] |
| I-51 | 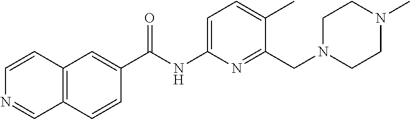 [SMILES: Cc1ccc(NC(=O)c2ccc3cnccc3c2)nc1CN1CCN(C)CC1] |
| I-52 | 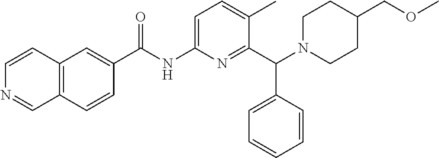 [SMILES: COCC1CCN(C(c2ccccc2)c2nc(NC(=O)c3ccc4cnccc4c3)ccc2C)CC1] |
| I-53 | 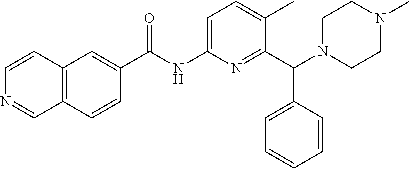 [SMILES: Cc1ccc(NC(=O)c2ccc3cnccc3c2)nc1C(c1ccccc1)N1CCN(C)CC1] |
| I-54 | 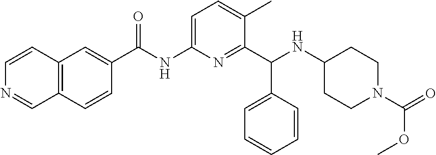 [SMILES: COC(=O)N1CCC(NC(c2ccccc2)c2nc(NC(=O)c3ccc4cnccc4c3)ccc2C)CC1] |
| I-55 | 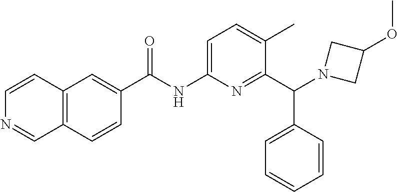 [SMILES: COC1CN(C(c2ccccc2)c2nc(NC(=O)c3ccc4cnccc4c3)ccc2C)C1] |
| I-56 | 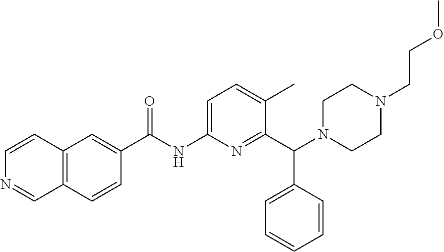 [SMILES: COCCN1CCN(C(c2ccccc2)c2nc(NC(=O)c3ccc4cnccc4c3)ccc2C)CC1] |
| I-57 | 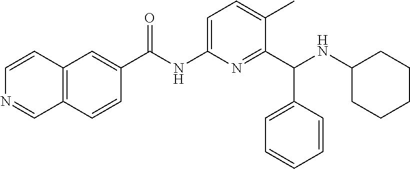 [SMILES: Cc1ccc(NC(=O)c2ccc3cnccc3c2)nc1C(NC1CCCCC1)c1ccccc1] |
| I-58 | 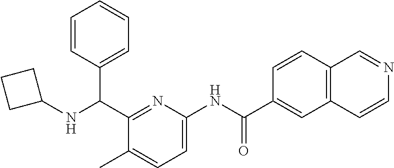 [SMILES: Cc1ccc(NC(=O)c2ccc3cnccc3c2)nc1C(NC1CCC1)c1ccccc1] |
| I-59 | 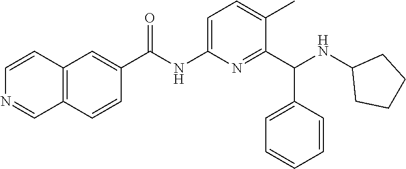 [SMILES: Cc1ccc(NC(=O)c2ccc3cnccc3c2)nc1C(NC1CCCC1)c1ccccc1] |
| I-60 | 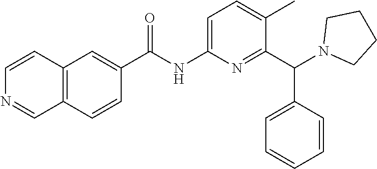 [SMILES: Cc1ccc(NC(=O)c2ccc3cnccc3c2)nc1C(c1ccccc1)N1CCCC1] |
| I-61 | 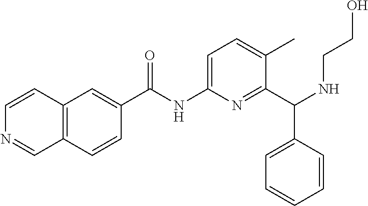 [SMILES: Cc1ccc(NC(=O)c2ccc3cnccc3c2)nc1C(NCCO)c1ccccc1] |
| I-62 | 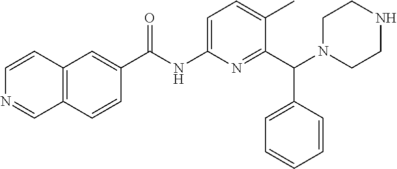 [SMILES: Cc1ccc(NC(=O)c2ccc3cnccc3c2)nc1C(c1ccccc1)N1CCNCC1] |
| I-63 | 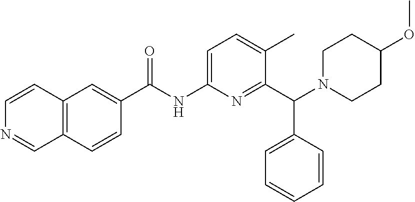 [SMILES: COC1CCN(C(c2ccccc2)c2nc(NC(=O)c3ccc4cnccc4c3)ccc2C)CC1] |
| I-64 | 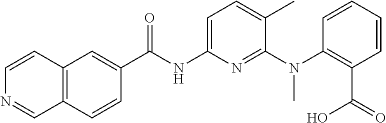 [SMILES: Cc1ccc(NC(=O)c2ccc3cnccc3c2)nc1N(C)c1ccccc1C(=O)O] |
| I-65 | 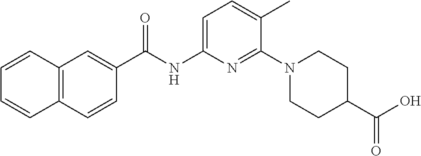 [SMILES: Cc1ccc(NC(=O)c2ccc3ccccc3c2)nc1N1CCC(C(=O)O)CC1] |
| I-66 | 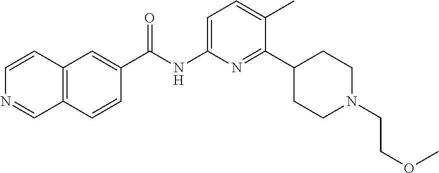 [SMILES: COCCN1CCC(c2nc(NC(=O)c3ccc4cnccc4c3)ccc2C)CC1] |
| I-67 | 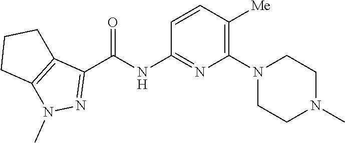 [SMILES: Cc1ccc(NC(=O)c2nn(C)c3c2CCC3)nc1N1CCN(C)CC1] |
| I-68 | 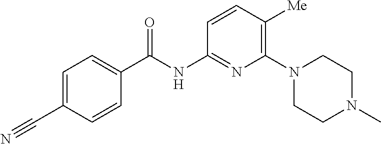 [SMILES: Cc1ccc(NC(=O)c2ccc(C#N)cc2)nc1N1CCN(C)CC1] |
| I-69 | 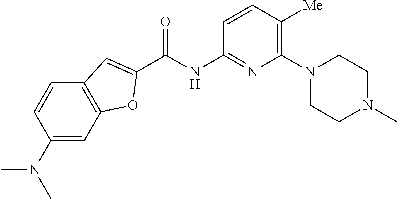 |
| I-70 | 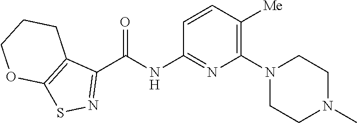 [SMILES: Cc1ccc(NC(=O)c2nsc3c2CCCO3)nc1N1CCN(C)CC1] |
| I-71 | 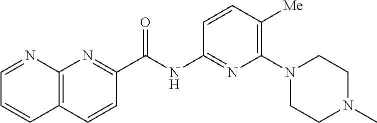 [SMILES: Cc1ccc(NC(=O)c2ccc3cccnc3n2)nc1N1CCN(C)CC1] |
| I-72 | 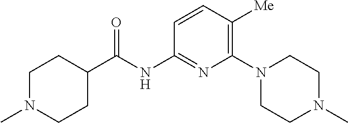 |
| I-73 | 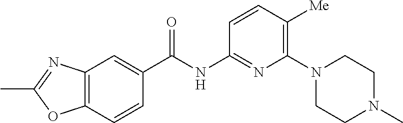 |
| I-74 | 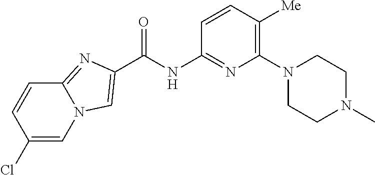 [SMILES: Cc1ccc(NC(=O)c2cn3cc(Cl)ccc3n2)nc1N1CCN(C)CC1] |
| I-75 | 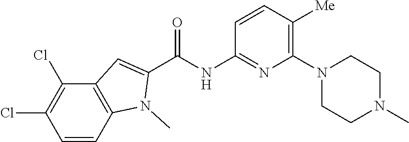 [SMILES: Cc1ccc(NC(=O)c2cc3c(Cl)c(Cl)ccc3n2C)nc1N1CCN(C)CC1] |
| I-76 | 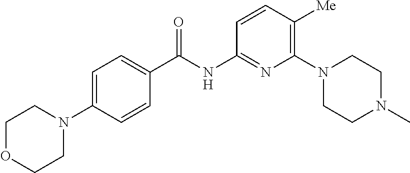 [SMILES: Cc1ccc(NC(=O)c2ccc(N3CCOCC3)cc2)nc1N1CCN(C)CC1] |
| I-77 | 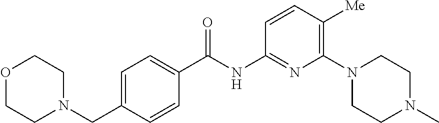 [SMILES: Cc1ccc(NC(=O)c2ccc(CN3CCOCC3)cc2)nc1N1CCN(C)CC1] |
| I-78 | 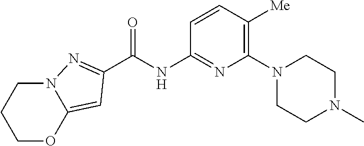 [SMILES: Cc1ccc(NC(=O)c2cc3n(n2)CCCO3)nc1N1CCN(C)CC1] |
| I-79 | 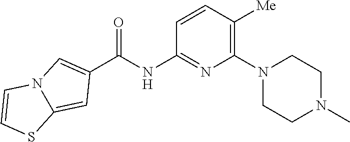 [SMILES: Cc1ccc(NC(=O)c2cc3sccn3c2)nc1N1CCN(C)CC1] |
| I-80 | 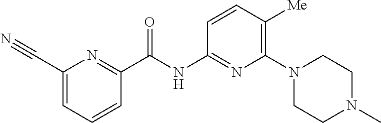 [SMILES: Cc1ccc(NC(=O)c2cccc(C#N)n2)nc1N1CCN(C)CC1] |
| I-81 | 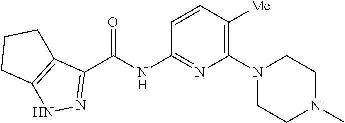 [SMILES: Cc1ccc(NC(=O)c2n[nH]c3c2CCC3)nc1N1CCN(C)CC1] |
| I-82 | 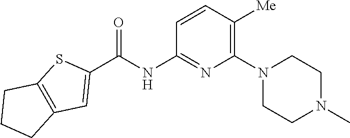 [SMILES: Cc1ccc(NC(=O)c2cc3c(s2)CCC3)nc1N1CCN(C)CC1] |
| I-83 | 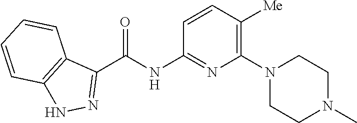 [SMILES: Cc1ccc(NC(=O)c2n[nH]c3ccccc23)nc1N1CCN(C)CC1] |
| I-84 | 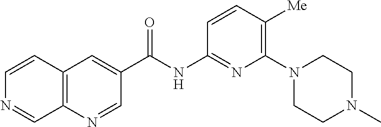 [SMILES: Cc1ccc(NC(=O)c2cnc3cnccc3c2)nc1N1CCN(C)CC1] |
| I-85 | 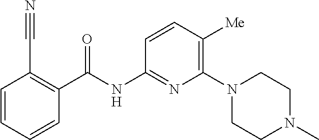 [SMILES: Cc1ccc(NC(=O)c2ccccc2C#N)nc1N1CCN(C)CC1] |
| I-86 | 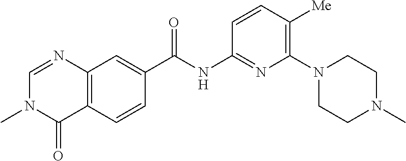 |
| I-87 | 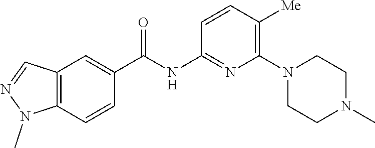 [SMILES: Cc1ccc(NC(=O)c2ccc3c(cnn3C)c2)nc1N1CCN(C)CC1] |
| I-88 | 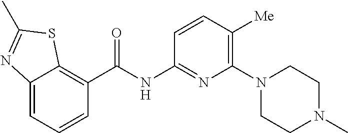 |
| I-89 | 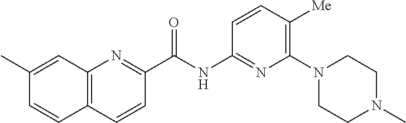 |
| I-90 | 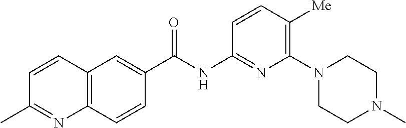 |
| I-91 | 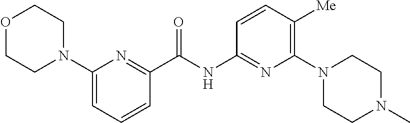 [SMILES: Cc1ccc(NC(=O)c2cccc(N3CCOCC3)n2)nc1N1CCN(C)CC1] |
| I-92 | 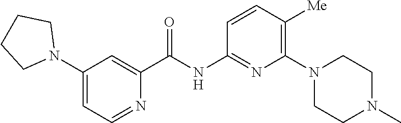 [SMILES: Cc1ccc(NC(=O)c2cc(N3CCCC3)ccn2)nc1N1CCN(C)CC1] |
| I-93 | 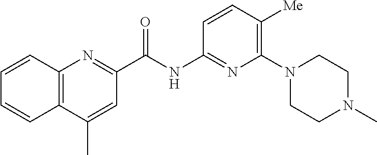 [SMILES: Cc1ccc(NC(=O)c2cc(C)c3ccccc3n2)nc1N1CCN(C)CC1] |
| I-94 | 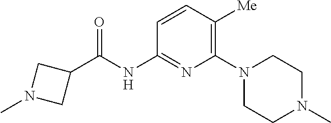 |
| I-95 | 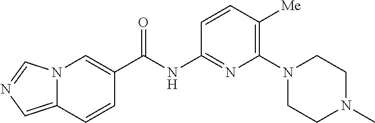 [SMILES: Cc1ccc(NC(=O)c2ccc3cncn3c2)nc1N1CCN(C)CC1] |
| I-96 | 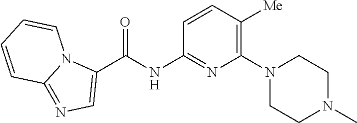 [SMILES: Cc1ccc(NC(=O)c2cnc3ccccn23)nc1N1CCN(C)CC1] |
| I-97 | 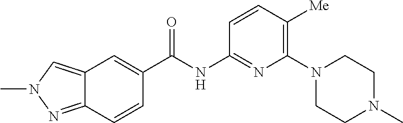 |
| I-98 | 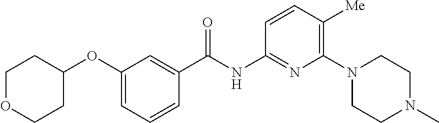 [SMILES: Cc1ccc(NC(=O)c2cccc(OC3CCOCC3)c2)nc1N1CCN(C)CC1] |
| I-99 | 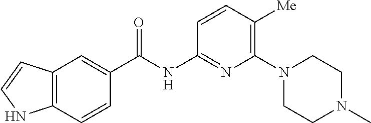 [SMILES: Cc1ccc(NC(=O)c2ccc3[nH]ccc3c2)nc1N1CCN(C)CC1] |
| I-100 | 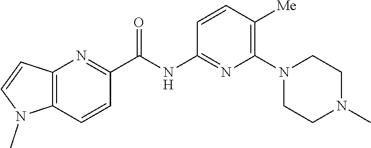 [SMILES: Cc1ccc(NC(=O)c2ccc3c(ccn3C)n2)nc1N1CCN(C)CC1] |
| I-101 | 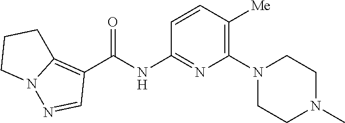 [SMILES: Cc1ccc(NC(=O)c2cnn3c2CCC3)nc1N1CCN(C)CC1] |
| I-102 | 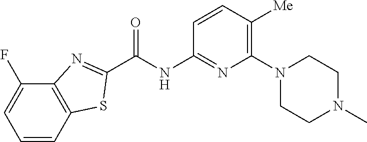 [SMILES: Cc1ccc(NC(=O)c2nc3c(F)cccc3s2)nc1N1CCN(C)CC1] |
| I-103 | 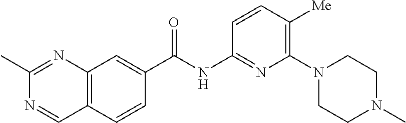 |
| I-104 | 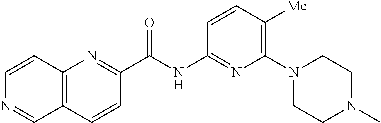 [SMILES: Cc1ccc(NC(=O)c2ccc3cnccc3n2)nc1N1CCN(C)CC1] |
| I-105 | 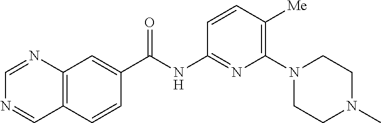 [SMILES: Cc1ccc(NC(=O)c2ccc3cncnc3c2)nc1N1CCN(C)CC1] |
| I-106 | 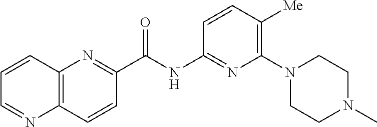 [SMILES: Cc1ccc(NC(=O)c2ccc3ncccc3n2)nc1N1CCN(C)CC1] |
| I-107 | 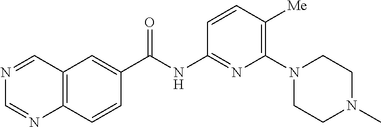 [SMILES: Cc1ccc(NC(=O)c2ccc3ncncc3c2)nc1N1CCN(C)CC1] |
| I-108 | 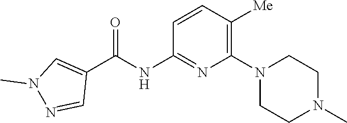 |
| I-109 | 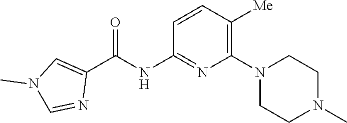 |
| I-110 | 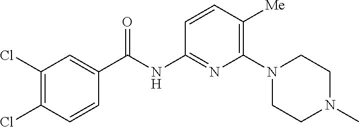 [SMILES: Cc1ccc(NC(=O)c2ccc(Cl)c(Cl)c2)nc1N1CCN(C)CC1] |
| I-111 | 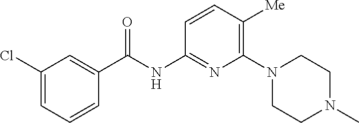 [SMILES: Cc1ccc(NC(=O)c2cccc(Cl)c2)nc1N1CCN(C)CC1] |
| I-112 | 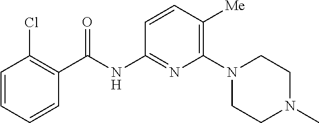 [SMILES: Cc1ccc(NC(=O)c2ccccc2Cl)nc1N1CCN(C)CC1] |
| I-113 | 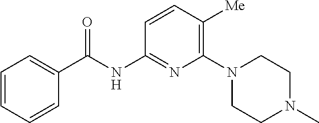 [SMILES: Cc1ccc(NC(=O)c2ccccc2)nc1N1CCN(C)CC1] |
| I-114 | 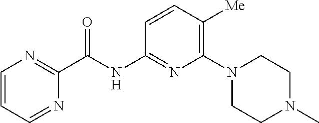 [SMILES: Cc1ccc(NC(=O)c2ncccn2)nc1N1CCN(C)CC1] |
| I-115 | 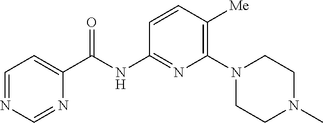 [SMILES: Cc1ccc(NC(=O)c2ccncn2)nc1N1CCN(C)CC1] |
| I-116 | 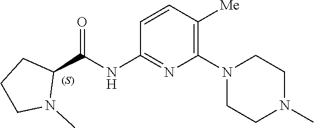 |
| I-117 | 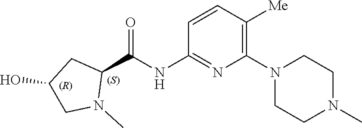 |
| I-118 | 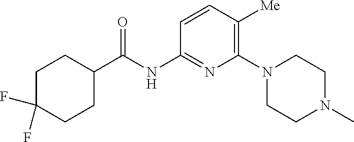 [SMILES: Cc1ccc(NC(=O)C2CCC(F)(F)CC2)nc1N1CCN(C)CC1] |
| I-119 | 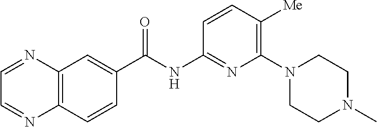 [SMILES: Cc1ccc(NC(=O)c2ccc3nccnc3c2)nc1N1CCN(C)CC1] |
| I-120 | 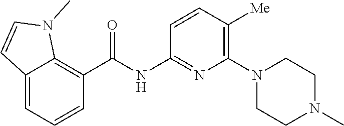 [SMILES: Cc1ccc(NC(=O)c2cccc3ccn(C)c23)nc1N1CCN(C)CC1] |
| I-121 | 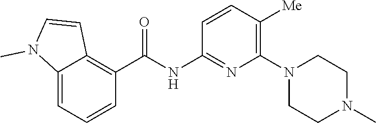 |
| I-122 | 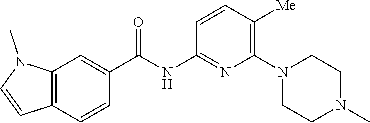 [SMILES: Cc1ccc(NC(=O)c2ccc3ccn(C)c3c2)nc1N1CCN(C)CC1] |
| I-123 | 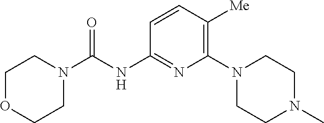 [SMILES: Cc1ccc(NC(=O)N2CCOCC2)nc1N1CCN(C)CC1] |
| I-124 | 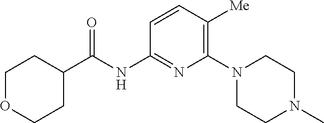 [SMILES: Cc1ccc(NC(=O)C2CCOCC2)nc1N1CCN(C)CC1] |
| I-125 | 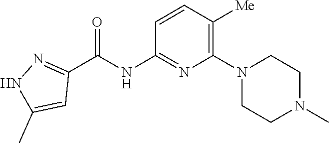 [SMILES: Cc1cc(C(=O)Nc2ccc(C)c(N3CCN(C)CC3)n2)n[nH]1] |
| I-126 | 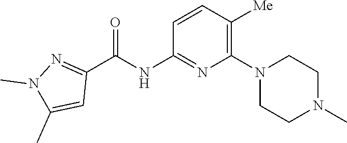 |
| I-127 | 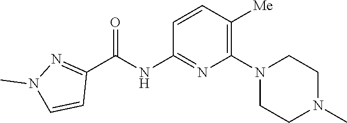 |
| I-128 | 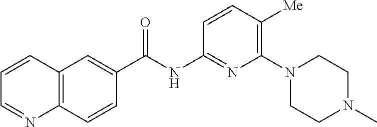 [SMILES: Cc1ccc(NC(=O)c2ccc3ncccc3c2)nc1N1CCN(C)CC1] |
| I-129 | 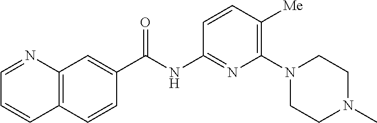 [SMILES: Cc1ccc(NC(=O)c2ccc3cccnc3c2)nc1N1CCN(C)CC1] |
| I-130 | 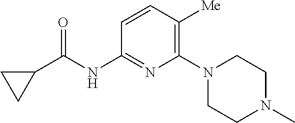 [SMILES: Cc1ccc(NC(=O)C2CC2)nc1N1CCN(C)CC1] |
| I-131 | 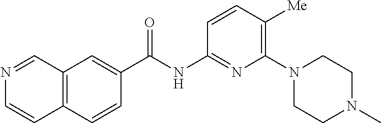 [SMILES: Cc1ccc(NC(=O)c2ccc3ccncc3c2)nc1N1CCN(C)CC1] |
| I-132 | 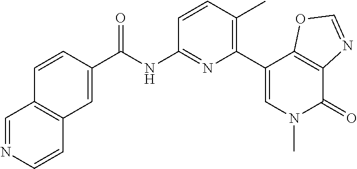 [SMILES: Cc1ccc(NC(=O)c2ccc3cnccc3c2)nc1-c1cn(C)c(=O)c2ncoc12] |
| I-133 | 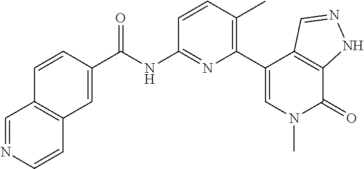 [SMILES: Cc1ccc(NC(=O)c2ccc3cnccc3c2)nc1-c1cn(C)c(=O)c2[nH]ncc12] |
| I-134 | 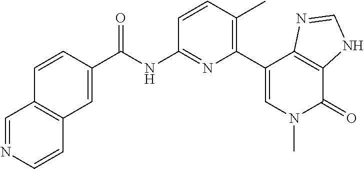 [SMILES: Cc1ccc(NC(=O)c2ccc3cnccc3c2)nc1-c1cn(C)c(=O)c2[nH]cnc12] |
| I-135 | 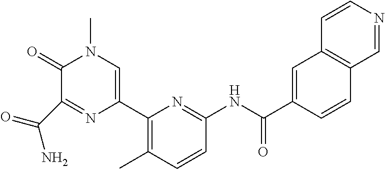 [SMILES: Cc1ccc(NC(=O)c2ccc3cnccc3c2)nc1-c1cn(C)c(=O)c(C(N)=O)n1] |
| I-136 | 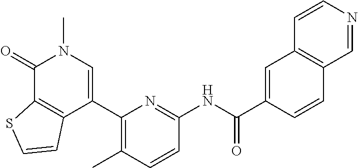 [SMILES: Cc1ccc(NC(=O)c2ccc3cnccc3c2)nc1-c1cn(C)c(=O)c2sccc12] |
| I-137 | 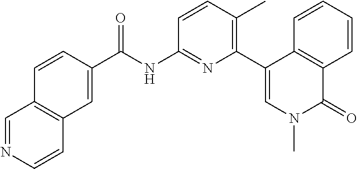 [SMILES: Cc1ccc(NC(=O)c2ccc3cnccc3c2)nc1-c1cn(C)c(=O)c2ccccc12] |
| I-138 | 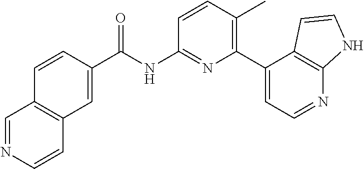 [SMILES: Cc1ccc(NC(=O)c2ccc3cnccc3c2)nc1-c1ccnc2[nH]ccc12] |
| I-139 | 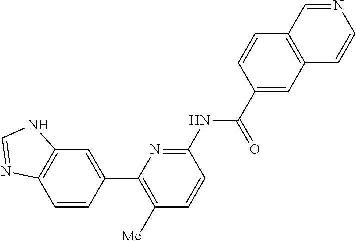 [SMILES: Cc1ccc(NC(=O)c2ccc3cnccc3c2)nc1-c1ccc2nc[nH]c2c1] |
| I-140 | 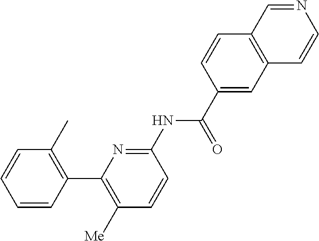 [SMILES: Cc1ccccc1-c1nc(NC(=O)c2ccc3cnccc3c2)ccc1C] |
| I-141 | 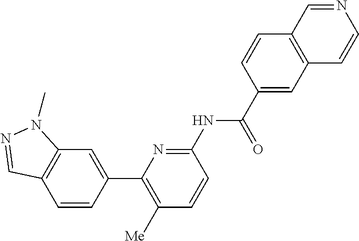 [SMILES: Cc1ccc(NC(=O)c2ccc3cnccc3c2)nc1-c1ccc2cnn(C)c2c1] |
| I-142 | 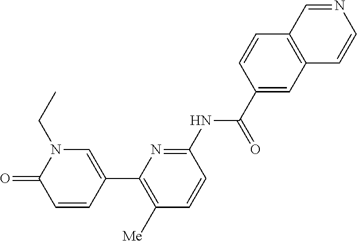 [SMILES: CCn1cc(-c2nc(NC(=O)c3ccc4cnccc4c3)ccc2C)ccc1=O] |
| I-143 | 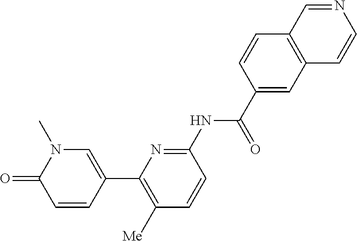 [SMILES: Cc1ccc(NC(=O)c2ccc3cnccc3c2)nc1-c1ccc(=O)n(C)c1] |
| I-144 | 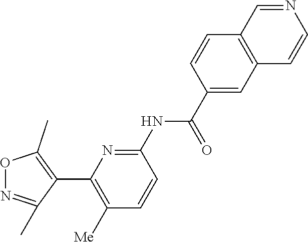 [SMILES: Cc1ccc(NC(=O)c2ccc3cnccc3c2)nc1-c1c(C)noc1C] |
| I-145 | 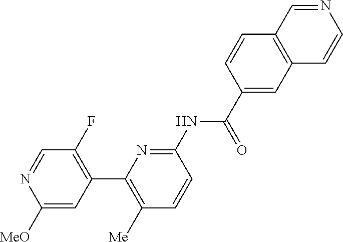 [SMILES: COc1cc(-c2nc(NC(=O)c3ccc4cnccc4c3)ccc2C)c(F)cn1] |
| I-146 | 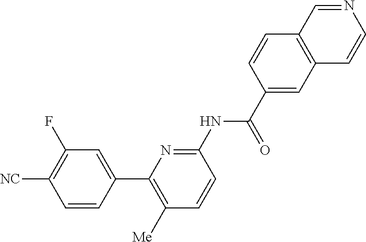 [SMILES: Cc1ccc(NC(=O)c2ccc3cnccc3c2)nc1-c1ccc(C#N)c(F)c1] |
| I-147 | 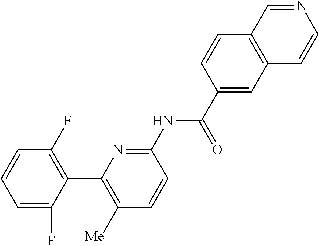 [SMILES: Cc1ccc(NC(=O)c2ccc3cnccc3c2)nc1-c1c(F)cccc1F] |
| I-148 | 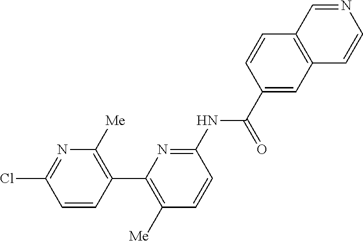 [SMILES: Cc1ccc(NC(=O)c2ccc3cnccc3c2)nc1-c1ccc(Cl)nc1C] |
| I-149 | 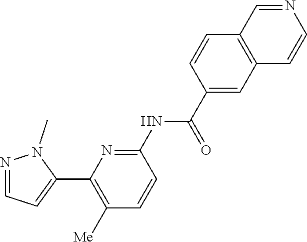 [SMILES: Cc1ccc(NC(=O)c2ccc3cnccc3c2)nc1-c1ccnn1C] |
| I-150 | 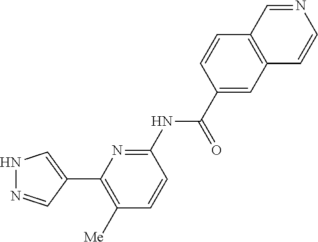 [SMILES: Cc1ccc(NC(=O)c2ccc3cnccc3c2)nc1-c1cn[nH]c1] |
| I-151 | 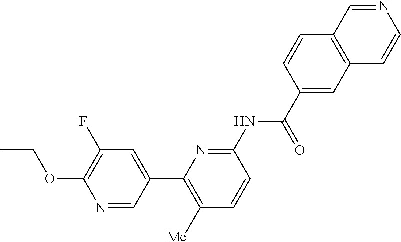 [SMILES: CCOc1ncc(-c2nc(NC(=O)c3ccc4cnccc4c3)ccc2C)cc1F] |
| I-152 | 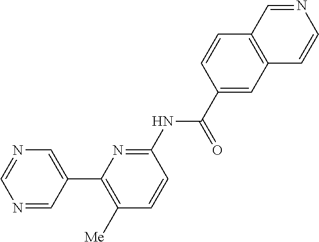 [SMILES: Cc1ccc(NC(=O)c2ccc3cnccc3c2)nc1-c1cncnc1] |
| I-153 | 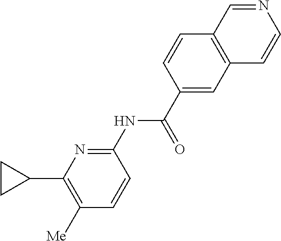 [SMILES: Cc1ccc(NC(=O)c2ccc3cnccc3c2)nc1C1CC1] |
| I-154 | 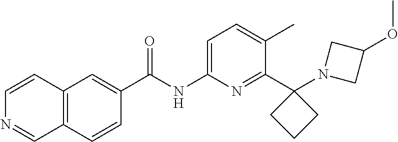 [SMILES: COC1CN(C2(c3nc(NC(=O)c4ccc5cnccc5c4)ccc3C)CCC2)C1] |
| I-155 | 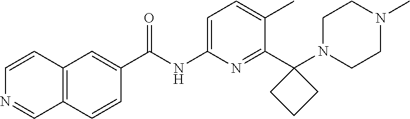 [SMILES: Cc1ccc(NC(=O)c2ccc3cnccc3c2)nc1C1(N2CCN(C)CC2)CCC1] |
| I-156 | 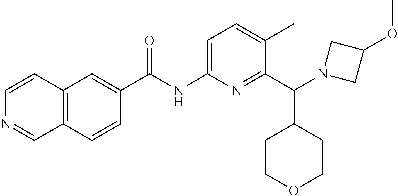 [SMILES: COC1CN(C(c2nc(NC(=O)c3ccc4cnccc4c3)ccc2C)C2CCOCC2)C1] |
| I-157 | 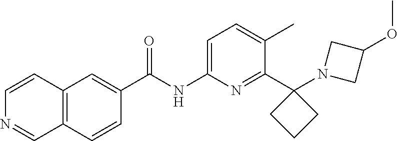 [SMILES: COC1CN(C2(c3nc(NC(=O)c4ccc5cnccc5c4)ccc3C)CCC2)C1] |
| I-158 | 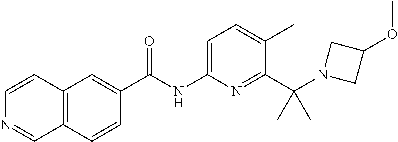 [SMILES: COC1CN(C(C)(C)c2nc(NC(=O)c3ccc4cnccc4c3)ccc2C)C1] |
| I-159 | 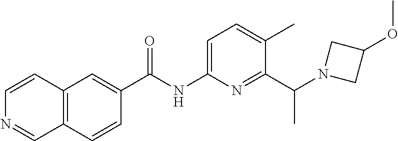 [SMILES: COC1CN(C(C)c2nc(NC(=O)c3ccc4cnccc4c3)ccc2C)C1] |
| I-160 | 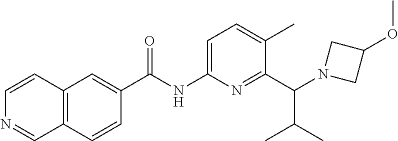 [SMILES: COC1CN(C(c2nc(NC(=O)c3ccc4cnccc4c3)ccc2C)C(C)C)C1] |
| I-161 | 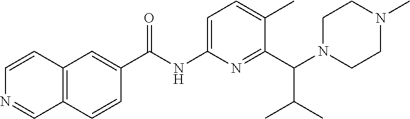 [SMILES: Cc1ccc(NC(=O)c2ccc3cnccc3c2)nc1C(C(C)C)N1CCN(C)CC1] |
| I-162 | 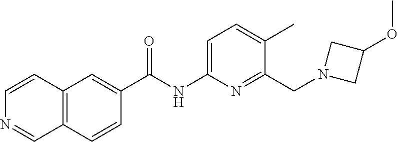 [SMILES: COC1CN(Cc2nc(NC(=O)c3ccc4cnccc4c3)ccc2C)C1] |
| I-163 | 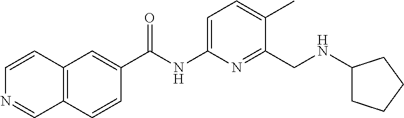 [SMILES: Cc1ccc(NC(=O)c2ccc3cnccc3c2)nc1CNC1CCCC1] |
| I-164 | 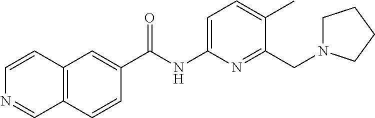 [SMILES: Cc1ccc(NC(=O)c2ccc3cnccc3c2)nc1CN1CCCC1] |
| I-165 | 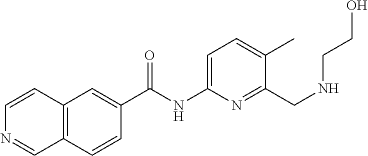 [SMILES: Cc1ccc(NC(=O)c2ccc3cnccc3c2)nc1CNCCO] |
| I-166 | 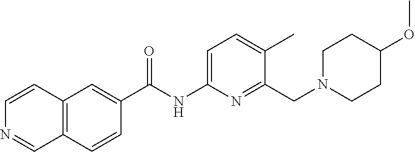 [SMILES: COC1CCN(Cc2nc(NC(=O)c3ccc4cnccc4c3)ccc2C)CC1] |
| I-167 | 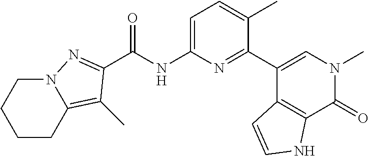 [SMILES: Cc1ccc(NC(=O)c2nn3c(c2C)CCCC3)nc1-c1cn(C)c(=O)c2[nH]ccc12] |
| I-168 | 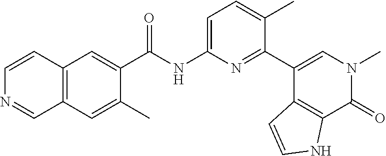 [SMILES: Cc1cc2cnccc2cc1C(=O)Nc1ccc(C)c(-c2cn(C)c(=O)c3[nH]ccc23)n1] |
| I-169 | 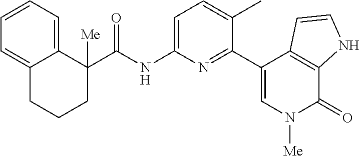 [SMILES: Cc1ccc(NC(=O)C2(C)CCCc3ccccc32)nc1-c1cn(C)c(=O)c2[nH]ccc12] |
| I-170 | 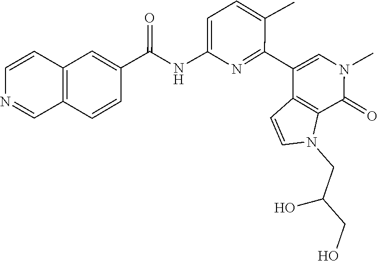 [SMILES: Cc1ccc(NC(=O)c2ccc3cnccc3c2)nc1-c1cn(C)c(=O)c2c1ccn2CC(O)CO] |
| I-171 | 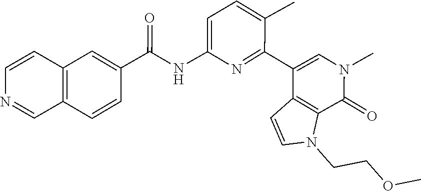 [SMILES: COCCn1ccc2c(-c3nc(NC(=O)c4ccc5cnccc5c4)ccc3C)cn(C)c(=O)c21] |
| I-172 | 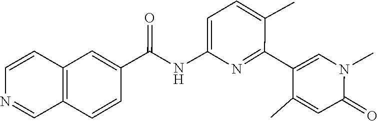 [SMILES: Cc1cc(=O)n(C)cc1-c1nc(NC(=O)c2ccc3cnccc3c2)ccc1C] |
| I-173 | 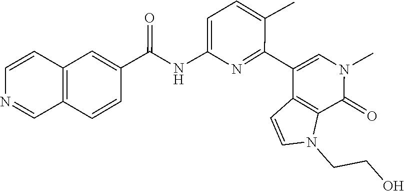 [SMILES: Cc1ccc(NC(=O)c2ccc3cnccc3c2)nc1-c1cn(C)c(=O)c2c1ccn2CCO] |
| I-174 | 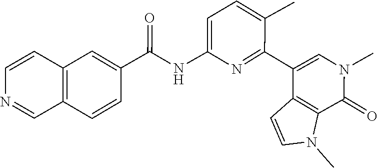 [SMILES: Cc1ccc(NC(=O)c2ccc3cnccc3c2)nc1-c1cn(C)c(=O)c2c1ccn2C] |
| I-175 | 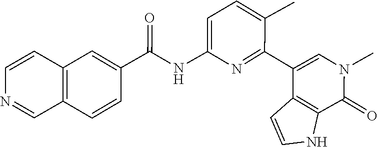 [SMILES: Cc1ccc(NC(=O)c2ccc3cnccc3c2)nc1-c1cn(C)c(=O)c2[nH]ccc12] |
| I-176 | 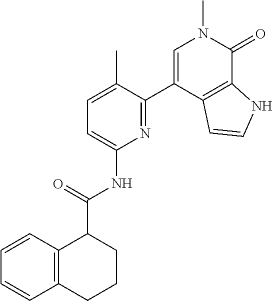 [SMILES: Cc1ccc(NC(=O)C2CCCc3ccccc32)nc1-c1cn(C)c(=O)c2[nH]ccc12] |
| I-177 | 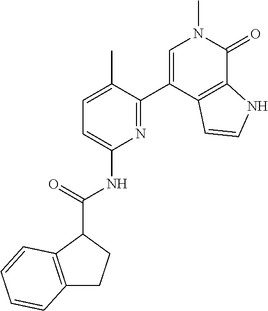 [SMILES: Cc1ccc(NC(=O)C2CCc3ccccc32)nc1-c1cn(C)c(=O)c2[nH]ccc12] |
| I-178 | 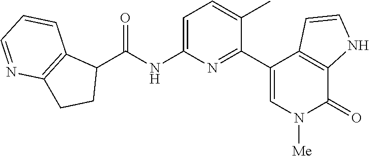 [SMILES: Cc1ccc(NC(=O)C2CCc3ncccc32)nc1-c1cn(C)c(=O)c2[nH]ccc12] |
| I-179 | 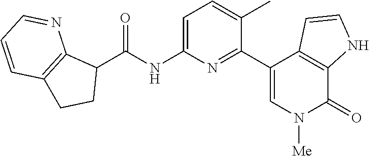 [SMILES: Cc1ccc(NC(=O)C2CCc3cccnc32)nc1-c1cn(C)c(=O)c2[nH]ccc12] |
| I-180 | 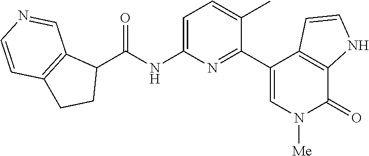 [SMILES: Cc1ccc(NC(=O)C2CCc3ccncc32)nc1-c1cn(C)c(=O)c2[nH]ccc12] |
| I-181 | 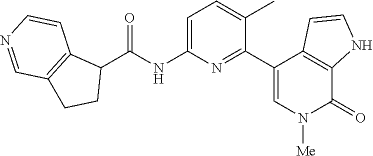 [SMILES: Cc1ccc(NC(=O)C2CCc3cnccc32)nc1-c1cn(C)c(=O)c2[nH]ccc12] |
| I-182 | 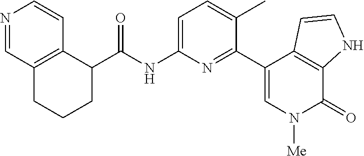 [SMILES: Cc1ccc(NC(=O)C2CCCc3cnccc32)nc1-c1cn(C)c(=O)c2[nH]ccc12] |
| I-183 | 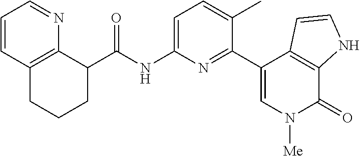 [SMILES: Cc1ccc(NC(=O)C2CCCc3cccnc32)nc1-c1cn(C)c(=O)c2[nH]ccc12] |
| I-184 | 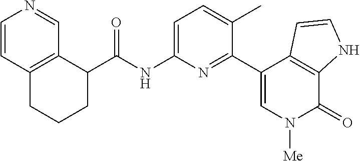 [SMILES: Cc1ccc(NC(=O)C2CCCc3ccncc32)nc1-c1cn(C)c(=O)c2[nH]ccc12] |
| I-185 | 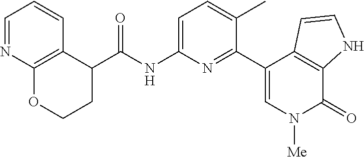 [SMILES: Cc1ccc(NC(=O)C2CCOc3ncccc32)nc1-c1cn(C)c(=O)c2[nH]ccc12] |
| I-186 | 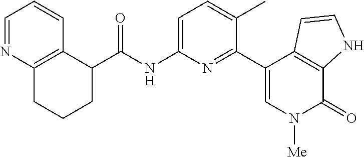 [SMILES: Cc1ccc(NC(=O)C2CCCc3ncccc32)nc1-c1cn(C)c(=O)c2[nH]ccc12] |
| I-187 | 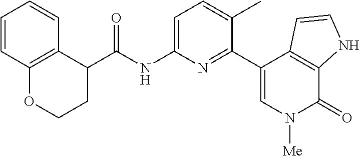 [SMILES: Cc1ccc(NC(=O)C2CCOc3ccccc32)nc1-c1cn(C)c(=O)c2[nH]ccc12] |
| I-188 | 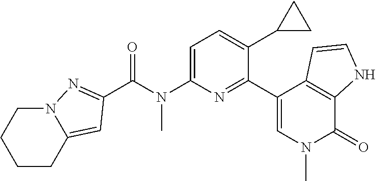 [SMILES: CN(C(=O)c1cc2n(n1)CCCC2)c1ccc(C2CC2)c(-c2cn(C)c(=O)c3[nH]ccc23)n1] |
| I-189 | 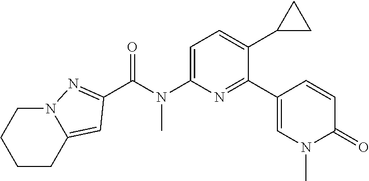 [SMILES: CN(C(=O)c1cc2n(n1)CCCC2)c1ccc(C2CC2)c(-c2ccc(=O)n(C)c2)n1] |
| I-190 | 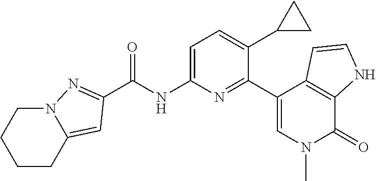 [SMILES: Cn1cc(-c2nc(NC(=O)c3cc4n(n3)CCCC4)ccc2C2CC2)c2cc[nH]c2c1=O] |
| I-191 | 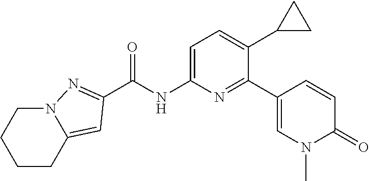 [SMILES: Cn1cc(-c2nc(NC(=O)c3cc4n(n3)CCCC4)ccc2C2CC2)ccc1=O] |
| I-192 | 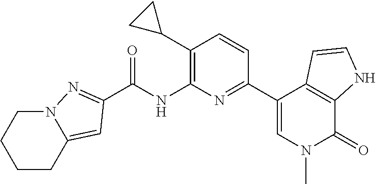 [SMILES: Cn1cc(-c2ccc(C3CC3)c(NC(=O)c3cc4n(n3)CCCC4)n2)c2cc[nH]c2c1=O] |
| I-193 | 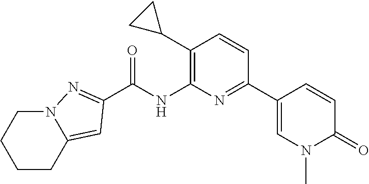 [SMILES: Cn1cc(-c2ccc(C3CC3)c(NC(=O)c3cc4n(n3)CCCC4)n2)ccc1=O] |
| I-194 | 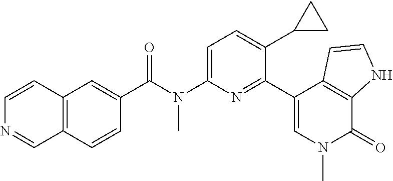 [SMILES: CN(C(=O)c1ccc2cnccc2c1)c1ccc(C2CC2)c(-c2cn(C)c(=O)c3[nH]ccc23)n1] |
| I-195 | 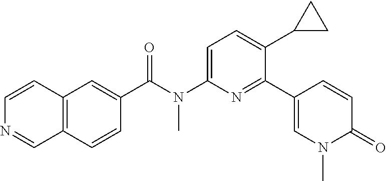 [SMILES: CN(C(=O)c1ccc2cnccc2c1)c1ccc(C2CC2)c(-c2ccc(=O)n(C)c2)n1] |
| I-196 | 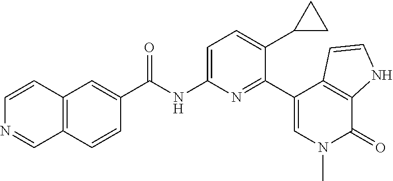 [SMILES: Cn1cc(-c2nc(NC(=O)c3ccc4cnccc4c3)ccc2C2CC2)c2cc[nH]c2c1=O] |
| I-197 | 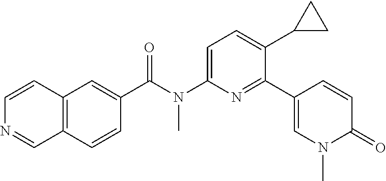 [SMILES: CN(C(=O)c1ccc2cnccc2c1)c1ccc(C2CC2)c(-c2ccc(=O)n(C)c2)n1] |
| I-198 | 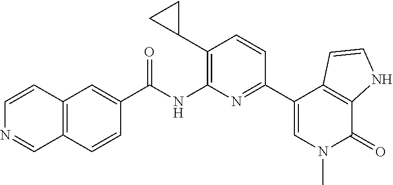 [SMILES: Cn1cc(-c2ccc(C3CC3)c(NC(=O)c3ccc4cnccc4c3)n2)c2cc[nH]c2c1=O] |
| I-199 | 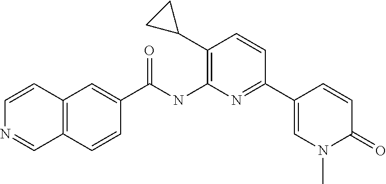 [SMILES: Cn1cc(-c2ccc(C3CC3)c(NC(=O)c3ccc4cnccc4c3)n2)ccc1=O] |

In some embodiments, the present invention provides a compound of formula I as defined above, or a pharmaceutically acceptable salt thereof, or a pharmaceutical composition comprising a compound of formula I as defined above, or a pharmaceutically acceptable salt thereof, and a pharmaceutically acceptable carrier, adjuvant, or vehicle for use as a medicament.

In some embodiments, the present invention provides a compound set forth in Table 1, above, or a pharmaceutically acceptable salt thereof. In some embodiments, the present invention provides a compound set forth in Table 1, above. In some embodiments, the present invention provides a pharmaceutical composition comprising a compound set forth in Table 1 above, or a pharmaceutically acceptable salt thereof, together with a pharmaceutically acceptable carrier, excipient, or diluent.

### II. Therapeutic Applications

In some embodiments, the invention provides a method of treating a disorder in which enhanced ABC transporter function is of clinical benefit. For instance, in certain embodiments, a disorder is one in which ABC transporter dysfunction is etiological for disease. In some embodiments, correction of one or more underlying mutations associated with ABC transporter dysfunction is rationalized. In some embodiments, methods of the present invention provide enhancement of one or more non-mutated forms of an ABC transporter.

In some embodiments, the invention provides a method of treating a disorder in which enhanced ABC transporter function is of clinical benefit, wherein the method comprises administering to a subject in need thereof a therapeutically effective amount of a compound described herein, such as a compound of Formula I, to treat the disorder in which enhanced ABC transporter function is of clinical benefit. In certain embodiments, the particular compound of Formula I is a compound defined by one of the embodiments described in Section I, above.

In some embodiments, the invention provides a method of treating a disorder associated with ABC transporter dysfunction, wherein the method comprises administering to a subject in need thereof a therapeutically effective amount of a compound described herein, such as a compound of Formula I, to treat the disorder associated with ABC transporter dysfunction. In certain embodiments, the particular compound of Formula I is a compound defined by one of the embodiments described in Section I, above.

In some embodiments, the present invention provides a method of alleviating one or more symptoms of a disorder associated with ABC transporter dysfunction, wherein the method comprises administering to a subject in need thereof a therapeutically effective amount of a compound described herein, such as a compound of Formula I, to treat the disorder associated with ABC transporter dysfunction. In certain embodiments, the particular compound of Formula I is a compound defined by one of the embodiments described in Section I, above.

In some embodiments, the disorder associated with ABC transporter dysfunction is characterized by dysfunction in a transporter selected from one or more of ABCA1, ABCA2, ABCA3, ABCA4, ABCA5, ABCA7, ABCA12, ABCB2, ABCB3, ABCB4, ABCB6, ABCB7, ABCB10, ABCB11, ABCC1, ABCC2, ABCC4, ABCC5, ABCC6 ABCC7, ABCC8, ABCC9, ABCC12, ABCD1, ABCD2, ABCD3, ABCD, ABCG5, ABCG8, ABCG1, and ABCG4.

In some embodiments, the invention provides methods of treating a disorder selected from Tangier disease, Surfactant metabolism dysfunction pulmonary 3, autosomal recessive Ichthyosis congenital 4A (ARCI). Bare lymphocyte syndrome type I, Bare lymphocyte syndrome type I due to TAP2 deficiency, Dyschromatosis universalis hereditaria 3, X-linked sideroblastic anemia with ataxia, Dubin-Johnson Syndrome, Cystic fibrosis (CF), Familial Hyperinsulinemic Hypoglycemia 1, Intellectual disability Myopathy Syndrome, Congenital bile acid synthesis defect 5, Methylmalonic aciduria and homocystinuria cblJ type. Sitostrolemia, Stargardt disease, PFIC3, PFIC2, Pseudoxanthoma Elasticum, X-linked adrenoleukodystrophy (ALD), Cholestasis, Hyperbilirubinemia, Intrahepatic cholestasis of pregnancy, Biliary atresia, Alagille syndrome, primary biliary cholangitis, primary sclerosing cholangitis. NAFLD/NASH, Alzheimer's disease. Huntington's disease, Multiple sclerosis, Parkinson's disease. Hirschsprung disease, Zellweger syndrome, Type 2 diabetes, Obesity, Type 1 diabetes, Atherosclerosis, Dyslipidemia, Generalized arterial calcification of infancy, Calciphylaxis, Autosomal recessive cone-rod dystrophy, Gout, PFIC1, Myo5B deficiency cholestasis, PFIC4, and Low phospholipid associated cholelithiasis.

In some embodiments, the disorder associated with ABC transporter dysfunction is cystic fibrosis (CF). Accordingly, in some embodiments, the present invention provides a method of treating cystic fibrosis, wherein the method comprises administering to a subject in need thereof a therapeutically effective amount of a compound described herein, such as a compound of Formula I. In some such embodiments, the method further comprises administering one or more additional therapeutic agents, described further below and herein.

In some embodiments, the disorder associated with ABC transporter dysfunction is cholestasis. Accordingly, in some embodiments, the present invention provides a method of treating cholestasis, wherein the method comprises administering to a subject in need thereof a therapeutically effective amount of a compound described herein, such as a compound of Formula I. One of skill in the medical arts will recognize that there are various forms of cholestasis, all of which are contemplated herein for treatment with methods and compounds of the present invention. In some embodiments, the cholestasis is intrahepatic. In some embodiments, the cholestasis is extrahepatic. In some embodiments, the cholestasis is any of those described above and herein.

### Subjects

In certain embodiments, the subject is a human. In certain embodiments, the subject is an adult human. In certain embodiments, the subject is a pediatric human.

### Medical Uses

Another aspect of the invention provides for the use of a compound described herein (such as a compound of Formula I, or other compounds in Section I) in the manufacture of a medicament. In certain embodiments, the medicament is for treating a disorder described herein, such as a disorder associated with ABC transporter dysfunction. Exemplary such disorders are described above and herein.

Another aspect of the invention provides for the use of a compound described herein (such as a compound of Formula I, or other compounds in Section I) for treating a medical disorder, such as a disorder associated with ABC transporter dysfunction. Exemplary such disorders are described above and herein.

### III. Combination Therapy

Another aspect of the invention provides for combination therapy. Compounds described herein (such as a compound of Formula I, or other compounds in Section I) or their pharmaceutically acceptable salts may be used in combination with additional therapeutic agents to treat medical disorders, such as an autoimmune disorder, cancer, etc.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 19, 20, 21, 22, 23, or 24 hours from one another. In some embodiments, one or more other therapeutic agent and a compound or composition of the invention are administered as a multiple dosage regimen more than 24 hours apart.

In some embodiments, the present invention provides a method of treating cystic fibrosis (CF) comprising administering a compound of the present invention with one or more additional therapeutic agents. In some embodiments, the one or more additional therapeutic agents are selected from a mucolytic agent, a bronchodialator, an antibiotic, an anti-infective agent, an anti-inflammatory agent, a cystic fibrosis transmembrane conductance (CFTR) modulator, a nutritional agent, or any agent known to treat CF.

In some embodiments, the one or more additional therapeutic agent is an S-nitrosoglutathione reductase (GSNOR) inhibitor. In some embodiments, the GSNOR inhibitor is selected from a GSNOR inhibitor disclosed in WO2010/019903, U.S. Pat. Nos. 8,470,857, 8,642,628, WO2010/019910, U.S. Pat. No. 8,586,624, WO2011/100433, U.S. Pat. No. 8,481,590, WO2012/048181. WO2012/083165, WO2012/083171, or WO 2012/170371.

In some embodiments, the one or more additional therapeutic agents is an ileal bile transport (IBAT) inhibitor. In some embodiments, the IBAT inhibitor is selected from an IBAT inhibitor disclosed in AU2011326873, US2020/0330545, WO2012/064266, WO2020/167964, or Front. Pharmacol. 2018: 9: 931 (Al-Dury et al., published online Aug. 21, 2018). Exemplary IBAT inhibitors include, but are not limited to odevixibat, elobixivat, maralixibat, linerixibat, GSK2330672, SHP626 (volixibat), A4250, etc.

### Additional Considerations

The doses and dosage regimen of the active ingredients used in the combination therapy may be determined by an attending clinician. In certain embodiments, the compound described herein (such as a compound of Formula I, or other compounds in Section I) and the additional therapeutic agent(s) are administered in doses commonly employed when such agents are used as monotherapy for treating the disorder. In other embodiments, the compound described herein (such as a compound of Formula I, or other compounds in Section I) and the additional therapeutic agent(s) are administered in doses lower than the doses commonly employed when such agents are used as monotherapy for treating the disorder. In certain embodiments, the compound described herein (such as a compound of Formula I, or other compounds in Section I) and the additional therapeutic agent(s) are present in the same composition, which is suitable for oral administration.

In certain embodiments, the compound described herein (such as a compound of Formula I, or other compounds in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of the compound described herein (such as a compound of Formula I, or other compounds in Section I), a pharmaceutically acceptable carrier, vehicle or diluent, and optionally at least one additional therapeutic agent listed above.

### III. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (such as a compound of Formula I, or other compounds in Section I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like: (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxmethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a compound described herein in a therapeutically effective amount for the treatment of a medical disorder described herein.

### EXAMPLES

The invention now being generally described, will be more readily understood by reference to the following examples, which are included merely for purposes of illustration of certain aspects and embodiments of the present invention, and is not intended to limit the invention.

### Synthetic Methods

Methods for preparing compounds described herein are illustrated in the following synthetic Scheme. The Scheme is given for the purpose of illustrating the invention, and not intended to limit the scope or spirit of the invention. Starting materials shown in the Scheme can be obtained from commercial sources or can be prepared based on procedures described in the literature.

In the Schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in “Protecting Groups in Organic Synthesis”, T. W. Greene and P. G. M. Wuts, 3^{rd} edition, John Wiley & Sons, 1999, the entire contents of which are hereby incorporated by reference. The modular synthetic route illustrated in Scheme 1 can also be readily modified by one of skill in the art to provide additional compounds by conducting functional group transformations on the intermediate and final compounds. Such functional group transformations are well known in the art, as described in, for example. “Comprehensive Organic Synthesis” (B. M. Trost & I. Fleming, eds., 1991-1992).

### HPLC Method 1:

Instrument: Shimadzu LCMS system equipped with UV and MS detector
Mobile Phase: 0.225% formic acid in water (solvent A) and CH₃CN (solvent B),
Gradient: using the elution gradient 30%-70% (solvent B) over 10 minutes.
Flow rate: 35 ml/min;
Column: Phenomenex Gemin-NX 150*30 mm*5 um
Wavelength: UV 220 nm and 254 nm)
Column temperature: 30 C;
MS ionization: ESI

### HPLC Method 2:

Instrument: GILSON 215 and Shimadzu LCMS 2010A
Column: Agela Durashell C18 250*20 mm*5 um
Mobile phase A: 0.1% ammonium bicarbonate in water (m/m)
Mobile phase B: Acetonitrile
Gradient: 80% A/20% B linear to 60% A/40% B in 10.0 min, hold at 0% A/100% B to 12.0 min.
Flow rate: 35 mL/min or 30 mL/min

### HPLC Method 3:

Instrument: Shimadzu LCMS system equipped with DAD detector
Mobile Phase: 1.5 mL/4 L TFA in water (solvent A) and 0.75 mL/4 L TFA in CH₃CN (solvent B),
Gradient: using the elution gradient 5%-95% (solvent B) over 0.7 minutes and holding at 95% for 0.4 minutes at a flow rate of 1.5 ml/min;
Column: Chromolish.Flash RP-18e 25-2 mm
Wavelength: UV 220 nm (or 215 nm, 254 nm)
Column temperature: 50° C.;
MS ionization: ESI

### LC-MS Method A:

ESI+/− ion mode 150-850 Da
Wavelength: UV 220 nm
Column: Waters, Xbridge C18 50*2.1 mm, Sum
Temperature: 40° C.

### Gradient:

| Time | 0.038% TFA in | 0.019% TFA in | Flow |
|---|---|---|---|
| (min) | water | acetonitrile | (mL/min) |
| 0 | 99% | 1% | 0.8 |
| 3.85 | 0% | 100% | 0.8 |

### LC-MS Method B:

ESI+/− ion mode 150-850 Da
Wavelength: UV 220 nm
Column: Waters, Xbridge C18 50*2.1 mm, 5 um
Temperature: 40° C.

### Gradient:

| Time | 0.038% TFA in | 0.019% TFA in | Flow |
|---|---|---|---|
| (min) | water | acetonitrile | (mL/min) |
| 0 | 90% | 10% | 0.8 |
| 3.85 | 0% | 100% | 0.8 |

### LC-MS Method C:

ESI+/− ion mode 150-850 Da
Wavelength: UV 220 nm
Column: Waters, Xbridge C18 50*2.1 mm, 5 um
Temperature: 40° C.

### Gradient:

| Time | 0.05% ammonium hydroxide | | Flow |
|---|---|---|---|
| (min) | in water | acetonitrile | (mL/min) |
| 0 | 95% | 5% | 0.8 |
| 3.60 | 0% | 100% | 0.8 |

### LC-MS Method D:

ESI+/− ion mode 150-850 Da
Wavelength: UV 220 nm
Column: Welch Boltimate® Core-Shell
Temperature: 50° C.

### Gradient:

| Time | 0.037% TFA in | 0.018% TFA in | Flow |
|---|---|---|---|
| (min) | water | acetonitrile | (mL/min) |
| 0 | 95% | 5% | 1.5 |
| 0.4 | 5% | 95% | 1.5 |
| 0.7 | 5% | 95% | 1.5 |

### Example 1—Synthesis of Intermediate A-1

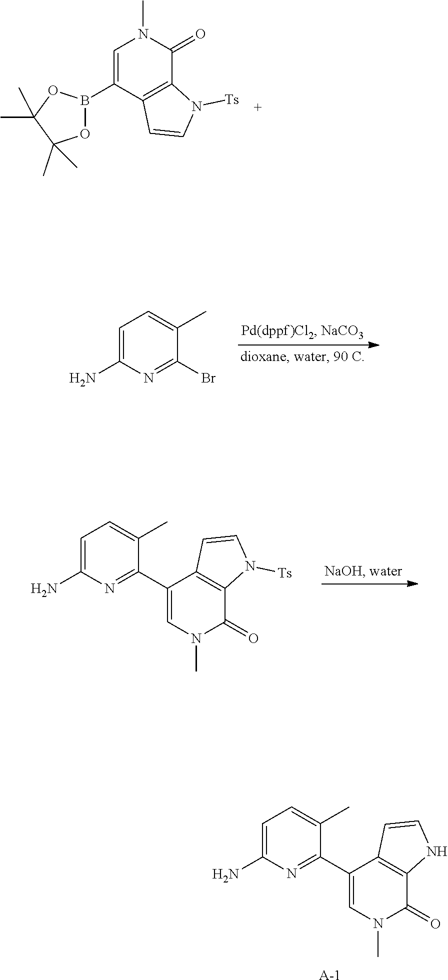

A solution of 6-methyl-4-(4,4,5,5-tetramethyl-1,3,2-dioxaborolan-2-yl)-1-tosyl-1,6-dihydro-7H-pyrrolo[2,3-c]pyridin-7-one (prepared via WO2018/188047, 2018, A1, 8.00 g, 18.7 mmol), 6-bromo-5-methylpyridin-2-amine (3.84 g, 20.6 mmol), and Na₂CO₃ (5.94 g, 56.1 mmol, 3.00 eq) in H₂O (20 mL) and dioxane (70 mL) was degassed and purged with N₂, and then Pd(dppf)Cl₂ (609 mg. 0.93 mmol) was added. The mixture was stirred at 90° C. for 0.5 h under N₂ atmosphere then was concentrated under reduced pressure to give a residue that was purified by flash chromatography (SiO₂, 0˜40% ethyl acetate in petroleum ether) to afford 4-(6-amino-3-methylpyridin-2-yl)-6-methyl-1-tosyl-1,6-dihydro-7H-pyrrolo[2,3-c]pyridin-7-one (7.50 g, 98%). (m/z=409.1, ret time=0.43 min, Analytical LC/MS Analysis Method D)

To a solution of 4-(6-amino-3-methylpyridin-2-yl)-6-methyl-1-tosyl-1,6-dihydro-7H-pyrrolo[2,3-c]pyridin-7-one (15.0 g, 36.7 mmol) in EtOH (160 mL) was added aqueous NaOH (3 M, 142 mL) solution. The mixture was stirred at 25° C. for 16 h then concentrated under reduced pressure to give a residue that was extracted with ethyl acetate (50 mL×3), dried over anhydrous MgSO₄, filtered and concentrated under reduced pressure. The residue was purified by flash chromatography (SiO₂, 0˜20% ethyl acetate in petroleum ether) to afford A-1 (4.0 g, 43%). (m/z=255, ret time=0.40 min, Analytical LC/MS Analysis Method D)

### Example 2—Method A: Synthesis of Compound I-26

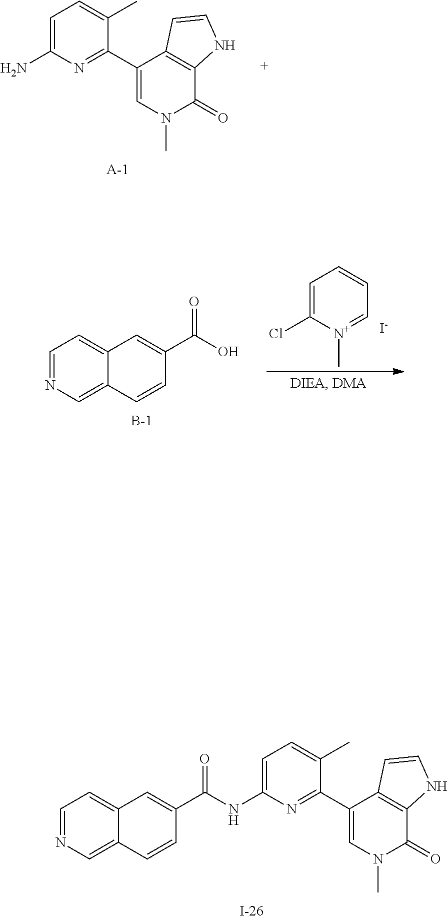

To a solution of A-1 (25 mg, 0.1 mmol) in DMA (1 mL) was added B-1 (17 mg, 0.1 mmol) and DIEA (39 mg, 0.3 mmol). To this mixture was added 2-chloro-1-methylpyridinium iodide (38 mg, 0.15 mmol), and the reaction mixture was then heated at 50° C. for 16 h, then concentrated under reduced pressure, and purified by prep HPLC (Method 1) to give I-26 (20 mg, 49%).

### Example 3—Synthesis of Additional Compounds by Method A

The following compounds were made using synthetic methods analogous to those used in Method A, using A-1 and the appropriate carboxylic acids: Compounds I-24, I-23, I-22, I-21, I-20, I-19, I-18, I-6, and I-3.

### Example 4—Method B: Synthesis of Compound I-27

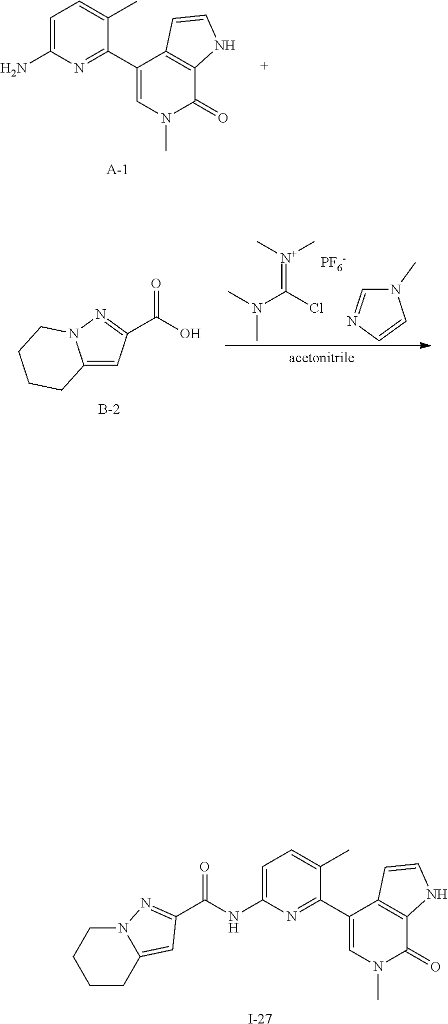

To a solution of A-1 (25 mg, 0.1 mmol) in acetonitrile was added B-2 (17 mg. 0.1 mmol) and N-methylimidazole (19 mg, 0.23 mmol), followed by chloro-N,N,N′,N′-tetramethylformamidinium hexafluorophosphate (42 mg, 0.15 mmol). The mixture was stirred at 50° C. for 16 h then concentrated under reduced pressure and purified by prep HPLC (Method 1) to give Compound I-27 (20 mg, 50%).

### Example 5—Method C: Synthesis of Compound I-15

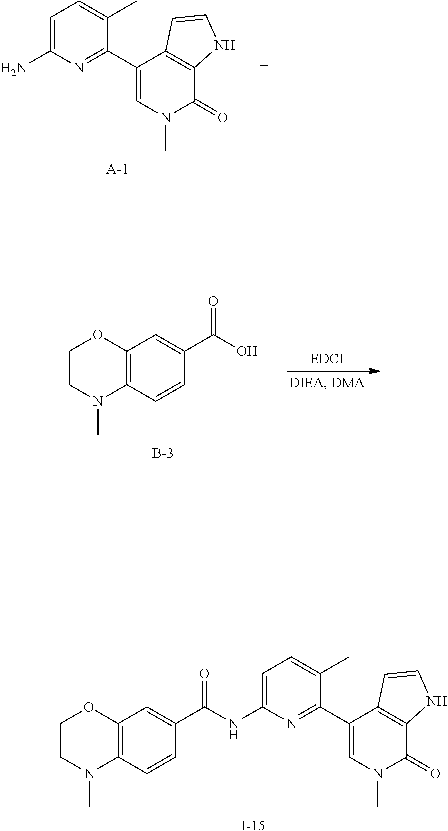

To a solution of A-1 (25 mg, 0.1 mmol) in pyridine (1 mL) was added B-3 (20 mg, 0.1 mmol) followed by EDCI (19 mg, 0.12 mmol). The mixture was stirred at 65° C. for 2 h then concentrated and purified by prep HPLC (Method 1) to give Compound I-15 (6.3 mg, 15%).

### Example 6—Synthesis of Additional Compounds by Method C

The following compounds were made using synthetic methods analogous to those used in Method C, using A-1 and the appropriate carboxylic acids: Compounds I-16, I-14, I-10, I-9, I-8, I-7, I-5, I-4, I-177, I-1 I-180, I-2, I-11, I-12, I-13, I-25, I-17, I-176.

### Example 7—Compound Characterization

Physical characterization data for exemplary compounds is provided below.

TABLE 2
Compound Characterization Data

| Compound | | | Ret Time | LC-MS |
|---|---|---|---|---|
| No. | Structure | Mol ion | (min) | Method |
| I-27 | 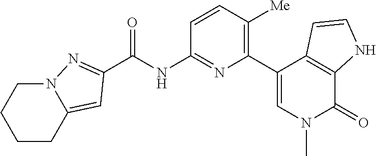 [SMILES: Cc1ccc(NC(=O)c2cc3n(n2)CCCC3)nc1-c1cn(C)c(=O)c2[nH]ccc12] | 403.3 | 2.479 | A |
| I-26 | 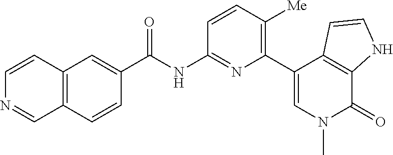 [SMILES: Cc1ccc(NC(=O)c2ccc3cnccc3c2)nc1-c1cn(C)c(=O)c2[nH]ccc12] | 410.3 | 2.19 | A |
| I-24 | 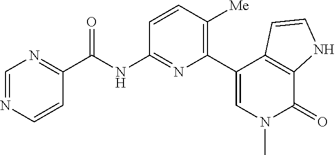 [SMILES: Cc1ccc(NC(=O)c2ccncn2)nc1-c1cn(C)c(=O)c2[nH]ccc12] | 361.2 | 1.971 | C |
| I-23 | 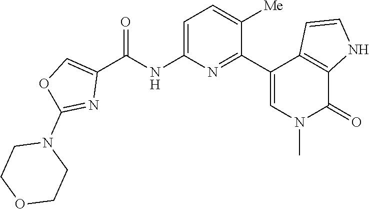 [SMILES: Cc1ccc(NC(=O)c2coc(N3CCOCC3)n2)nc1-c1cn(C)c(=O)c2[nH]ccc12] | 425.2 | 2.507 | A |
| I-22 | 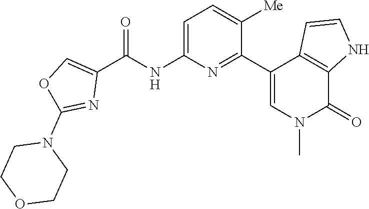 [SMILES: Cc1ccc(NC(=O)c2coc(N3CCOCC3)n2)nc1-c1cn(C)c(=O)c2[nH]ccc12] | 435.2 | 2.176 | C |
| I-21 | 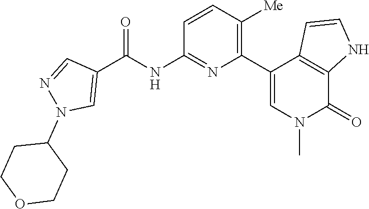 [SMILES: Cc1ccc(NC(=O)c2cnn(C3CCOCC3)c2)nc1-c1cn(C)c(=O)c2[nH]ccc12] | 433.2 | 1.995 | C |
| I-20 | 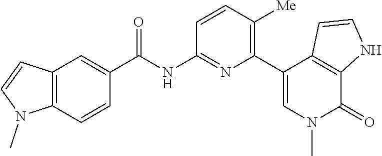 [SMILES: Cc1ccc(NC(=O)c2ccc3c(ccn3C)c2)nc1-c1cn(C)c(=O)c2[nH]ccc12] | 412.2 | 2.509 | A |
| I-19 | 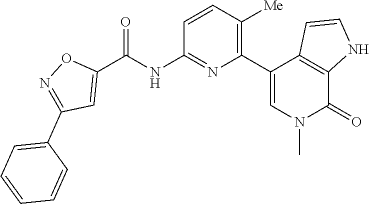 [SMILES: Cc1ccc(NC(=O)c2cc(-c3ccccc3)no2)nc1-c1cn(C)c(=O)c2[nH]ccc12] | 426.1 | 3.176 | A |
| I-18 | 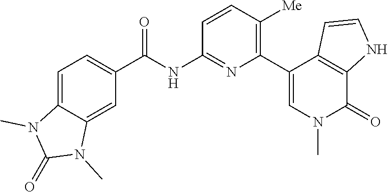 [SMILES: Cc1ccc(NC(=O)c2ccc3c(c2)n(C)c(=O)n3C)nc1-c1cn(C)c(=O)c2[nH]ccc12] | 443.1 | 2.598 | A |
| I-16 | 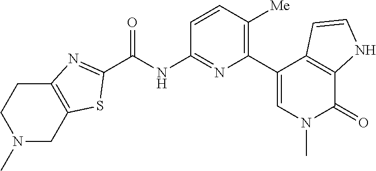 [SMILES: Cc1ccc(NC(=O)c2nc3c(s2)CN(C)CC3)nc1-c1cn(C)c(=O)c2[nH]ccc12] | 435.2 | 2.204 | C |
| I-15 | 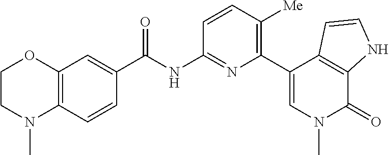 [SMILES: Cc1ccc(NC(=O)c2ccc3c(c2)OCCN3C)nc1-c1cn(C)c(=O)c2[nH]ccc12] | 430.2 | 2.408 | A |
| I-14 | 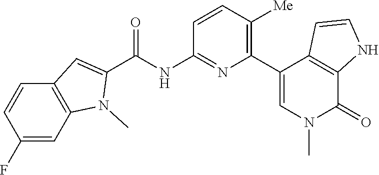 [SMILES: Cc1ccc(NC(=O)c2cc3ccc(F)cc3n2C)nc1-c1cn(C)c(=O)c2[nH]ccc12] | 430.2 | 2.544 | B |
| I-10 | 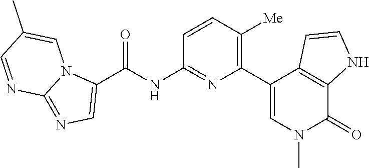 [SMILES: Cc1cnc2ncc(C(=O)Nc3ccc(C)c(-c4cn(C)c(=O)c5[nH]ccc45)n3)n2c1] | 414.2 | 2.302 | A |
| I-9 | 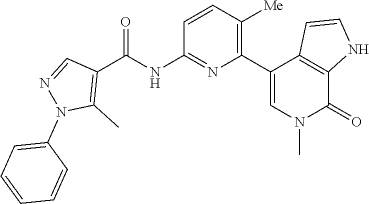 [SMILES: Cc1ccc(NC(=O)c2cnn(-c3ccccc3)c2C)nc1-c1cn(C)c(=O)c2[nH]ccc12] | 439.2 | 2.557 | A |
| I-8 | 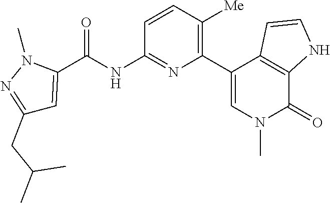 [SMILES: Cc1ccc(NC(=O)c2cc(CC(C)C)nn2C)nc1-c1cn(C)c(=O)c2[nH]ccc12] | 419.2 | 2.803 | A |
| I-7 | 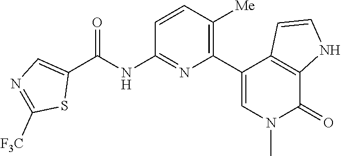 [SMILES: Cc1ccc(NC(=O)c2cnc(C(F)(F)F)s2)nc1-c1cn(C)c(=O)c2[nH]ccc12] | 434.1 | 2.855 | A |
| I-6 | 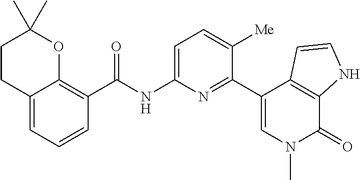 [SMILES: Cc1ccc(NC(=O)c2cccc3c2OC(C)(C)CC3)nc1-c1cn(C)c(=O)c2[nH]ccc12] | 443.1 | 3.014 | B |
| I-5 | 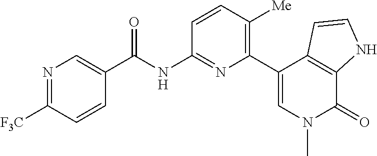 [SMILES: Cc1ccc(NC(=O)c2ccc(C(F)(F)F)nc2)nc1-c1cn(C)c(=O)c2[nH]ccc12] | 428.2 | 2.628 | A |
| I-4 | 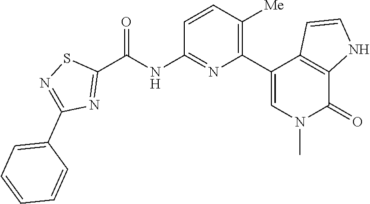 [SMILES: Cc1ccc(NC(=O)c2nc(-c3ccccc3)ns2)nc1-c1cn(C)c(=O)c2[nH]ccc12] | 443.2 | 3.239 | A |
| I-3 | 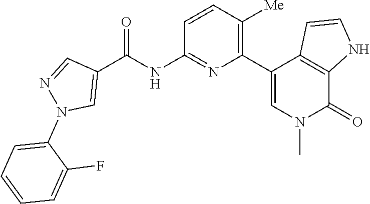 [SMILES: Cc1ccc(NC(=O)c2cnn(-c3ccccc3F)c2)nc1-c1cn(C)c(=O)c2[nH]ccc12] | 443.2 | 2.471 | A |
| I-1 | 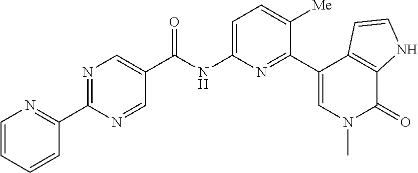 [SMILES: Cc1ccc(NC(=O)c2cnc(-c3ccccn3)nc2)nc1-c1cn(C)c(=O)c2[nH]ccc12] | 438.2 | 1.856 | C |
| I-17 | 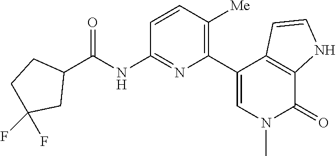 [SMILES: Cc1ccc(NC(=O)C2CCC(F)(F)C2)nc1-c1cn(C)c(=O)c2[nH]ccc12] | 387.2 | 2.047 | B |
| I-12 | 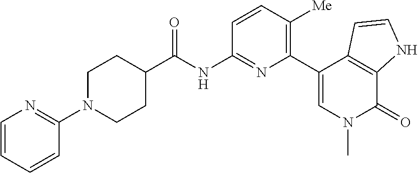 [SMILES: Cc1ccc(NC(=O)C2CCN(c3ccccn3)CC2)nc1-c1cn(C)c(=O)c2[nH]ccc12] | 443.3 | 2.085 | A |
| I-177 | 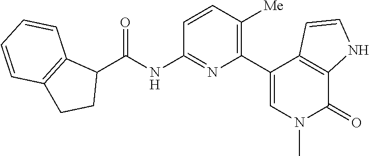 [SMILES: Cc1ccc(NC(=O)C2CCc3ccccc32)nc1-c1cn(C)c(=O)c2[nH]ccc12] | 399.2 | 2.634 | A |
| I-11 | 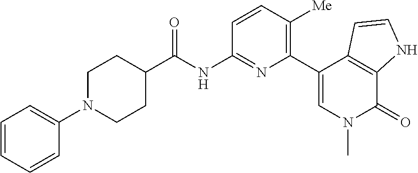 [SMILES: Cc1ccc(NC(=O)C2CCN(c3ccccc3)CC2)nc1-c1cn(C)c(=O)c2[nH]ccc12] | 442.3 | 2.171 | A |
| I-176 | 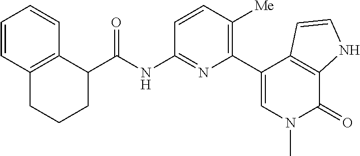 [SMILES: Cc1ccc(NC(=O)C2CCCc3ccccc32)nc1-c1cn(C)c(=O)c2[nH]ccc12] | 413.2 | 2.699 | A |
| I-2 | 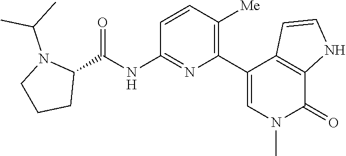 [SMILES: Cc1ccc(NC(=O)[C@@H]2CCCN2C(C)C)nc1-c1cn(C)c(=O)c2[nH]ccc12] | 394.1 | 2.093 | A |
| I-13 | 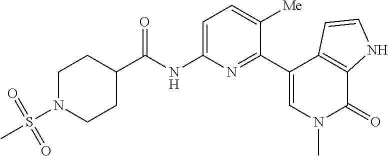 [SMILES: Cc1ccc(NC(=O)C2CCN(S(C)(=O)=O)CC2)nc1-c1cn(C)c(=O)c2[nH]ccc12] | 444.2 | 1.964 | C |
| I-25 | 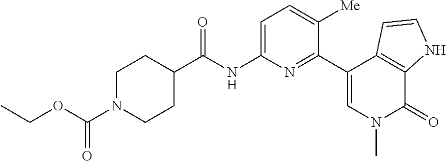 [SMILES: CCOC(=O)N1CCC(C(=O)Nc2ccc(C)c(-c3cn(C)c(=O)c4[nH]ccc34)n2)CC1] | 438.4 | 2.357 | A |

### Example 8—ABCB11 E297G Assay

To assess activity against ABCB11 E297G, the compounds were screened in an assay measuring protein expression, using cells transfected with cDNA encoding HiBit-tagged ABCB11 E297G.

Here we describe an assay for measuring the expression of the E297G variant of human ABCB11 (henceforth referred to as ABCB11 E297G) and the use of this assay to measure the efficacy of compounds to increase the expression of ABCB11 E297G. The assay was designed to characterize the efficacy and potency of compounds to increase the expression of ABCB11 E297G by calculating an Eₘₐₓ and EC₅₀ value. HEK-293 cells stably expressing E297G (HEK-293/E297G cells) were used for this assay and were maintained at 37° C. (5% CO₂, humidified) in Eagle's Minimum Essential Medium supplemented with 10% fetal bovine serum and 1% Glutamine. For the assay, the HEK-293/E297G cells were seeded in black/transparent-bottom 384-well plates at 20,000 cells/well in 22.5 μl Opti-MEM (Thermofisher, product #11058021) media supplemented with 2.7% of fetal bovine serum (Gibco, product #16000044) and allowed to settle for 4 hours at 37° C. (5% CO₂, humidified). Compound serial dilutions are prepared in Opti-MEM as four times (4×) the final concentration and using a dilution factor of 2.15. After the four h incubation period, 7.5 μL of the 4× dilutions are transferred to the assay plate.

Dose response curves include eight doses with the top final concentration at 25 μM in 0.75% DMSO. Liquid handling is performed in a robotic device equipped with a 384-tip head (Analytik Jena—CyBio Vario). After addition of compound treatment, cells are incubated for 24 hours at 37° C. (5% CO₂, humidified). After the 24-hour treatment, plates are allowed to equilibrate for 30 minutes at room temperature. Using the Analytik Jena—CyBio Vario instrument, the volume of each well is reduced to 15 μl and 15 μl of HiBit lytic assay reagent (Promega, product #N3040), which is prepared according to manufacturer's instructions, is added back to each well. The plates are incubated at room temperature for additional 30 minutes and luminescent signal is acquired using a Hamamatsu FDSS 7000 EX plate-reader (20 reads, exposure time 0.2 seconds, gain 200).

Results are presented below in Table 3 for ABCB11 E297G. Compounds having an activity designated as “A” provided an EC₅₀<2.5 μM, Eₘₐₓ≥1.2; compounds having an activity designated as “B” provided an EC₅₀ 2.5-10 μM, Eₘₐₓ≥1.2; compounds having an activity designated as “C” provided an EC₅₀ 10-25 μM, Eₘₐₓ≥1.2; compounds having an activity designated as “D” provided an EC₅₀>25 μM, Eₘₐₓ<1.2.

TABLE 3
Efficacy and Potency of Compounds In Increasing
the Expression of ABCB11 E297G

| Compound No. | BSEP EC₅₀ (μM) |
|---|---|
| I-1 | B |
| I-25 | B |
| I-13 | C |
| I-177 | B |
| I-176 | A |
| I-27 | B |
| I-26 | B |
| I-24 | C |
| I-23 | D |
| I-22 | B |
| I-21 | C |
| I-20 | B |
| I-19 | D |
| I-18 | D |
| I-17 | A |
| I-16 | C |
| I-15 | B |
| I-14 | D |
| I-12 | B |
| I-11 | A |
| I-10 | D |
| I-9 | D |
| I-8 | A |
| I-7 | D |
| I-6 | B |
| I-5 | B |
| I-4 | D |
| I-3 | D |
| I-2 | B |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### REFERENCES

1. Vauthier, V. et al. (2017) Targeted pharmacotherapies for defective ABC transporters. Biochem Pharmacol 136, 1-11. 10.1016/j.bcp.2017.02.020
2. Dean, M. et al. (2022) The human ATP-binding cassette (ABC) transporter superfamily. Hum Mutat 43, 1162-1182. 10.1002/humu.24418
3. Liu, X. (2019) ABC Family Transporters. Adv Exp Med Biol 1141, 13-100. 10.1007/978-981-13-7647-4_2
4. Molday, R. S. et al. (2022) Structure and function of ABCA4 and its role in the visual cycle and Stargardt macular degeneration. Prog Retin Eye Res 89, 101036. 10.1016/j.preteyeres.2021.101036
5. Kroll, T. et al. (2021) Structure and Function of Hepatobiliary ATP Binding Cassette Transporters. Chem Rev 121, 5240-5288. 10.1021/acs.chemrev.0c00659
6. Wanders, R. J. et al. (2007) The peroxisomal ABC transporter family. Pflugers Arch 453, 719-734. 10.1007/s00424-006-0142-x
7. Shaughnessy, C. A. et al. (2021) Elexacaftor is a CFTR potentiator and acts synergistically with ivacaftor during acute and chronic treatment. Sci Rep 11, 19810. 10.1038/s41598-021-99184-1
8. Thomas. C. and Tampe, R. (2020) Structural and Mechanistic Principles of ABC Transporters. Annu Rev Biochem 89, 605-636. 10.1146/annurev-biochem-011520-105201
9. Byrne. J. A. et al. (2009) Missense mutations and single nucleotide polymorphisms in ABCB11 impair bile salt export pump processing and function or disrupt pre-messenger RNA splicing. Hepatology 49, 553-567. 10.1002/hep.22683
10. Gunaydin, M. and Bozkurter Cil, A. T. (2018) Progressive familial intrahepatic cholestasis: diagnosis, management, and treatment. Hepat Med 10, 95-104. 10.2147/HMER.S137209
11. Bosma, P. J. et al. (2020) Gene Therapy for Progressive Familial Intrahepatic Cholestasis: Current Progress and Future Prospects. Int J Mol Sci 22. 10.3390/ijms22010273
12. Boyer, J. L. (2013) Bile formation and secretion. Compr Physiol 3, 1035-1078. 10.1002/cphy.c120027
13. Gerloff, T. et al. (1998) The sister of P-glycoprotein represents the canalicular bile salt export pump of mammalian liver. J Biol Chem 273, 10046-10050. 10.1074/jbc.273.16.10046
14. van der Woerd, W. L. et al. (2017) Current and future therapies for inherited cholestatic liver diseases. World J Gastroenterol 23, 763-775. 10.3748/wjg.v23.i5.763
15. Wang, L. et al. (2020) Cryo-EM structure of human bile salts exporter ABCB11. Cell Res 30, 623-625. 10.1038/s41422-020-0302-0
16. Boyer, J. L. and Soroka, C. J. (2021) Bile formation and secretion: An update. J Hepatol 75, 190-201. 10.1016/j.jhep.2021.02.011
17. Al-Dury. S. and Marschall, H. U. (2018) lleal Bile Acid Transporter Inhibition for the Treatment of Chronic Constipation, Cholestatic Pruritus, and NASH. Front Pharmacol 9, 931. 10.3389/fphar.2018.00931
18. van Wessel, D. B. E. et al. (2020) Genotype correlates with the natural history of severe bile salt export pump deficiency. J Hepatol 73, 84-93. 10.1016/j.jhep.2020.02.007
19. Stattermayer, A. F. et al. (2020) Variants in ABCB4 (MDR3) across the spectrum of cholestatic liver diseases in adults. J Hepatol 73, 651-663. 10.1016/j.jhep.2020.04.036
20. Nayagam, J. S. et al. (2022) Clinical phenotype of adult-onset liver disease in patients with variants in ABCB4, ABCB11, and ATP8B1. Hepatol Commun. 10.1002/hep4.2051
21. Stapelbroek, J. M. et al. (2010) Liver disease associated with canalicular transport defects: current and future therapies. J Hepatol 52, 258-271. 10.1016/j.jhep.2009.11.012
22. Thompson, R. J. et al. (2022) Odevixibat treatment in progressive familial intrahepatic cholestasis: a randomised, placebo-controlled, phase 3 trial. Lancet Gastroenterol Hepatol 7, 830-842. 10.1016/S2468-1253(22)00093-0
23. Le Saux, O. et al. (2000) Mutations in a gene encoding an ABC transporter cause pseudoxanthoma elasticum. Nat Genet 25, 223-227. 10.1038/76102
24. Iwanaga, A. and Murota, H. (2022) Author reply to comment on “Clinical practice guidelines for pseudoxanthoma elasticum (2017)”: The importance of mutation analysis. J Dermatol 49, e250. 10.1111/1346-8138.16379
25. Borst, P. et al. (2019) PXE, a Mysterious Inborn Error Clarified. Trends Biochem Sci 44, 125-140. 10.1016/j.tibs.2018.10.005
26. Nitschke, Y. and Rutsch, F. (2012) Generalized arterial calcification of infancy and pseudoxanthoma elasticum: two sides of the same coin. Front Genet 3, 302. 10.3389/fgene.2012.00302
27. Ferreira. C. R. et al. (2021) Ectopic Calcification and Hypophosphatemic Rickets: Natural History of ENPP1 and ABCC6 Deficiencies. J Bone Miner Res 36, 2193-2202. 10.1002/jbmr.4418
28. Bartstra, J. W. et al. (2021) Genotype-phenotype correlation in pseudoxanthoma elasticum. Atherosclerosis 324, 18-26. 10.1016/j.atherosclerosis.2021.03.012
29. Matsuzaki, Y. et al. (2005) Tissue-specific expression of the ABCC6 gene. J Invest Dermatol 125, 900-905. 10.1111/j.0022-202X.2005.23897.x
30. Scheffer. G. L. et al. (2002) MRP6 (ABCC6) detection in normal human tissues and tumors. Lab Invest 82, 515-518. 10.1038/labinvest.3780444
31. Jansen, R. S. et al. (2014) ABCC6-mediated ATP secretion by the liver is the main source of the mineralization inhibitor inorganic pyrophosphate in the systemic circulation-brief report. Arterioscler Thromb Vasc Biol 34, 1985-1989. 10.1161/ATVBAHA.114.304017
32. Jansen, R. S. et al. (2013) ABCC6 prevents ectopic mineralization seen in pseudoxanthoma elasticum by inducing cellular nucleotide release. Proc Natl Acad Sci USA 110, 20206-20211. 10.1073/pnas.1319582110
33. Iwanaga. A. et al. (2022) Clinical practice guidelines for pseudoxanthoma elasticum (2017): Clinical Practice Guidelines for Pseudoxanthoma Elasticum Drafting Committee. J Dermatol 49, e91-e98. 10.1111/1346-8138.16301
34. Fong, V. and Patel, S. B. (2021) Recent advances in ABCG5 and ABCG8 variants. Curr Opin Lipidol 32, 117-122. 10.1097/MOL.0000000000000734
35. Hooper, A. J. et al. (2017) Clinical utility gene card for: Sitosterolaemia. Eur J Hum Genet 25. 10.1038/ejhg.2016.187
36. Bhattacharyya, A. K. and Connor, W. E. (1974) Beta-sitosterolemia and xanthomatosis. A newly described lipid storage disease in two sisters. J Clin Invest 53, 1033-1043. 10.1172/JCI107640
37. Patel, S. B. et al. (1998) Sitosterolemia: exclusion of genes involved in reduced cholesterol biosynthesis. J Lipid Res 39, 1055-1061
38. Williams, K. et al. (2021) Sitosterolemia: Twenty Years of Discovery of the Function of ABCG5ABCG8. Int J Mol Sci 22. 10.3390/ijms22052641
39. Graf. G. A. et al. (2002) Coexpression of ATP-binding cassette proteins ABCG5 and ABCG8 permits their transport to the apical surface. J Clin Invest 110, 659-669. 10.1172/JCI16000
40. Jakulj, L. et al. (2016) Transintestinal Cholesterol Transport Is Active in Mice and Humans and Controls Ezetimibe-Induced Fecal Neutral Sterol Excretion. Cell Metab 24, 783-794. 10.1016/j.cmet.2016.10.001
41. Zein, A. A. et al. (2019) ABCG5/G8: a structural view to pathophysiology of the hepatobiliary cholesterol secretion. Biochem Soc Trans 47, 1259-1268. 10.1042/BST20190130
42. Chen, Z. C. et al. (2008) Significant association of ABCG8:D19H gene polymorphism with hypercholesterolemia and insulin resistance. J Hum Genet 53, 757-763. 10.1007/s10038-008-0310-2
43. Rust, S. et al. (1999) Tangier disease is caused by mutations in the gene encoding ATP-binding cassette transporter 1. Nat Genet 22, 352-355. 10.1038/11921
44. Schaefer, E. J. et al. (2016) Diagnosis and treatment of high density lipoprotein deficiency. Prog Cardiovasc Dis 59, 97-106. 10.1016/j.pcad.2016.08.006
45. Maranghi, M. et al. (2019) A novel splicing mutation in the ABCA1 gene, causing Tangier disease and familial HDL deficiency in a large family. Biochem Biophys Res Commun 508, 487-493. 10.1016/j.bbrc.2018.11.064
46. Koseki, M. et al. (2021) Current Diagnosis and Management of Tangier Disease. J Atheroscler Thromb 28, 802-810. 10.5551/jat.RV17053
47. Langmann, T. et al. (1999) Molecular cloning of the human ATP-binding cassette transporter 1 (hABC1): evidence for sterol-dependent regulation in macrophages. Biochem Biophys Res Commun 257, 29-33. 10.1006/bbrc.1999.0406
48. Hoekstra, M. et al. (2003) Specific gene expression of ATP-binding cassette transporters and nuclear hormone receptors in rat liver parenchymal, endothelial, and Kupffer cells. J Biol Chem 278, 25448-25453. 10.1074/jbc.M301189200
49. Wang, X. et al. (2007) Macrophage ABCA1 and ABCG1, but not SR—BI, promote macrophage reverse cholesterol transport in vivo. J Clin Invest 117, 2216-2224. 10.1172/JCI32057
50. Oram, J. F. and Lawn, R. M. (2001) ABCA1. The gatekeeper for eliminating excess tissue cholesterol. J Lipid Res 42, 1173-1179
51. Oram, J. F. et al. (2000) ABCA1 is the cAMP-inducible apolipoprotein receptor that mediates cholesterol secretion from macrophages. J Biol Chem 275, 34508-34511. 10.1074/jbc.M006738200
52. Brunham, L. R. et al. (2007) Beta-cell ABCA1 influences insulin secretion, glucose homeostasis and response to thiazolidinedione treatment. Nat Med 13, 340-347. 10.1038/nm1546
53. Wang. N. et al. (2000) Specific binding of ApoA-L, enhanced cholesterol efflux, and altered plasma membrane morphology in cells expressing ABC1. J Biol Chem 275, 33053-33058. 10.1074/jbc.M005438200
54. Jacobo-Albavera, L. et al. (2021) The Role of the ATP-Binding Cassette A1 (ABCA1) in Human Disease. Int J Mol Sci 22. 10.3390/ijms22041593
55. Al-Khuzaei, S. et al. (2021) An Overview of the Genetics of ABCA4 Retinopathies, an Evolving Story. Genes (Basel) 12. 10.3390/genes12081241
56. Tsang, S. and Sharma, T. (2018) Stargardt disease. In Atlas of Inherited Retinal Diseases (Tsang, S. and Sharma, T., eds). pp. 140-151, Springer International Publishing AG
57. Tanna, P. et al. (2017) Stargardt disease: clinical features, molecular genetics, animal models and therapeutic options. Br J Ophthalmol 101, 25-30. 10.1136/bjophthalmol-2016-308823
58. Kawaguchi, K. and Morita, M. (2016) ABC Transporter Subfamily D: Distinct Differences in Behavior between ABCD1-3 and ABCD4 in Subcellular Localization, Function, and Human Disease. Biomed Res Int 2016, 6786245. 10.1155/2016/6786245
59. Turk, B. R. et al. (2020) X-linked adrenoleukodystrophy: Pathology, pathophysiology, diagnostic testing, newborn screening and therapies. Int J Dev Neurosci 80, 52-72. 10.1002/jdn.10003
60. Barthel, A. et al. (2019) An Update on Addison's Disease. Exp Clin Endocrinol Diabetes 127, 165-175. 10.1055/a-0804-2715
61. Kawaguchi, K. et al. (2016) Translocation of the ABC transporter ABCD4 from the endoplasmic reticulum to lysosomes requires the escort protein LMBD1. Sci Rep 6, 30183. 10.1038/srep30183
62. Weinhofer. I. et al. (2018) Impaired plasticity of macrophages in X-linked adrenoleukodystrophy. Brain 141, 2329-2342. 10.1093/brain/awv127
63. Beers, M. F. and Mulugeta, S. (2017) The biology of the ABCA3 lipid transporter in lung health and disease. Cell Tissue Res 367, 481-493. 10.1007/s00441-016-2554-z
64. Flamein, F. et al. (2012) Molecular and cellular characteristics of ABCA3 mutations associated with diffuse parenchymal lung diseases in children. Hum Mol Genet 21, 765-775. 10.1093/hmg/ddr508
65. Hammel, M. et al. (2007) Targeted inactivation of the murine Abca3 gene leads to respiratory failure in newborns with defective lamellar bodies. Biochem Biophys Res Commun 359, 947-951. 10.1016/j.bbrc.2007.05.219
66. Wambach, J. A. et al. (2022) First Steps toward Personalized Therapies for ABCA3 Deficiency. Am J Respir Cell Mol Biol 66, 349-350. 10.1165/rcmb.2021-0405ED
67. Sanders, M. et al. (2021) Genomic, transcriptomic, and protein landscape profile of CFTR and cystic fibrosis. Hum Genet 140, 423-439. 10.1007/s00439-020-02211-w
68. Amaral, M. D. (2015) Novel personalized therapies for cystic fibrosis: treating the basic defect in all patients. J Intern Med 277, 155-166. 10.1111/joim.12314
69. Breijyeh, Z. and Karaman, R. (2020) Comprehensive Review on Alzheimer's Disease: Causes and Treatment. Molecules 25. 10.3390/molecules25245789
70. Anstey, K. J. et al. (2017) Updating the Evidence on the Association between Serum Cholesterol and Risk of Late-Life Dementia: Review and Meta-Analysis. J Alzheimers Dis 56, 215-228. 10.3233/JAD-160826
71. Torrandell-Haro, G. et al. (2020) Statin therapy and risk of Alzheimer's and age-related neurodegenerative diseases. Alzheimers Dement (NY) 6, e12108. 10.1002/trc2.12108
72. Bossaerts, L. et al. (2022) The role of ATP-binding cassette subfamily A in the etiology of Alzheimer's disease. Mol Neurodegener 17, 31. 10.1186/si3024-022-00536-w
73. Koldamova, R. P. et al. (2005) The liver X receptor ligand T0901317 decreases amyloid beta production in vitro and in a mouse model of Alzheimer's disease. J Biol Chem 280, 4079-4088. 10.1074/jbc.M411420200
74. Koldamova, R. et al. (2005) Lack of ABCA1 considerably decreases brain ApoE level and increases amyloid deposition in APP23 mice. J Biol Chem 280, 43224-43235. 10.1074/jbc.M504513200
75. Zhu. X. et al. (2010) Macrophage ABCA1 reduces MyD88-dependent Toll-like receptor trafficking to lipid rafts by reduction of lipid raft cholesterol. J Lipid Res 51, 3196-3206. 10.1194/jlr.M006486
76. Sviridov, D. et al. (2020) Lipid rafts as a therapeutic target. J Lipid Res 61, 687-695. 10.1194/jlr.TR120000658
77. Ehehalt, R. et al. (2003) Amyloidogenic processing of the Alzheimer beta-amyloid precursor protein depends on lipid rafts. J Cell Biol 160, 113-123. 10.1083/jcb.200207113
78. Chov, R. W. et al. (2012) Amyloid precursor protein (APP) traffics from the cell surface via endosomes for amyloid beta (Abeta) production in the trans-Golgi network. Proc Natl Acad Sci USA 109, E2077-2082. 10.1073/pnas.1208635109
79. Marquer, C. et al. (2011) Local cholesterol increase triggers amyloid precursor protein-Bacel clustering in lipid rafts and rapid endocytosis. FASEB J 25, 1295-1305. 10.1096/fj.10-168633
80. Wahrle, S. E. et al. (2005) Deletion of Abca1 increases Abeta deposition in the PDAPP transgenic mouse model of Alzheimer disease. J Biol Chem 280. 43236-43242. 10.1074/jbc.M508780200
81. Lefterov. I. et al. (2009) Memory deficits in APP23/Abca1+/− mice correlate with the level of Abeta oligomers. ASN Neuro 1. 10.1042/AN20090015
82. Jiang, Q. et al. (2008) ApoE promotes the proteolytic degradation of Abeta. Neuron 58, 681-693. 10.1016/j.neuron.2008.04.010
83. Leissring, M. A. et al. (2003) Enhanced proteolysis of beta-amyloid in APP transgenic mice prevents plaque formation, secondary pathology, and premature death. Neuron 40, 1087-1093. 10.1016/s0896-6273(03)00787-6
84. Husain, M. A. et al. (2021) APOE and Alzheimer's Disease: From Lipid Transport to Physiopathology and Therapeutics. Front Neurosci 15, 630502. 10.3389/fnins.2021.630502
85. Wahrle, S. E. et al. (2008) Overexpression of ABCA1 reduces amyloid deposition in the PDAPP mouse model of Alzheimer disease. J Clin Invest 118, 671-682. 10.1172/JCI33622
86. Boehm-Cagan, A. et al. (2016) ABCA1 Agonist Reverses the ApoE4-Driven Cognitive and Brain Pathologies. J Alzheimers Dis 54, 1219-1233. 10.3233/JAD-160467
87. Noveir, S. D. et al. (2022) Effect of the ABCA1 agonist CS-6253 on amyloid-beta and lipoprotein metabolism in cynomolgus monkeys. Alzheimers Res Ther 14, 87. 10.1186/s13195-022-01028-1
88. Reitz, C. et al. (2013) Variants in the ATP-binding cassette transporter (ABCA7), apolipoprotein E 4, and the risk of late-onset Alzheimer disease in African Americans. JAMA 309, 1483-1492. 10.1001/jama.2013.2973
89. Cukier, H. N. et al. (2016) ABCA7 frameshift deletion associated with Alzheimer disease in African Americans. Neurol Genet 2, e79. 10.1212/NXG.0000000000000079
90. Bellenguez, C. et al. (2017) Contribution to Alzheimer's disease risk of rare variants in TREM2, SORL1, and ABCA7 in 1779 cases and 1273 controls. Neurobiol Aging 59, 220 e221-220 e229. 10.1016/j.neurobiolaging.2017.07.001
91. De Roeck, A. et al. (2017) Deleterious ABCA7 mutations and transcript rescue mechanisms in early onset Alzheimer's disease. Acta Neuropathol 134, 475-487. 10.1007/s00401-017-1714-x
92. Apostolova, L. G. et al. (2018) Associations of the Top 20 Alzheimer Disease Risk Variants With Brain Amyloidosis. JAMA Neurol 75, 328-341. 10.1001/jamaneurol.2017.4198
93. Stage, E. et al. (2016) The effect of the top 20 Alzheimer disease risk genes on gray-matter density and FDG PET brain metabolism. Alzheimers Dement (Amst) 5, 53-66. 10.1016/j.dadm.2016.12.003
94. Lyssenko, N. N. and Pratico, D. (2021) ABCA7 and the altered lipidostasis hypothesis of Alzheimer's disease. Alzheimers Dement 17, 164-174. 10.1002/alz.12220
95. Lamartiniere, Y. et al. (2018) ABCA7 Downregulation Modifies Cellular Cholesterol Homeostasis and Decreases Amyloid-beta Peptide Efflux in an in vitro Model of the Blood-Brain Barrier. J Alzheimers Dis 64, 1195-1211. 10.3233/JAD-170883
96. Tanaka, N. et al. (2010) Helical apolipoproteins of high-density lipoprotein enhance phagocytosis by stabilizing ATP-binding cassette transporter A7. J Lipid Res 51, 2591-2599. 10.1194/jlr.M006049
97. Jehle, A. W. et al. (2006) ATP-binding cassette transporter A7 enhances phagocytosis of apoptotic cells and associated ERK signaling in macrophages. J Cell Biol 174, 547-556. 10.1083/jcb.200601030
98. Aikawa, T. et al. (2019) ABCA7 haplodeficiency disturbs microglial immune responses in the mouse brain. Proc Natl Acad Sci USA 116, 23790-23796. 10.1073/pnas.1908529116
99. Satoh, K. et al. (2015) ATP-binding cassette transporter A7 (ABCA7) loss of function alters Alzheimer amyloid processing. J Biol Chem 290, 24152-24165. 10.1074/jbc.M115.655076
100. Sakae, N. et al. (2016) ABCA7 Deficiency Accelerates Amyloid-beta Generation and Alzheimer's Neuronal Pathology. J Neurosci 36, 3848-3859. 10.1523/JNEUROSCI.3757-15.2016
101. Kim. W. S. et al. (2013) Deletion of Abca7 increases cerebral amyloid-beta accumulation in the J20 mouse model of Alzheimer's disease. J Neurosci 33, 4387-4394. 10.1523/JNEUROSCI.4165-12.2013

1. A compound represented by Formula I: 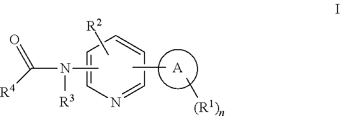
or a pharmaceutically acceptable salt thereof; wherein:
Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur, a 4-8 membered saturated or partially unsaturated monocyclic heterocyclic ring containing 1-4 heteroatoms independently selected from nitrogen, oxygen, and sulfur, a 5-6 membered monocyclic heteroaromatic ring having 1-4 heteroatoms independently selected from nitrogen, oxygen, and sulfur, a 6-11 membered saturated or partially unsaturated bicyclic carbocyclic ring, a 3-8 membered saturated or partially unsaturated monocyclic carbocyclic ring, an 8-10 membered bicyclic aromatic carbocyclic ring, or phenyl;
R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, C₁₋₄ haloaliphatic, or a cyclic group selected from a 3-8 membered saturated monocyclic carbocyclic ring or a 3-8 membered saturated monocyclic heterocyclic ring having 1-4 heteroatoms independently selected from nitrogen, oxygen, and sulfur;
R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy;
R³ is hydrogen, cyclopropyl, or C₁₋₃ alkyl optionally substituted with a group selected from —OH, OMe, —NH₂, —NH(CH₃), and —N(CH₃)₂;
R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵;
R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur;
R represents independently for each occurrence hydrogen, hydroxyl, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring;
n is 0, 1, 2, or 3; and
p is 0, 1, 2, 3 or 4.

2. The compound of claim 1, wherein the compound is a compound of Formula I.

3. The compound of either of claim 1 or 2, wherein Ring A is a 6-14 membered saturated or partially unsaturated bicyclic heterocyclic ring containing 1-4 heteroatoms independently selected from nitrogen, oxygen, and sulfur; an 8-10 membered bicyclic heteroaromatic ring having 1-4 heteroatoms independently selected from nitrogen, oxygen, and sulfur, a 4-8 membered saturated or partially unsaturated monocyclic heterocyclic ring containing 1-4 heteroatoms independently selected from nitrogen, oxygen, and sulfur, a 5-6 membered monocyclic heteroaromatic ring having 1-4 heteroatoms independently selected from nitrogen, oxygen, and sulfur, an 8-10 membered bicyclic aromatic carbocyclic ring, or phenyl.

4. The compound of any one of claims 1-3, wherein Ring A is a 6-14 membered saturated or partially unsaturated bicyclic heterocyclic ring containing 1-4 heteroatoms independently selected from nitrogen, oxygen, and sulfur or an 8-10 membered bicyclic heteroaromatic ring having 1-4 heteroatoms independently selected from nitrogen, oxygen, and sulfur.

5. The compound of any one of claims 1-3, wherein Ring A is a 6-11 membered saturated or partially unsaturated bicyclic carbocyclic ring, or a 3-8 membered saturated or partially unsaturated monocyclic carbocyclic ring, or phenyl.

6. The compound of any one of claims 1-5, wherein R¹ is —H, —CH₃, —CH₂C(O)NH₂, —CH₂OCH₃, —CH₂CH₂OCH₃, —NHAc, —OH, —C(O)CH₂OH, —C(O)CH₃, —CH₂C(O)NH₂, —OCH₃, CH₂OCH₃, —C(O)OCH₃, —C(O)OH, —NHCH₂CH₂OH, —CH₂CH₂C(O)OCH₃, —NHS(O)₂CH₃, —C(O)NH₂, —S(O)₂CH₃, Bn, —OC(CH₃)₂, —NHCH₂CH₂OCH₃, —F, —Cl, —Br, (O), —CH₂NH₂, —NH₂, —NHC(O)CH₃, —C(O)OCH₂CH₃, —O-cyclopentyl, —S(O)₂NHCH₂CH₂OH, —CH₂CH₃, —N(CH₃)₂, —CH(CH₃)₂, —CF₃, —CN, —S(O)₂CH₃, —CH₂OH, —OCH₃, —CH₂N(CH₃)₂, —CH₂NHCH₂CH₂OH, —O(CH₂)₃N(CH₃)₂, —C(O)NH₂, cyclohexyl, cyclopropyl, —OCF, —S(O)₂NH₂, —CHF₂, —CH₂NHC(O)OC(CH₃)₃, —C(CH₃)₂(OH), —CH(OH)CH₃, or —O(CH₂)₂NR₂.

7. The compound of any one of claims 1-6, wherein R² is hydrogen.

8. The compound of any one of claims 1-6, wherein R² is C₁₋₃ alkyl.

9. The compound of any one of claims 1-6, wherein R² is methyl.

10. The compound of any one of claims 1-6, wherein R² is cyclopropyl.

11. The compound of any one of claims 1-10, wherein R³ is hydrogen.

12. The compound of any one of claims 1-10, wherein R³ is C₁₋₃ alkyl.

13. The compound of any one of claims 1-10, wherein R³ is cyclopropyl.

14. The compound of any one of claims 1-10, wherein R³ is C₁₋₃ alkyl substituted with a group selected from —OH, —OMe, —NH₂, —NH(CH₃), and —N(CH₃)₂.

15. The compound of claim 14, wherein the substitution on C₁₋₃ alkyl occurs on the C₁₋₃ alkyl terminal carbon atom.

16. The compound of any one of claims 1-6, wherein R² is methyl and R³ is hydrogen.

17. The compound of any one of claims 1-3 or 6-16, wherein the compound is of Formula I-A, I-B, or I-C: 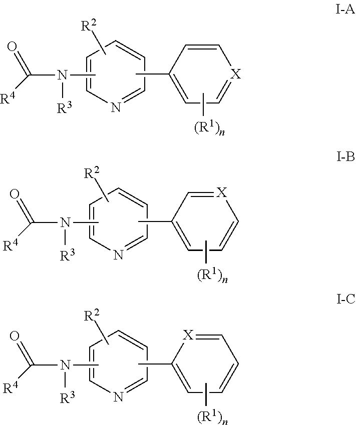
or a pharmaceutically acceptable salt thereof, wherein:
X is nitrogen or carbon.

18. The compound of claim 17, wherein X is nitrogen.

19. The compound of any one of claims 1-4 or 6-16, wherein the compound is of Formula I-D: 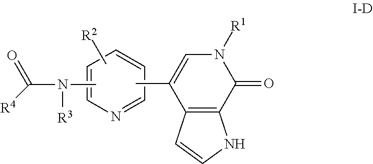
or a pharmaceutically acceptable salt thereof.

20. The compound of claim 19, wherein R¹ is —CH₃, R² is —CH₃, and R³ is —H.

21. A compound represented by Formula II: 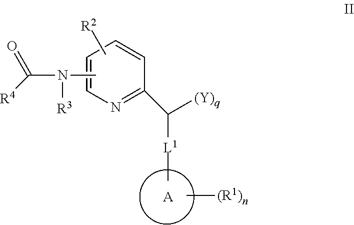
or a pharmaceutically acceptable salt thereof; wherein:
Ring A is a 6-14 membered saturated or partially unsaturated bicyclic heterocyclic ring containing 1-4 heteroatoms independently selected from nitrogen, oxygen, and sulfur, an 8-10 membered bicyclic heteroaromatic ring having 1-4 heteroatoms independently selected from nitrogen, oxygen, and sulfur, a 4-8 membered saturated or partially unsaturated monocyclic heterocyclic ring containing 1-4 heteroatoms independently selected from nitrogen, oxygen, and sulfur, a 5-6 membered monocyclic heteroaromatic ring having 1-4 heteroatoms independently selected from nitrogen, oxygen, and sulfur, a 6-11 membered saturated or partially unsaturated bicyclic carbocyclic ring, a 3-8 membered saturated or partially unsaturated monocyclic carbocyclic ring, an 8-10 membered bicyclic aromatic carbocyclic ring, or phenyl;
R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, or C₁₋₄ haloaliphatic, or a cyclic group selected from a 3-8 membered saturated monocyclic carbocyclic ring or a 3-8 membered saturated monocyclic heterocyclic ring having 1-3 heteroatoms independently selected from nitrogen, oxygen, and sulfur;
R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy;
R³ is hydrogen, cyclopropyl, or C₁₋₃ alkyl optionally substituted with a group selected from —OH, —OMe, —NH₂, —NH(CH₃), and —N(CH₃)₂;
R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-4 heteroatoms independently selected from nitrogen, oxygen, and sulfur, wherein R⁴ is substituted with p instances of R⁵;
R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur;
L¹ is a bond, —OCH₂—, —O—, —OCH₂CH₂—, —NHCH₂—, —NHCH₂CH₂—, or —NH—;
Y represents independently for each occurrence —R, oxo, —OR, —NR₂, —OCH₂CHF₂, —OCH₂CH₂OH, or —NHCH₂CH₂OR; or two Y groups attached to the same carbon atom can together form a 3-6 membered saturated or partially unsaturated monocyclic carbocyclic ring or a 3-8 membered saturated or partially unsaturated monocyclic heterocyclic ring having 1-4 heteroatoms independently selected from nitrogen, oxygen, and sulfur;
R represents independently for each occurrence —H, —OH, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring;
n is 0, 1, 2, or 3;
p is 0, 1, 2, 3 or 4; and
q is 0, 1 or 2.

22. The compound of claim 21, wherein the compound is a compound of Formula II.

23. The compound of either of claim 21 or claim 22, wherein Ring A is a 6-14 membered saturated or partially unsaturated bicyclic heterocyclic ring containing 1-4 heteroatoms independently selected from nitrogen, oxygen, and sulfur, an 8-10 membered bicyclic heteroaromatic ring having 1-4 heteroatoms independently selected from nitrogen, oxygen, and sulfur, a 4-8 membered saturated or partially unsaturated monocyclic heterocyclic ring containing 1-4 heteroatoms independently selected from nitrogen, oxygen, and sulfur, or a 5-6 membered monocyclic heteroaromatic ring having 1-4 heteroatoms independently selected from nitrogen, oxygen, and sulfur.

24. The compound of any one of claims 21-23, wherein Ring A is a 6-14 membered saturated or partially unsaturated bicyclic heterocyclic ring containing 1-4 heteroatoms independently selected from nitrogen, oxygen, and sulfur or an 8-10 membered bicyclic heteroaromatic ring having 1-4 heteroatoms independently selected from nitrogen, oxygen, and sulfur.

25. The compound of any one of claims 21-23, wherein Ring A is a 6-11 membered saturated or partially unsaturated bicyclic carbocyclic ring, a 3-8 membered saturated or partially unsaturated monocyclic carbocyclic ring, an 8-10 membered bicyclic aromatic carbocyclic ring, or phenyl.

26. The compound of any one of claims 21-25, wherein R¹ is —H, —CH₃, —CH₂C(O)NH₂, —CH₂OCH₃, —CH₂CH₂OCH₃, —NHAc, —OH, —C(O)CH₂OH, —C(O)CH₃, —CH₂C(O)NH₂, —OCH₃, CH₂OCH₃, —C(O)OCH₃, —C(O)OH, —NHCH₂CH₂OH, —CH₂CH₂C(O)OCH₃, —NHS(O)₂CH₃, —C(O)NH₂, —S(O)₂CH₃, Bn, —OC(CH₃)₂, —NHCH₂CH₂OCH₃, —F, —Cl, —Br, (O), —CH₂NH₂, —NH₂, —NHC(O)CH₃, —C(O)OCH₂CH₃, —O-cyclopentyl, —S(O)₂NHCH₂CH₂OH, —CH₂CH₃, —N(CH₃)₂, —CH(CH₃)₂, —CF₃, —CN, —S(O)₂CH₃, —CH₂OH, —OCH₃, —CH₂N(CH₃)₂, —CH₂NHCH₂CH₂OH —O(CH₂)₃N(CH₃)₂, —C(O)NH₂, cyclohexyl, cyclopropyl, —OCF₃, —S(O)₂NH₂, —CHF₂, —CH₂NHC(O)OC(CH₃)₃, —C(CH₃)₂(OH), —CH(OH)CH₃, or —O(CH₂)₂NR₂.

27. The compound of any one of claims 21-26, wherein R² is hydrogen.

28. The compound of any one of claims 21-26, wherein R² is C₁₋₃ alkyl.

29. The compound of any one of claims 21-26, wherein R² is methyl.

30. The compound of any one of claims 21-26, wherein R² is cyclopropyl.

31. The compound of any one of claims 21-30, wherein R³ is hydrogen.

32. The compound of any one of claims 21-30, wherein R³ is C₁₋₃ alkyl.

33. The compound of any one of claims 21-30, wherein R³ is cyclopropyl.

34. The compound of any one of claims 21-30, wherein R³ is C₁₋₃ alkyl substituted with a group selected from —OH, —OMe, —NH₂, —NH(CH₃), and —N(CH₃)₂.

35. The compound of claim 34, wherein the substitution on C₁₋₃ alkyl occurs on the C₁₋₃ alkyl terminal carbon atom.

36. The compound of any one of claims 21-26, wherein R² is methyl and R³ is hydrogen.

37. The compound of any one of claims 21-36, wherein L¹ is a bond, —O—, or —NH—.

38. The compound of claim 37, wherein L¹ is a bond.

39. A compound represented by Formula III: 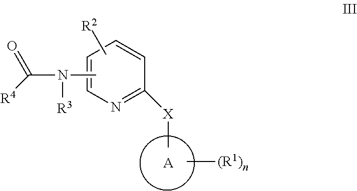
or a pharmaceutically acceptable salt thereof; wherein:
Ring A is a 6-14 membered saturated or partially unsaturated bicyclic heterocyclic ring containing 1-4 heteroatoms independently selected from nitrogen, oxygen, and sulfur, an 8-10 membered bicyclic heteroaromatic ring having 1-4 heteroatoms independently selected from nitrogen, oxygen, and sulfur, a 4-8 membered saturated or partially unsaturated monocyclic heterocyclic ring containing 1-4 heteroatoms independently selected from nitrogen, oxygen, and sulfur, a 5-6 membered monocyclic heteroaromatic ring having 1-4 heteroatoms independently selected from nitrogen, oxygen, and sulfur, a 6-11 membered saturated or partially unsaturated bicyclic carbocyclic ring, a 3-8 membered saturated or partially unsaturated monocyclic carbocyclic ring, an 8-10 membered bicyclic aromatic carbocyclic ring, or phenyl;
R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, or C₁₋₄ haloaliphatic, or a cyclic group selected from a 3-8 membered saturated monocyclic carbocyclic ring or a 3-8 membered saturated monocyclic heterocyclic ring having 1-3 heteroatoms independently selected from nitrogen, oxygen, and sulfur;
R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy;
R³ is hydrogen, cyclopropyl, or C₁₋₃ alkyl optionally substituted with a group selected from —OH, —OMe, —NH₂, —NH(CH₃), and —N(CH₃)₂;
R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵;
R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur;
X is —O— or —NH—;
R represents independently for each occurrence —H, —OH, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, and a 3-7 membered saturated or partially unsaturated monocyclic carbocyclic ring;
n is 0, 1, 2, or 3; and
p is 0, 1, 2, 3 or 4.

40. The compound of claim 39, wherein the compound is a compound of Formula III.

41. The compound of either of claim 39 or claim 40, wherein Ring A is a 6-14 membered saturated or partially unsaturated bicyclic heterocyclic ring containing 1-4 heteroatoms independently selected from nitrogen, oxygen, and sulfur, an 8-10 membered bicyclic heteroaromatic ring having 1-4 heteroatoms independently selected from nitrogen, oxygen, and sulfur, a 4-8 membered saturated or partially unsaturated monocyclic heterocyclic ring containing 1-4 heteroatoms independently selected from nitrogen, oxygen, and sulfur, or a 5-6 membered monocyclic heteroaromatic ring having 1-4 heteroatoms independently selected from nitrogen, oxygen, and sulfur.

42. The compound of any one of claims 39-41, wherein Ring A is a 6-14 membered saturated or partially unsaturated bicyclic heterocyclic ring containing 1-4 heteroatoms independently selected from nitrogen, oxygen, and sulfur or an 8-10 membered bicyclic heteroaromatic ring having 1-4 heteroatoms independently selected from nitrogen, oxygen, and sulfur.

43. The compound of any one of claims 39-41, wherein Ring A is a 6-11 membered saturated or partially unsaturated bicyclic carbocyclic ring, a 3-8 membered saturated or partially unsaturated monocyclic carbocyclic ring, an 8-10 membered bicyclic aromatic carbocyclic ring, or phenyl.

44. The compound of any one of claims 39-43, wherein R¹ is —H, —CH₃, —CH₂C(O)NH₂, —CH₂OCH₃, —CH₂CH₂OCH₃, —NHAc, —OH, —C(O)CH₂OH, —C(O)CH₃, —CH₂C(O)NH₂, —OCH₃, CH₂OCH₃, —C(O)OCH₃, —C(O)OH, —NHCH₂CH₂OH, —CH₂CH₂C(O)OCH₃, —NHS(O)₂CH₃, —C(O)NH₂, —S(O)₂CH₃, Bn, —OC(CH₃)₂, —NHCH₂CH₂OCH₃, —F, —Cl, —Br, (O), —CH₂NH₂, —CH₂NHCH₂CH₂OH, —NH₂, —NHC(O)CH₃, —C(O)OCH₂CH₃, —O-cyclopentyl, —S(O)₂NHCH₂CH₂OH, —CH₂CH₃, —N(CH₃)₂, —CH(CH₃)₂, —CF₃, —CN, —S(O)₂CH₃, —CH₂OH, —OCH₃, —CH₂N(CH₃)₂, —O(CH₂)₃N(CH₃)₂, —C(O)NH₂, cyclohexyl, cyclopropyl, —OCF₃, —S(O)₂NH₂, —CHF₂, —CH₂NHC(O)OC(CH₃)₃, —C(CH₃)₂(OH), —CH(OH)CH₃, or —O(CH₂)₂NR₂.

45. The compound of any one of claims 39-44, wherein R² is hydrogen.

46. The compound of any one of claims 39-44, wherein R² is C₁₋₃ alkyl.

47. The compound of any one of claims 39-44, wherein R² is methyl.

48. The compound of any one of claims 39-44, wherein R² is cyclopropyl.

49. The compound of any one of claims 39-48, wherein R³ is hydrogen.

50. The compound of any one of claims 39-48, wherein R³ is C₁₋₃ alkyl.

51. The compound of any one of claims 39-48, wherein R³ is cyclopropyl.

52. The compound of any one of claims 39-48, wherein R³ is C₁₋₃ alkyl substituted with a group selected from —OH, —OMe, —NH₂, —NH(CH₃), and —N(CH₃)₂.

53. The compound of claim 52, wherein the substitution on C₁₋₃ alkyl occurs on the C₁₋₃ alkyl terminal carbon atom.

54. The compound of any one of claims 39-44, wherein R² is methyl and R³ is hydrogen.

55. The compound of any one of claims 39-54, wherein X is —O—.

56. The compound of any one of claims 39-54, wherein X is —NH—.

57. A compound represented by Formula IV: 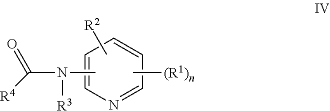
or a pharmaceutically acceptable salt thereof; wherein:
R¹ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂CH₂C(O)OR, —CH₂C(O)NR₂, —CH₂CH₂R, —CH₂CH₂OR, —CH₂NHCH₂CH₂OR, —NHCH₂CH₂OR, —NHC(O)R, —CH₂OR, —CH₂C(O)NH₂, —CH₂NHC(O)OCR₃, —OCR, C₁₋₄ aliphatic, or C₁₋₄ haloaliphatic, or a cyclic group selected from a 3-8 membered saturated monocyclic carbocyclic ring or a 3-8 membered saturated monocyclic heterocyclic ring having 1-3 heteroatoms independently selected from nitrogen, oxygen, and sulfur;
R² is hydrogen, halo, —OH, —CN, cyclopropyl, C₁₋₃ alkyl optionally substituted with a group selected from —OH, —NH₂, —NH(CH₃), and —N(CH₃)₂, C₁₋₃ haloalkyl, or C₁₋₃ alkoxy;
R³ is hydrogen, cyclopropyl, C₁₋₃ alkyl optionally substituted with a group selected from —OH, —OMe, —NH₂, —NH(CH₃), and —N(CH₃)₂;
R⁴ is a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring having 1-4 heteroatoms independently selected from nitrogen, oxygen, and sulfur, a 7-12 membered saturated or partially unsaturated bicyclic heterocyclic ring having 1-4 heteroatoms independently selected from nitrogen, oxygen, and sulfur, a 5-6 membered monocyclic heteroaromatic ring having 1-4 heteroatoms independently selected from nitrogen, oxygen, and sulfur, and an 8-10 membered bicyclic heteroaromatic ring having 1-5 heteroatoms independently selected from nitrogen, oxygen, and sulfur, wherein R⁴ is substituted with p instances of R⁵;
R⁵ represents independently for each occurrence halo, oxo, —CN, —OR, —SR, —NR₂, —S(O)₂R, —S(O)₂NR₂, —S(O)R, —S(O)NR₂, —C(O)R, —C(O)CR₃, —C(O)OR, —C(O)NR₂, —C(O)N(R)OR, —OC(O)R, —OC(O)NR₂, —O(CR₂)₃NR₂, —N(R)C(O)OR, —N(R)C(O)R, —N(R)C(O)NR₂, —N(R)C(NR)NR₂, —NR(CR₂)₂OR, —N(R)S(O)₂NR₂, —N(R)S(O)₂R, —CR₃, —CH₂NR₂, —CH₂NHCH₂CH₂OR, —CH₂CH₂C(O)OR, —CH₂C(O)NR₂, —CH₂CH₂R, —CH₂CH₂OR, —NHCH₂CH₂OR, —NHC(O)R, —CH₂OR, —CH₂C(O)NH₂, —CH₂NHC(O)OCR₃, a C₁₋₃ alkyl group, a C₁₋₃ haloalkyl group, a C₁₋₃ heteroalkyl group having 1-2 heteroatoms independently selected from nitrogen, oxygen, and sulfur, a 3-6 membered saturated or partially unsaturated monocyclic carbocyclic ring, phenyl, a 5-6 membered monocyclic heteroaromatic ring having 1-4 heteroatoms independently selected from nitrogen, oxygen, and sulfur, or a 3-6 membered saturated or partially unsaturated monocyclic heterocyclic ring having 1-4 heteroatoms independently selected from nitrogen, oxygen, and sulfur;
R represents independently for each occurrence —H, —OH, halo, or an optionally substituted group selected from C₁₋₆ aliphatic, phenyl, a 3-7 membered saturated or partially unsaturated monocyclic heterocyclic ring having 1-4 heteroatoms independently selected from nitrogen, oxygen, and sulfur, a 5-6 membered monocyclic heteroaryl ring having 1-4 heteroatoms independently selected from nitrogen, oxygen, and sulfur, a 3-7 membered saturated or partially unsaturated monocyclic carbocyclic ring;
n is 0, 1, or 2; and
p is 0, 1, 2, 3 or 4.

58. The compound of claim 57, wherein the compound is a compound of Formula IV.

59. The compound of either of claim 57 or claim 58, wherein R¹ is —H, —CH₃, —CH₂C(O)NH₂, —CH₂OCH₃, —CH₂CH₂OCH₃, —NHAc, —OH, —C(O)CH₂OH, —C(O)CH₃, —CH₂C(O)NH₂, —OCH₃, CH₂OCH₃, —C(O)OCH₃, —C(O)OH, —NHCH₂CH₂OH, —CH₂CH₂C(O)OCH₃, —NHS(O)₂CH₃, —C(O)NH₂, —S(O)₂CH₃, Bn, —OC(CH₃)₂, —NHCH₂CH₂OCH₃, —F, —Cl, —Br, (O), —CH₂NH₂, —NH₂, —NHC(O)CH₃, —C(O)OCH₂CH₃, —O— cyclopentyl, —S(O)₂NHCH₂CH₂OH, —CH₂CH₃, —N(CH₃)₂, —CH₂NHCH₂CH₂OH, —CH(CH₃)₂, —CF₃, —CN, —S(O)₂CH₃, —CH₂OH, —OCH₃, —CH₂N(CH₃)₂, —O(CH₂)₃N(CH₃)₂, —C(O)NH₂, cyclohexyl, cyclopropyl, —OCF₃, —S(O)₂NH₂, —CHF₂, —CH₂NHC(O)OC(CH₃)₃, —C(CH₃)₂(OH), —CH(OH)CH₃, or —O(CH₂)₂NR₂.

60. The compound of any one of claims 57-59, wherein R² is hydrogen.

61. The compound of any one of claims 57-59, wherein R² is C₁₋₃ alkyl.

62. The compound of any one of claims 57-59, wherein R² is methyl.

63. The compound of any one of claims 57-59, wherein R² is cyclopropyl.

64. The compound of any one of claims 57-63, wherein R³ is hydrogen.

65. The compound of any one of claims 57-63, wherein R³ is C₁₋₃ alkyl.

66. The compound of any one of claims 57-63, wherein R³ is cyclopropyl.

67. The compound of any one of claims 57-63, wherein R³ is C₁₋₃ alkyl substituted with a group selected from —OH, —OMe, —NH₂, —NH(CH₃), and —N(CH₃)₂.

68. The compound of claim 67, wherein the substitution on C₁₋₃ alkyl occurs on the C₁₋₃ alkyl terminal carbon atom.

69. The compound of any of one of claims 57-59, wherein R² is methyl and R³ is hydrogen.

70. A compound in Table 1 herein, or a pharmaceutically acceptable salt thereof.

71. A pharmaceutical composition comprising a compound of any one of claims 1-70 and a pharmaceutically acceptable carrier.

72. A method of treating a disorder associated with ABC transporter dysfunction, comprising administering to a subject in need thereof a therapeutically effective amount of a compound of any one of claims 1-70.

73. The method of claim 72, wherein the disorder associated with ABC transporter dysfunction is characterized by dysfunction in a transporter selected from one or more of ABCA1, ABCA2, ABCA3, ABCA4, ABCA5, ABCA7, ABCA12, ABCB2, ABCB3, ABCB4, ABCB6, ABCB7, ABCB10, ABCB11, ABCC1, ABCC2, ABCC4, ABCC5, ABCC6, ABCC7, ABCC8, ABCC9, ABCC12, ABCD1, ABCD2, ABCD3, ABCD4, ABCG5, ABCG8, ABCG1, and ABCG4.

74. A method of treating a disorder comprising administering to a subject in need thereof a therapeutically effective amount of a compound of any one of claims 1-70, wherein the disorder is selected from Tangier disease, Surfactant metabolism dysfunction pulmonary 3, autosomal recessive Ichthyosis congenital 4A (ARCI), Bare lymphocyte syndrome type I, Bare lymphocyte syndrome type I due to TAP2 deficiency, Dyschromatosis universalis hereditaria 3, X-linked sideroblastic anemia with ataxia, Dubin-Johnson Syndrome, Cystic fibrosis (CF), Familial Hyperinsulinemic Hypoglycemia 1, Intellectual disability Myopathy Syndrome, Congenital bile acid synthesis defect 5, Methylmalonic aciduria and homocystinuria cblJ type, Sitostrolemia, Stargardt disease, PFIC3, PFIC2, Pseudoxanthoma Elasticum, X-linked adrenoleukodystrophy (ALD), Cholestasis, Hyperbilirubinemia, Intrahepatic cholestasis of pregnancy, Biliary atresia, Alagille syndrome, primary biliary cholangitis, primary sclerosing cholangitis, NAFLD/NASH, Alzheimer's disease, Huntington's disease, Multiple sclerosis, Parkinson's disease, Hirschsprung disease, Zellweger syndrome, Type 2 diabetes, Obesity, Type 1 diabetes, Atherosclerosis, Dyslipidemia, Generalized arterial calcification of infancy, Calciphylaxis, Autosomal recessive cone-rod dystrophy, Gout, PFIC1, Myo5B deficiency cholestasis, PFIC4, and Low phospholipid associated cholelithiasis.

75. The method of claim 74, wherein the disorder is Cystic fibrosis.

76. The method of any one of claims 72-75, wherein the subject is a human.
